SEQUENCE LISTING (2 sequences)

SEQ ID NO: 1 - protein, 185 aa, Homo sapiens
  MISC_FEATURE (1)..(185): amino acid sequence of pre-pro-ADM
MKLVSVALMYLGSLAFLGADTARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 2 - protein, 48 aa, Homo sapiens
  MISC_FEATURE (1)..(48): amino acid sequence of MR-pro-ADM (amino acid residues 45-92 of pre-pro-ADM)
ELRMSSSYPTGLADVKAGPAQTLIRPQDMKGASRSPEDSSPDAARIRV

(19) United States
(12) Patent Application Publication
(10) Pub. No.: US 2018/0348235 A1
(43) Pub. Date: Dec. 6, 2018

(54) MR-proADM as marker for the extracellular volume status of a subject

(71) Applicant: B.R.A.H.M.S GmbH, Hennigsdorf (DE)

(72) Inventors: Bernard VIGUÉ, Paris (FR); Homa RAFI-NIKOUKHAH, Paris (FR)

(21) Appl. No.: 15/779,000

(22) PCT Filed: Nov. 24, 2016
(86) PCT No.: PCT/EP2016/078702

§ 371 Date: May 24, 2018

(30) Foreign Application Priority Data
Nov. 27, 2015 (EP) 15196754.4

(51) Int. Cl. G01N 33/74; G01N 33/72

(52) U.S. Cl. CPC G01N 33/74; G01N 2333/575; G01N 33/721

(57) ABSTRACT

The present invention relates to a method for determining the extracellular volume status of a subject. The method comprises determining in a sample obtained from a subject the level of the marker proadrenomedullin (proADM) or a fragment thereof, preferably MR-proADM. Further, based on the level of proADM or a fragment thereof, the fluid balance is determined and wherein said fluid balance determines the extracellular volume status. Further, based on the level of proADM or a fragment thereof, the salt balance is determined and wherein said salt balance determines the extracellular volume status and salt retention. Further, the invention relates to a method for in vitro diagnosis, risk stratification, therapy control and/or operative control of a disorder or medical condition in a subject, wherein said extracellular volume status and salt retention of said subject is determined by the herein provided method. Further, the invention relates to a kit and/or a diagnostic device for carrying out the herein provided method.

The present invention relates to the determination of the extracellular volume status of a subject, particularly of patients in health care, most particularly in intensive care. The method comprises determining in a sample obtained from a subject the level of proadrenomedullin (proADM) or fragments thereof, particularly midregional proadrenomedullin (MR-proADM). Further, based on the level of MR-proADM, the fluid balance and/or salt balance can be determined which in turn are indicative for the extracellular volume status of said subject.

### BACKGROUND OF THE INVENTION

Exact blood volumes are difficult to assess. The globular volume can be estimated by determining the hemoglobin concentration (also designated herein as “Hb”) level (Jacob, 2012). The extracellular volume can be estimated based on the weight of a subject, e.g., the body consists of 60% of water, i.e., 42 L for a 70 Kg-patient, the extracellular volume counts for 40% of body water, i.e., 17 L for a 70 Kg-patient; see FIG. 1. In clinical practice an “effective” blood volume based on the collection of dynamic information on changes in intravascular pressure and/or heart output measurements is estimated several times a day in many acute and less acute intensive care situations to guide and control prescriptions for patients. This estimation is consistently used as the basis for fundamental treatment decisions regarding volume expander quantity and catecholamine or blood transfusion use. Every day, an estimated 40% of patients in intensive care are given a volume expander following assessment (Finfer, 2011). Obtaining appropriate blood volumes while avoiding positive fluid balance is a dilemma in daily care of acute inflammatory patients, e.g., traumatic stress or sepsis. This challenge is very present in intensive care and in anesthesiology literature (Rivers et al., 2001; Chappel et al., 2008; Sakr et al., 2005; Bagshaw et al., 2008; Payen et al., 2008; Murphy et al., 2009; Boyd et al., 2011; Kelm et al., 2015; and Acheampong et al., 2015). Therapeutic recommendations have been targeted to the necessity of an acute control of higher cardiac output to guarantee an adequate oxygen delivery to organs. Fluid challenge, primarily constituted with salt and water, is the major proposed tool for volume expansion (Cecconi et al., 2009).

Increasing blood volume with salt accompanied by water causes an increase in extracellular volume, especially when diuresis is reduced by shock. Additionally, capillary permeability can be extremely increased in acute inflammatory situations, worsening the risk of accumulation of fluids (Chappell et al., 2009; Jacob et al., 2009; and Ostrowski et al., 2015). It has been recognized that the overload of volume expansion can provoke organ dysfunctions such as acute lung injury, abdominal compartment syndrome or renal dysfunction (Sakr et al., 2005; Bagshaw et al., 2008; Sakr et al., 2012; and Besen et al., 2015). Furthermore, studies also report the increase in mortality due to hydro-sodium overload (Boyd et al., 2011; Kelm et al., 2015; and Acheampong et al., 2015). A cumulative positive fluid balance of 3 to 4 kg gain of weight, or 27 to 36 g of salt resulting from a gain of 3 to 4 liters of water, is considered as the threshold where mortality and morbidity increase (Lobo et al., 2002; Brandstrup et al., 2003; and Bjerregaard et al., 2005). Therefore, the accuracy of this assessment is very important. The analytical methods used to prevent or correct these phenomena focus on “effective” blood volume based on the collection of dynamic information on changes in intravascular pressure and/or heart output measurements. Although this strategy has proven effective in the first hours of a shock, it is incapable of preventing excess plasma expansion (Hilton, 2011).

Blood transfusion prescriptions are also determined by intravascular volume. Transfusion thresholds are usually considered in light of the hemoglobin (Hb) level or the hematocrit, which is by definition the ratio of red blood cell volume to total blood volume. Numerous clinical trials carried out in various intensive care settings concede that for concentrations of between 7 and 11 g/dL of Hb, imprecision is such that it is difficult to accurately assess circulating volumes of red blood cells (Takanishi, 2008; Dorbout Mees, 2011, Jacob, 2012). Indeed, while clinical trials of broad intensive care patient populations show that transfusions are ineffective and a policy of restricting prescriptions with a threshold of 7-8 g/dL is beneficial, others performed on targeted populations show that low Hb is not favourable for prognosis (Naidech, 2007, Kellert, 2011). Moreover, these arbitrary thresholds are disputed as not accurately enough, with no respect of a clinical individual situation (Klein, 2015).

It is possible to take a direct and precise measurement of total blood volume and deduce corpuscular and plasma volume from it. However, this examination is performed rarely because it is costly, time consuming and work intensive. Therefore, this method is merely applied for specific diseases (e.g. polycythemia vera). The most reliable measurement is performed by labeling the patient's red blood cells with chromium-51 (Gore, 2005). Although this examination is accepted as the gold standard for measuring intravascular volumes, it is impossible to repeat it every day (Gore, 2005). By labeling albumin with iodine-125 to measure albumin distribution volume, it is possible to determine albumin distribution within the body. This protein is much more sensitive than red blood cells to capillary permeability impairment. As observed for the chromium-51 method, the iodine-125 method cannot be repeated every day and thus it is only applied for specific diseases. Furthermore, this kind of measurement is not suitable when instant information on the volume status of a patient is required, such as in case of intensive care unit patients.

In many intensive care units, nurses systematically measure fluid balance by daily weight or daily calculation of input and output liquids. However, in every day practice, nurses cannot devote the time necessary to collect the required information. In addition, this method is not precise and, moreover, salt balance assessment, a parameter indicating changes in extracellular volume, is never taken into account.

In health care, particularly intensive care, there is a fundamental need to improve the method of assessing the extracellular volume status of a subject in order to improve monitoring of oxygen supply to tissue and to balance the treatments in a less approximate manner Moreover, an improved method of assessing extracellular volume status is crucial because a positive daily fluid and salt balance can cause edema and a persistence of a positive daily fluid balance over time is associated with a higher mortality rate in critical ill patients with acute renal injury (Payen, 2008), acute respiratory distress syndrome (Jozwiak, 2013), trauma (Elofson, 2015), subarachnoid hemorrhage (Kissoon, 2015) or sepsis (Acheampong, 2015).

Thus, the technical problem underlying the present invention is the provision of means and methods to provide a fast and reliable way of assessing the extracellular volume status of a subject.

The technical problem is solved by provision of the embodiments provided herein below and as characterized in the appended claims.

### DESCRIPTION OF THE INVENTION

The invention relates to a method for determining the extracellular volume status of a subject, wherein the method comprises determining in a sample obtained from said subject the level of proadrenomedullin (proADM) or a fragment thereof, preferably midregional proadrenomedullin (MR-proADM).

Further, the invention relates to a method for determining the fluid balance, the salt balance and/or the globular volume status of a subject, wherein the method comprises determining in a sample obtained from said subject the level of proadrenomedullin (proADM) or a fragment thereof, preferably midregional proadrenomedullin (MR-proADM).

The present invention solves the above identified technical problem since, as documented herein below and in the appended examples; it was unexpectedly found that there is a surprisingly strong statistical relationship between the level of proadrenomedullin (proADM) or a fragment thereof, preferably midregional proadrenomedullin (MR-proADM) and the extracellular volume status of a subject.

In the appended examples, the results of a clinical study are documented. This clinical study demonstrates that among all biomarkers tested, including cortisol, catecholamine, renin, angiotensin II, aldosterone system (RAAS), vasopressin reflected by CT-pro-AVP (herein also designated as “copeptin”), endothelin reflected by pro-endothelin and natriuretic peptides reflected by proatrial natriuretic peptides (MR-ProANP), erythropoietin (EPO), and pro-adrenomedullin reflected, for example, by MR-pro-ADM, MR-proADM has an unexpectedly strong statistical relationship with the extracellular volume status of the subjects (see, for example, FIG. 2, Table 6). It is documented that this relationship is independent of the type of clinical situation of the patient on day 2, day 5 and day 7 post admission, e.g., patients suffering from an aneurysm (e.g. aneurysmal subarachnoid haemorrhage (SAH)), multiple trauma (e.g., severe trauma without head trauma (PT)), brain injury or head injury (e.g., severe brain trauma (SBT)), or post-operative patients such as post-surgical peritonitis with shock (P); see e.g., FIG. 3.

Therefore, it is shown herein that proADM or a fragment thereof, preferably MR-proADM is a good surrogate for the extracellular volume status of subjects. The appended examples show that high or increased levels of MR-proADM strongly correlate with an increase in salt and/or water in the extracellular volume during the first week after admission of critically ill patients (see, for example, FIG. 2 and Example 1) and that nearly all subjects have a positive fluid balance and/or salt balance, i.e., an increase in extracellular volume. The gains in the extracellular volume are reported as changes in salt balance and changes in water balance of the subjects. It is further shown in the appended examples that the positive fluid balance and/or salt balance does not correlate with, for example, the total blood volume or the plasmatic volume; see, e.g., Example 1.

In order to increase the plasmatic volume, physicians may administer fluid infusions (e.g., crystalloids) to the patient. Undifferentiated fluid handling (e.g., by aggressive fluid therapy) can increase the fluid shift toward the extracellular volume, e.g., into the interstitial space, which, in turn, can cause, for example, interstitial edema. The appended examples demonstrate that the effective volume is the arterial blood volume perfusing tissue.

As shown herein, MR-proADM has a significant relationship with the fluid balance and/or the salt balance (Examples 1 to 4, e.g., FIG. 2). In particular, it is demonstrated that high levels of MR-proADM indicate a volume overload. For example, a high level of MR-proADM, e.g., at least 1 to at least 1.5 nmol/1 of MR-proADM, indicates a fluid overload. Moreover, a high level of MR-proADM and a gain of, for example, at least 27 g to at least 36 g of Na⁺ and/or at least 3 L to at least 4 L of water is a warning sign for the physician to take appropriate actions immediately. Excessive salt and/or fluid balance is considered as a risk factor of morbidity and mortality in critically ill patients (Acheampong et al., 2015). Therefore, the method of the present invention, including measurement of the level of MR-proADM (or the level of proADM or another fragment of proADM), has a high medical potential to quickly, conveniently and reliably determine the fluid balance, salt balance, globular volume status and extracellular volume of a subject and to determine whether a subject has a critical health status.

Moreover, the appended examples document that the assessment of further covariates such as additional markers and parameters improve the discriminative power of the single marker proADM (or a fragment thereof), preferably MR-proADM. For example, MR-proADM alone has a good discriminative power of AUC (“area under the curve”) 0.82 with a variance of 35% for the fluid balance and a discriminative power of AUC 0.79 with a variance of 42% for the salt balance; see, for example, Example 2. The inclusion of further markers and further parameters to MR-proADM such as sex, age, total serum protein, BMI, weight and Hb improves further the prediction of the fluid balance and sodium balance, for example, with ROC curve of 92% (see Tables 13 and 14 and Example 4).

In many intensive care units, nurses systematically measure fluid balance by daily weight or daily calculation of input and output of liquids. These methods are not precise. For example, a weak relationship between weight and fluid balance was found in the appended examples (r²=0.33, see appended Example 1). Conventional markers of extracellular volume described in the prior art such as plasmatic proteins or hemoglobin have a weak relationship with salt and fluid balances as shown herein below (e.g., r²=0.44 for plasmatic proteins and ΔNa⁺ or r²=0.35 for plasmatic proteins and ΔH₂O; r²=0.15 for Hb and ΔNa⁺ or r²=0.24 for Hb and ΔH₂O; see appended Example 1). In the appended examples, measurements of salt and fluid balances required many biological samples and this analysis was always conducted and controlled by two physicians, making this procedure very time and work intensive. It is documented herein that proADM or a fragment thereof, preferably MR-proADM employed in the method of the present invention offers faster and a more exact measure of salt balance and/or fluid balance and thus the extracellular volume status. Therefore, proADM or a fragment thereof, preferably MR-proADM can be employed as an emergency surrogate. In various acute situations, for example in the first days after shock, timing is crucial. A delayed discovery of overload after organ damage such as acute lung injury, abdominal compartment syndrome, and renal insufficiency can have severe and potentially lethal consequences. In the appended examples, it was also surprisingly shown that there is a significant relationship between the sequential organ failure score (SOFA score) (Vincent et al., 1996) and the salt and fluid balance. This prediction model also documents that proADM or a fragment thereof, preferably MR-proADM is an advantageous surrogate and bedside tool.

Therefore, the invention relates to a method for in vitro diagnosis, prognosis, risk assessment, risk stratification, therapy management/control and/or operative control of a disorder or medical condition in a subject, wherein said extracellular volume status of said subject is determined by measuring the level of proADM or a fragment thereof, preferably MR-proADM in whole blood, plasma, serum or urine. The extracellular volume status of said subject can also reflect the sodium retention of said subject.

The present invention has, inter alia, the following advantages over conventional methods: the inventive method is fast, easy to perform and precise for determining the extracellular volume status of a subject, providing a reliable prediction of the extracellular volume status and of positive fluid balance and/or positive salt balance of the subject.

One further advantage of the inventive method is that fluid balance and salt balance correlate with the SOFA score. Therefore, the herein provided method provides a reliable and convenient way to identify a critical subject that is at risk of suffering organ dysfunction or organ failure due to edema caused by a positive fluid and/or salt balance. Further, the inventive method allows the determination of the globular volume status. Example 3 and, in particular, Table 9 document the improved predictive value of the method of the present invention in comparison to the predictive value of globular volume based on Hb alone.

Further, the herein provided inventive method can stratify patients with a positive salt balance and thus can stratify patients that have a sodium retention, which can be a risk factor for hypertension, kidney or heart failure and pulmonary oedema. Such patients may require a different treatment which targets salt mobilization from interstitium to the intravascular system.

It was surprisingly shown in the appended examples that MR-proADM has a significant statistical relationship with the extracellular volume status of a subject. Accordingly, the present invention relates to a method for determining the extracellular volume status of a subject, wherein the method comprises determining in a sample obtained from said subject the level of the marker proADM or a fragment thereof, preferably MR-proADM.

In certain aspects, the present invention relates to the use of the marker midregional proadrenomedullin (MR-proADM) for determining the extracellular volume status of a subject. The peptide adrenomedullin (ADM) was discovered as a hypotensive peptide comprising 52 amino acids, which had been isolated from a human phenochromocytomeby (Kitamura et al., 1993). Adrenomedullin (ADM) is encoded as a precursor peptide comprising 185 amino acids (“preproadrenomedullin” or “pre-proADM”), herein given in SEQ ID NO: 1. ADM comprises the positions 95-146 of the pre-proADM amino acid sequence and is a splice product thereof.

“Proadrenomedullin” (“Pro-ADM”) refers to pre-proADM without the signal sequence (amino acids 1 to 21), i.e. to amino acid residues 22 to 285 of pre-proADM. “Midregional proadrenomedullin” (“MR-proADM”) refers to the amino acids 42-95 of pre-proADM. The amino acid sequence of MR-proADM is given in SEQ ID NO: 2. It is also envisaged herein that a peptide and fragment thereof of pre-proADM or MR-proADM can be used for the herein described methods such as the prediction of the extracellular volume status of a subject. For example, a peptide and fragment thereof can comprise amino acids 22-41 of pre-proADM (PAMP peptide) or amino acids 95-146 of pre-proADM (mature adrenomedullin). A C-terminal fragment of proADM (amino acids 153 to 185 of preproADM) is called adrenotensin. Fragments of proADM peptides or MR-proADM comprise for example 5 or more amino acids. Accordingly, the fragment of proADM may for example be selected from the group consisting of MR-proADM, PAMP, adrenotensin and mature adrenomedullin, preferably herein the fragment is MR-proADM.

It is also envisaged herein that the level of a MR-proADM polypeptide is determined that has a sequence identity of at least 75%, for example, 75%, 80%, 85%, 90%, 91%, 92%, 93%, 94%, 95%, 96%, 97%, 98% or 99% sequence identity as shown in SEQ ID NO: 2, wherein the higher values of sequence identity are preferred. In accordance with the present invention, the terms “sequence identity”, “homology” or “percent homology” or “identical” or “percent identity” or “percentage identity” in the context of two or more amino acid sequences refers to two or more sequences or subsequences that are the same, or that have a specified percentage of amino acids that are the same, when compared and aligned for maximum correspondence over the window of comparison (preferably over the full length), or over a designated region as measured using a sequence comparison algorithm as known in the art, or by manual alignment and visual inspection. Sequences having, for example, 70% to 90% or greater sequence identity may be considered to be substantially identical. Such a definition also applies to the complement of a test sequence. Preferably, the described identity exists over a region that is at least about 15 to about 20 amino acids in length, more preferably, over a region that is at least about 25 to about 45 amino acids in length, most preferably, over the full length. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art.

As used herein, the term “level of the marker proadrenomedullin (MR-proADM) or a fragment thereof” refers to the quantity of the molecular entity of the marker proadrenomedullin or fragments thereof in a sample that is obtained from a subject. In other words, the concentration of the marker is determined in the sample. Hence, the term “level of the marker midregional proadrenomedullin (MR-proADM)” refers to the quantity of the molecular entity of the marker midregional proadrenomedullin (MR-proADM) in a sample that is obtained from a subject. In other words, the concentration of the marker is determined in the sample. As described above, it is also envisaged herein that a fragment of proadrenomedullin (proADM), preferably MR-proADM, can be detected and quantified. Also, fragmemts of MR-proADM can be detected and quantified. Suitable methods to determine the level of proADM or a fragment thereof (preferably MR-proADM) is described herein below in detail Immunoassays in various formats such as for instance sandwich, enzyme-linked immunosorbent assay, luminescent immunoassay, rapid test formats, assays suitable for point-of-care testing and homogeneous assays such as, for example, the Kryptor system (BRAHMS/Thermo Fisher Scientific) can be employed. Moreover, mass spectrometry approaches can be used to detect and quantify proADM or a fragment thereof, preferably MR-proADM or a fragment thereof. The skilled person is aware of assays that are suitable to determine/quantify the herein described markers.

The present invention relates to a method for determining the extracellular volume status of subject. As used herein, the extracellular volume is a part of the body water of a subject. The body water of a subject constitutes as much as about 55-75% of the body weight. The body water of a subject consists essentially of the “extracellular volume” and the “intracellular volume” of a subject; see FIG. 1. As used herein, the “intracellular volume” refers to the cytosol or intracellular fluid (ICF) or cytoplasmic matrix, which is the liquid found inside the cell. Normally, the intracellular volume is about 60% of body water. According to Guyton (Guyton Arthur C., (1991), p. 275), a subject that has a body that contains 40 L of fluid has 25 L of intracellular volume. As used herein, the “extracellular volume” consists essentially of the “total blood volume” and the “interstitial volume”. Normally, the extracellular volume is about 40% of body water. Accordingly, a subject that contains about 40 L of fluid has about 15 L of extracellular volume (Guyton Arthur C., (1991), p. 275). As used herein, the “interstitial volume”, “interstitial fluid” or “tissue fluid” is a solution that bathes and surrounds the tissue cells of multicellular animals. Normally, the interstitial volume is about 28% of body water or about 70% of extracellular volume. As used herein, the “total blood volume” or “intravascular volume” consists essentially of the “plasmatic volume” and “red blood cell volume”. Normally, the total blood volume is about 12% of body water and is composed of about 50% plasma (about 15% of extracellular volume or 6% of body water) and is composed of about 50% globular volume (about 15% of extracellular volume). As used herein, the “red blood cell volume” is also designated “globular volume”. As used herein, the “plasmatic volume” refers to the volume of the “blood plasma” or “plasma”, which is the pale yellow liquid component of blood that normally holds the blood cells in whole blood in suspension; this makes plasma the extracellular matrix of blood cells. It makes up about 55% of the body's total blood volume. It is the intravascular fluid part of extracellular fluid (all body fluid outside of cells). It is mostly water (up to 95% by volume), and contains dissolved proteins (6-8%) (i.e. serum albumins, globulins, and fibrinogen), glucose, clotting factors, electrolytes (Na⁺, Ca²⁺, Mg²⁺, HCO₃—, Cl —, etc.), hormones, and carbon dioxide (plasma being the main medium for excretory product transportation). Plasma also serves as the protein reserve of the human body. It plays a vital role in an intravascular osmotic effect that keeps electrolytes in balanced form and protects the body from infection and other blood disorders. As used herein, the “red blood cell volume” is also designated as the mean corpuscular volume, or mean cell volume (MCV), which is a measure of the average volume of a red blood corpuscle (or red blood cell).

It is documented in the appended examples that the salt balance and/or the fluid balance is calculated to estimate the change in the extracellular volume every day; see appended Example 1. As it is demonstrated in the appended examples, a complete input-output assessment of the previous day is done for the salt and water (content) every day in order to determine the fluid balance and the salt balance of the subject. It is shown therein that the losses of sodium and/or water of the subjects can be measured by determining, e.g., diuresis, ileostomy and ventricular drainage if required. The loss of sodium (Na³⁰ ) can be measured from liquids and can be deducted from the salt contribution; however, measuring the salt balance is in particular difficult. The difference of input water (e.g., enteral nutrition or the sum of crystalloids or colloids infusion of the day) and loss of water is also calculated. Insensitive losses are estimated as a function of the body temperature. In the appended examples, the gain or loss of “sodium” or “Na⁺” (herein also designated as “ΔNa⁺”, “delta sodium” or “sodium balance”) and the gain or loss of water or H₂O (herein also designated as “ΔH₂O” or “fluid balance”) was calculated each day and was summed to the result of the day before as cumulative “fluid balance” or “salt balance”, respectively.

In the appended examples, it was surprisingly demonstrated that the level of MR-proADM in a sample, e.g., a plasma sample, of the subject has a statistical relationship with the fluid balance and/or the salt balance (FIG. 2). Therefore, the invention relates to a method for determining the fluid balance, the salt balance and the extracellular volume status of a subject, wherein the method comprises determining the level of proADM or a fragment thereof, preferably MR-proADM in a sample obtained from said subject, wherein based on the level of proADM or a fragment thereof, preferably MR-proADM the fluid balance and/or the salt balance is determined and wherein said fluid balance and/or salt balance determines/identifies/reflects the extracellular volume status of a subject. It was found that high levels of MR-proADM correlate with a high fluid balance (significant gain of fluid) or a high salt balance (significant gain of salt); see e.g., FIG. 2. Therefore, the level of proADM or a fragment thereof, preferably MR-proADM of the subject can be employed to predict the salt balance and fluid balance of the subject. In other words, proADM or a fragment thereof, preferably MR-proADM can be used as a direct surrogate for the fluid balance and/or salt balance. Accordingly, the term “based on the level of (MR-) proADM” means that the level of (MR-)proADM identifies/predicts/determines the fluid balance and/or salt balance of the subject.

In the appended examples, the salt balance and/or the fluid balance is calculated to estimate the change of or the variation in the extracellular volume. The fluid balance and/or the salt balance is known to correlate with the extracellular volume (Charra et al., 2004). Therefore, the fluid balance and/or the salt balance determine changes in the extracellular volume state. The extracellular volume status refers to the body fluid in the extracellular volume (FIG. 1). The extracellular volume of a subject is about 40% of the body water of the subject. The extracellular volume status of a subject correlates with the fluid balance and/or salt balance of a subject. Therefore, the variation of the fluid balance or salt balance represents the variation of the extracellular volume. Thus, the variation of the fluid balance and/or the salt balance of the subject allows the estimation/determination of the extracellular volume status of the subject. In other words, proADM or a fragment thereof, preferably MR-proADM is a direct surrogate for the fluid balance and/or the salt balance of the subject and hence it indicates the extracellular volume status of the subject. As used herein, the “fluid balance” refers to the “variation of water”, “change of water”, “delta water” or “ΔH₂O” of a subject. In other words, the “fluid balance” is the difference between the input and output of “fluid” or “water” of a subject. In preferred aspects of the invention, the fluid balance is the difference between input and output of fluid/water of a subject. In even more preferred aspects, the fluid balance is the cumulative fluid balance reflecting the difference between input and output of fluid/water during the hospitalization of the subject. As used herein, the term “during the hospitalization” or “per hospitalization” means the time period in which the patient is in a critical health situation. Thus, as used herein the hospitalization of the subject can mean the time period in which the subject enters the ICU until the critical situation and/or the symptom(s) is alleviated. Alternatively, this term relates to the time period in which the patient has accumulated a positive fluid balance, e.g., of 4 L, or a positive salt balance, e.g., of 36 g. In other words, a time period is meant in this aspect in which the subject has accumulated a gain of e.g., 4 L of fluid or 36 g of salt. In the appended examples, the fluid balance and the salt balance was calculated every day. Therefore, the fluid balance is the difference between input and output of fluid/water of a subject within the first day (per day).

In preferred aspects, the fluid balance is the cumulative fluid balance, which is the difference between input and output of fluid/water of a subject within, the first two days, even more preferred within the first five days, most preferred within the first week, i.e., the difference of input and output of fluid/water of a subject after 7 days. It is herein understood that a gain/increase of water of a subject refers to a subject that has more water compared to an earlier time point (e.g., one day before) as the output of fluid/water is less than the gain of water. It is herein understood that a loss/decrease of water of a subject refers to a subject that has a less water compared to an earlier time point (e.g., one day before). It is herein understood that no change or no significant change of water of a subject refers to a subject that has an identical or similar water content compared to an earlier water content (e.g., one day before). In preferred aspects, MR-proADM is determined at several time points, e.g., at day 0 (“D0”), day 2 (“D2”), day 5 (“D5”) and/or day 7 (“D7”) after admission into a health care unit, particularly, into intensive care. It is herein understood that the levels of the marker and/or parameter can be determined at any time and at any interval, e.g., hourly or daily (e.g., at admission D0, and then at day 1 (D1), day 2 (D2), day 3 (D3), day 4 (D4), day 5 (D5), day 6 (D6) and/or day 7 (D7) after admission of the subject into an ICU or the like) or a combination thereof.

It is shown in the appended examples that the level of MR-proADM correlates with the fluid balance of a subject; see, FIG. 2. It is understood herein that high levels of proADM or a fragment thereof, preferably MR-proADM indicate a gain/increase of fluid of a subject. As used herein, a subject that has a “positive fluid balance” refers to a subject in which the fluid gain is higher than the fluid loss. Therefore, the subject has an imbalance of fluid/water input and output. Accordingly, the subject with a positive fluid balance accumulates water/fluid in the body. Thus, the subject gains weight. In other words, a subject that has an increase of the water content or a gain of water has positive fluid balance. For example, subjects that are treated with liquid infusions can have a fluid shift of fluid/water out of the vasculature. This fluid can shift toward the extracellular volume, e.g., the interstitial volume of a subject. For example, extracellular volume overload exceeding 10 L after 3 days of a resuscitation patient has been shown to be trapped in the body and needed 3 weeks to be excreted (Chappell et al.; A rational approach to perioperative fluid management, Anesthesiology; 2008, 109:723-40). Anatomical losses are considered to be a physiologic phenomenon at a pathologic amount, i.e., pathologic fluid accumulation within the interstitial space (Chappell, loc. cit.). Physiologic fluid shifting with an intact vascular barrier from the vessels toward the interstitial space can be considered to contain only small amounts of protein and primarily small molecules. When this shift is quantitatively managed by the lymphatic system, a physiologic shift does not cause edema, such as interstitial edema. However, overwhelming the lymphatic system, e.g., via excessive application of liquid infusions such as crystalloids, can cause edema. There are also non-anatomical third space losses representing a fluid compartment functionally and anatomically separated from the interstitial volume. Losses toward this third space can be fluid accumulations caused by, for example, surgical procedures or trauma in spaces normally containing no or little fluid. For example, third space losses can be toward the peritoneal cavity, the bowel, and traumatized tissues.

In certain aspects of the present invention, a positive fluid balance of 3 to 4 kg gain of weight (during the hospitalization, e.g., within the first day, preferably within the first two days, even more preferred within the first five days, most preferred within the first week) is considered as the threshold where mortality and morbidity increase. Hence, in certain aspects of the present invention, a fluid gain of at least 3L, preferably, of at least 4 L is considered as critical. A gain of fluid in the extracellular volume of at least 3 L, preferably, of at least 4 L is considered as critical. The gain of fluid, which is considered as critical, is also dependent on the patient characteristics such as sex, age or weight of the subject. For example, the body water of an adult female is 5 to 10% lower than that the body water of an adult male. Thus, it is herein understood that a patient that has a lower weight (e.g., a female) can react more sensitively to fluid and/or salt gain. Further, the distribution of the fluid in the fluid compartments is dependent on the age of the subject, e.g., it decreases from 75% of a newborn to 55% of an adult. Thus, a lower fluid gain, e.g., 3L or less of fluid, can already have severe consequences in a female or old subject. On the other hand, a subject that has a higher weight (e.g., a male) might not be as sensitive to fluid and/or salt gain as said light subject. Therefore, the mortality risk can decrease in such subjects.

In another aspect, a positive fluid balance of at least 4L is considered as critical. In other words, a positive fluid balance of at least 4L indicates that the subject has an extracellular volume status that is considered as critical. In particular, it is documented in the appended examples that a high gain of water, for example, at least 4 L of water, is a warning sign and indicates a critical extracellular volume status and thus a critical subject. Endothelial damage and/or salt retention can be responsible for the increase of fluid balance.

As used herein, a subject that has a “negative fluid balance” refers to a subject in which the fluid loss is higher than the fluid gain. Accordingly, the subject looses water or fluid and thus looses weight. In other words, a subject that has a decrease of the water content or a loss of water has negative fluid balance. As used herein a subject that has identical or similar water content is referred to a subject that has an “identical or similar fluid balance”. Such a subject has a balanced fluid management and thus the fluid balance is in balance or normal. In other words, the fluid/water input is identical or similar to the fluid/water output. In other words, the subject has a normal fluid balance.

In the context of the present invention, the fluid/water balance of a subject can, for example, be increased by intravenous therapy including, for example, volume expanders. In particular preferred aspects, a gain of water of a subject refers to a subject that has more water/fluid compared to the water/fluid that was determined at an earlier time point, wherein said subject that has more water/fluid is referred to a subject that has a positive fluid balance. It is shown in the appended examples that a high level of MR-proADM, e.g., at least 1 nmol/1, indicates a gain of water, i.e., a positive fluid balance and thus an increased extracellular volume status, wherein said extracellular volume status is considered as critical. In other words, a positive fluid balance of at least of at least 4L indicates that the subject has an extracellular volume status that is considered as critical.

The invention relates to the herein provided method, wherein based on the level of proADM or a fragment thereof, preferably MR-proADM the salt balance is determined and wherein said salt balance determines/identifies the extracellular volume status. As used herein, the “salt balance” or “sodium balance” refers to the “variation of sodium”, “change of sodium”, “delta sodium” or “ΔNa⁺”. The “salt balance” is the difference between the input and output of “salt” or “sodium” of a subject. In preferred aspects of the present invention, the salt balance is the difference between input and output of fluid/water of a subject. In even more preferred aspects, the salt balance is the cumulative salt balance reflecting the difference between input and output of salt/sodium during the hospitalization of the subject. In certain aspects, the salt balance is the difference between input and output of salt/sodium of a subject within the first day per day. In preferred aspects, the salt balance is the difference between input and output of salt/sodium of a subject, within the first two days, even more preferred within the first five days, most preferred within the first week, i.e., the difference after 7 days. In the appended examples, it was surprisingly demonstrated that the salt balance has a statistical relationship with the level of MR-proADM in a sample, e.g., a plasma sample of the subject (FIG. 2B). It is further shown in the appended examples that the fluid balance of a subject is statistically related to the salt balance; see, Example 1. It is herein understood that a gain/increase of salt, e.g., sodium, of a subject refers to a subject that has higher amount/content of salt, e.g., sodium. As used herein a subject that has higher amount/content of salt is referred to a subject that has a positive salt balance. Hence, a subject with a “positive salt balance” means herein a subject that has a salt gain that is higher than the salt loss of the subject. Therefore, the subject has an imbalance of salt/sodium input and output. Accordingly, the subject with a positive salt balance accumulates salt/sodium in the body, e.g., salt retention. In other words, a subject that has an increase of the salt/sodium amount/content or a gain of sodium/salt has positive sodium balance. As used herein, “sodium retention” or “salt retention” can be indicative for kidney or heart failure. Salt retention can result in fluid/water retention and increased blood volume, increased blood pressure and inflammation. Without being bound by theory, inflammation can cause salt retention. As MR-proADM has a strong statistical relationship with salt and/or fluid balance, MR-proADM (or proADM or another fragment thereof) can be employed as a prognosis marker for inflammation (or vascular damages and permeability provoked by inflammation).

As used herein, a subject that has a “negative salt balance” refers to a subject in which the salt loss, e.g., sodium, is higher than the salt gain. Accordingly, the subject looses salt or fluid and thus looses weight. In other words, a subject that has a decrease of the salt content or a loss of salt has negative salt balance. As used herein a subject that has identical or similar salt content is referred to a subject that has an “identical or similar salt balance”. Such a subject has a balanced salt management and thus the salt balance is in balance or normal. In other words, the salt/sodium input is identical or similar to the salt/sodium output. In other words, the subject has a normal salt balance.

In one aspect, a positive salt balance means that the subject has a higher salt content and thus an increased extracellular volume status compared to an earlier time point. It is shown in the appended examples that a high level of MR-proADM, e.g., more than 1 nmol/1, indicates a gain of salt, a positive salt balance and thus an increased extracellular volume status, wherein said increased extracellular volume state is considered as critical. In particular, it is documented in the appended examples that a high gain of salt, for example at least 27 g, preferably at least 36 g of sodium, is a warning sign and indicates a critical patient. Hence, in certain aspects of the present invention, a salt gain of at least about 27 to at least about 36 g is considered as critical. In preferred aspects of the present invention, a positive salt balance of at least 36 g is considered as critical. In other words, a positive salt balance of at least of at least 27 g, or preferably of at least 36 g indicates that the subject has an extracellular volume status that is considered as critical.

In certain aspects of the invention, the method provided herein determines the globular volume status of a subject. In particular, the herein provided method allows the determination whether the globular volume of a subject is under 20 ml/kg. The globular volume under 20 ml/kg, or preferably a globular volume under 15 ml/kg, indicates a critical globular volume status. In certain aspects, the globular volume of a subject under 20 ml/kg is predictive for a subject with a critical extracellular volume status, wherein said critical extracellular volume status indicates a critical health status of the subject. Therefore, a globular volume below 20 ml/kg indicates that the subject has a positive fluid balance, wherein said positive fluid balance indicates a critical extracellular volume status.

The method provided herein determines the globular volume status of a subject, wherein the method comprises determining the level of proADM or a fragment thereof, preferably MR-proADM in the sample, the level of hemoglobin in the sample, body mass index of the subject, sex of the subject, age of the subject, the level of the total serum protein in the sample and optionally weight of the subject.

In the appended examples, it is documented that the inclusion of further markers or parameters in the statistical analysis improves the predictive power of proADM or a fragment thereof, preferably MR-proADM; see e.g., Examples 1 to 4. The statistical analysis surprisingly found consensus model(s) including MR-proADM that has a significant relationship with the extracellular volume status of a subject. As used herein, a consensus model includes more than one marker and parameter and based on said consensus model the fluid balance and/or salt balance and/or extracellular volume status of a subject can be determined. In other words, in certain aspects, the invention relates to a method wherein a panel (or multi-panels) of marker(s) and parameter(s) are determined. Therefore, in the context of the invention further parameters and/or marker can be determined. In other words, the method according to the present invention can be conducted in combination with other markers, parameters and/or methods. This means that the measurement methods according to the present invention can be conducted particularly advantageously as multi-parameter diagnostic. Hereby, at least one further marker, preferably chosen from the group of vasodilators is determined additionally.

In certain aspects of the present invention, the herein provided method comprises determining the level of a least one further marker selected from the group consisting of hemoglobin, total serum protein, renin, pro-atrial natriuretic peptide (proANP), C-terminal pro-arginine-vasopressin (CT-proAVP) protein, erythropoietin, angiotensin II, aldosterone, cortisol, adrenaline, epinephrine, catecholamines and pro-endothelin-1 (pro-ET-1).

In certain aspects, the invention relates to the use of one further marker selected from the group consisting of hemoglobin, total serum protein, renin, pro-atrial natriuretic peptide (proANP), C-terminal pro-arginine-vasopressin (CT-proAVP) protein, erythropoietin, angiotensin II, aldosterone, cortisol, adrenaline, epinephrine, catecholamines and pro-endothelin-1 (pro-ET-1).

In certain preferred aspects, the herein provided method further comprises determining the level of the marker “hemoglobin” (herein also designated as “haemoglobin”). “Hemoglobin” or “Hb” is the iron-containing oxygen-transport metalloprotein in the red blood cells of vertebrates. The Hb concentration can be measured in the context of conventional blood tests, usually as part of a complete blood count. Normal Hb concentrations are for: men: 13.8 to 18.0 g/dL (138 to 180 g/L, or 8.56 to 11.17 mmol/L); women: 12.1 to 15.1 g/dL (121 to 151 g/L, or 7.51 to 9.37 mmol/L); children: 11 to 16 g/dL (111 to 160 g/L, or 6.83 to 9.93 mmol/L); or pregnant women: 11 to 14 g/dL 9.5 to 15(usual value during pregnancy)(110 to 140 g/L, or 6.83 to 8.69 mmol/L). In the context of the present invention, low hemoglobin means that a person's hemoglobin level, is below the lowest limits of normal for their age and sex (see above normal range of values). For example, a 19 year old male has a low hemoglobin level, if the detected blood value is below 13.6 g/dl. In the context of the present invention, high hemoglobin levels mean that measured hemoglobin levels are above the upper limits of normal for the age and sex of the person (see above normal values). For example, a 19 year old male that has a detected hemoglobin level of above 18.2 g/dl has a high hemoglobin level.

As used herein, “total serum protein” refers to the total amount of protein in the blood. In preferred aspects, the total serum protein refers to the total amount of protein in blood serum or blood plasma. The “total serum protein” is measured in routine tests and is used in ICU and other medical services. The two major protein components in the serum or plasma are albumins and globulins. Globulin is made up of different proteins called alpha, beta, and gamma types. A test for total serum protein reports separate values for total protein, albumin, and globulin. The total serum protein can, for example, be determined by the biuret reagent or by a refractometry method. Hypoproteinemia results from deficient synthesis due to hepatic failure, malnutrition, or from renal loss. Elevation of serum protein concentration has 2 principal causes: dehydration, in which there is less water in the body and the blood volume decreases. The most commonly overproduced proteins are immunoglobulins, the levels of which can be elevated in infections and in hematological neoplasms. The normal range of total serum protein is about 60 to about 80 g/l.

As used herein, “renin” or “angiotensinogenase”, is an enzyme that participates in the body's renin-angiotensin aldosterone system (RAAS) that mediates extracellular volume (i.e., that of the blood plasma, lymph and interstitial fluid), and arterial vasoconstriction. Thus, it regulates the body's mean arterial blood pressure. The level of renin is preferably measured in the plasma or serum of a subject.

As used herein, “pro-atrial natriuretic peptide” or “proANP” refers to the pro-hormone comprising 128 amino acids. As used herein, a peptide comprising 28 amino acids (99-126) of the C-terminal section of a pro-hormone comprising 128 amino acids (proANP) is referred to as the actual hormone ANP. Upon release of ANP from its pro-hormone proANP, an equimolar amount of the remaining larger partial peptide of proANP, the N-terminal proANP, consisting of 98 amino acids (NT-proANP; proANP (1-98)) is released into circulation. As NT-proANP possesses a significantly greater half life time and stability NT-proANP can be used as laboratory parameter for diagnosis, follow-up and therapy control; see, for example, Lothar Thomas (Editor), Labor and Diagnose, 5^{th} expanded ed., sub-chapter 2.14 of chapter 2, Kardiale Diagnostik, pages 116-118, and WO 2008/135571. The level of proANP is preferably measured in the plasma or serum of a subject.

As used herein, endothelin-1 is derived from a larger precursor molecule named pro-endothelin-1. pro-endothelin-1 can be proteolytically processed into various fragments as described (EP 2 108 958 Al; Proteolytic processing pattern of the endothelin-1 precursor in vivo. Peptides. 2005 Dec; 26(12):2482-6.). These fragments are subject to proteolytic degradation in the blood circulation, which can happen quickly or slowly, depending on the type of fragment and the type and concentration/activity of proteases present in the circulation. Thus, according to the present invention the level of any of these fragments of at least 12 amino acids may be measured, preferably fragments of at least 20 amino acids, more preferably of at least 30 amino acids. Preferably, C-terminal pro-ET-1 (CT-proET-1) or a fragment thereof may be measured. The level of endothelin-1 is preferably measured in the plasma or serum of a subject.

As used herein, “vasopressin” refers to “AVP”. Vasopressin is a powerful vasoconstrictor. Assaying of its prohormone has been examined as a prognostic and diagnostic factor for cases of diabetes insipidus. Vasopres sin or antidiuretic hormone (ADH) is one of the keys to regulating body water and water balance. Its secretion, which is partly linked to stress, causes arterial pressure to rise and water to be absorbed, risking the onset of hyponatraemia. However, ADH is unstable. Moreover, its concentration is dependent on its bonds to platelets and is therefore labile. The C-terminal portion of the prohormone “CT-proAVP”, is a more stable precursor of ADH and its plasma concentration reflects ADH secretion (Struck, 2005, Morgenthaler, 2007). As used herein, the C-terminal portion of the prohormone is referred to as “CT-proAVP” or “copeptin”. Increased plasma levels after septic shock or haemorrhage correlates with blood osmolarity and mortality (Morgenthaler, 2007). The level of CT-proAVP is preferably measured in the plasma or serum of a subject.

Angiotensin I is converted to angiotensin II through removal of two C-terminal residues by the enzyme angiotensin-converting enzyme (ACE), primarily through ACE within the lung (but also present in endothelial cells and kidney epithelial cells). Angiotensin II acts as an endocrine, autocrine/paracrine, and intracrine hormone. The level of angiotensin II is preferably measured in the plasma or serum of a subject.

In certain aspects, the herein provided method further comprises determining at least one parameter of the subject selected from the group consisting of body mass index, weight, age, sex, IGS II, lactate, sodium intake, liquid intake and patient group. In certain preferred aspects, the herein provided method further comprises determining at least one parameter of the subject selected from the group consisting of body mass index, weight, age and sex.

As used herein, the body mass index (BMI) is a value derived from the mass (weight) and height of the subject. The BMI is defined as the body mass of the subject, i.e., weight, divided by the square of the body height of the subject, and is universally expressed in units of kg/m², resulting from weight in kilograms and height in metres. The BMI may also be determined using a table or chart (reference values), which displays BMI as a function of mass and height using contour lines or colors for different BMI categories, and may use two different units of measurement. The BMI is an attempt to quantify the amount of tissue mass (muscle, fat, and bone) in an individual, and then categorize that person as underweight, normal weight, overweight, or obese based on that value. Commonly accepted BMI ranges are underweight: under 18.5, normal weight: 18.5 to 25, overweight: 25 to 30, obese: over 30. In certain aspects of the invention, the BMI is determined at day 0, e.g., at the patient admission.

As used herein, the “weight” refers to the mass of the subject in kg (see BMI). In certain aspects of the invention, the weight is determined at day 0, e.g., at the patient admission. In the context of the present invention, a normal body weight can be theoretically calculated according to the Devin Formula or the Hamwi method. According to the Hamwi method, the ideal body weight of a man is 48 kg plus 2.7 kg for every 2.54 cm over 1.5 m. For women, it is 45 kg plus 2.3 kg for every 2.54 cm over 1.5 m. Values below or above these normal values increase the risk to be a critical subject.

As used herein, “age” refers to the length of time that an individual has lived in years. In preferred aspects, the parameter is weighted in the method by adding the age squared and cubed, i.e., age² and age³.

As used herein, “IGS II” (that is the abbreviation of “Indice de Gravite Simplifie”) or “SAPS II” (that is the abbreviation of “Simplified Acute Physiology Score II”) relates to a system for classifying the severity of a disease or disorder (see Le Gall JR et al., A new Simplified Acute Physiology Score (SAPS II) based on a European/North American multicenter study. JAMA. 1993; 270(24):2957-63.). The “IGS II score” is made of 12 physiological variables and 3 disease-related variables. The point score is calculated from 12 routine physiological measurements, information about previous health status and some information obtained at admission to the ICU. The IGS II can be determined at any time, preferably, at day 2. The “worst” measurement is defined as the measure that correlates to the highest number of points. The SAPS II score ranges from 0 to 163 points. The classification system includes the followings parameters: Age, Heart Rate, Systolic Blood Pressure, Temperature, Glasgow Coma Scale, Mechanical Ventilation or CPAP, PaO2, FiO2, Urine Output, Blood Urea Nitrogen, Sodium, Potassium, Bicarbonate, Bilirubin, White Blood Cell, Chronic diseases and Type of admission. There is a sigmoidal relationship between mortality and the total SAPS II score. The mortality of a subject is 10% at a SAPSII score of 29 points, the mortality is 25% at a SAPSII score of 40 points, the mortality is 50% at a SAPSII score of 52 points, the mortality is 75% at a SAPSII score of 64 points, the mortality is 90% at a SAPSII score of 77 points (Le Gall loc. cit.).

As used herein, the “liquid intake” refers to the fluid intake of the subject within a given time, e.g., within 24 hours. For example, the fluid intake of a patient or a subject can be a fluid infusion or fluid resuscitation. Preferably, the liquid intake is determined at day 0, in other words, at or after patient admission.

As used herein, the “sex” of a subject refers to the biological gender of the subject, wherein the subject is either a male or a female.

As used herein, “sodium intake” refers to the total amount of salt, or preferably sodium, e.g., sodium chloride, an organism receives from external sources such as nutrition (food and liquids), or liquid infusion. Preferably, the sodium intake is determined at day 0, in other words, at or after patient admission.

As used herein, “lactate” or “max.lactate” refers to the maximal lactate concentration measured in the blood. Normally, the lactate concentration is assessed daily or even more often. The lactate concentration in the blood can be determined by lactate oxidase spectrophotometric methods.

As used herein, the “total blood volume”, “TBV” or “TV” can be measured employing red blood cells marked with chrome 51 (Cr₅₁). The total blood volume can be measured at any time, particularly, between day 1 to day 3, e.g., at day 3; and/or between day 6 to day 10, e.g., at day 10. It is envisaged herein that the patient's own blood is radio-labeled with chrome 51 (Cr₅₁) and radioactively labeled red blood cells are selected. A known quantity of radioactively labeled red blood cells is then re-injected into the total blood circulation. For example, two samples can be performed in an arterial line at two time points, e.g., at 10 and 30 minutes. In order to deduce the total blood volume (TBV) in mL or mL/kg, the radioactivity of the two samples is measured and the weight of the patient is determined (Gore et al., 2005). The haematocrit number and the measured total blood volume define the red blood cells volume (RBCV) in (mL or mL/kg) and plasmatic volume (PV) in mL or mL/kg. The normal values (±20%) are about 72±14 mL/kg for TBV, about 32±6 mL/kg for RBCV and about 40±8mL/kg for PV (Gore et al., 2005).

The “plasmatic volume” or “PV” can be measured employing iodine-125 (PVI₁₂₅). The plasmatic volume can be measured at any time, e.g. day 7. In the appended examples, PVL₁₂₅ is measured at day 7. A defined amount of radio-labeled albumin with iodine 125 (I₁₂₅) is injected to the patient and samples are collected at several time points, e.g., at 10 min, 30 minutes and 2 hours after injection (Fairbanks et al., 1996). The normal value of PV measured by I₁₂₅ is about 45±10 mL/kg (Gore et al., 2005). In general, the plasmatic volume measured by I₁₂₅-albumin is slightly larger than the plasmatic volume measured by Cr₅₁-red blood cells because of a greater volume of the distribution of albumin than that of the red blood cells (Gore et al., 2005).

As used herein, “patient group” or “group” means that the subjects are sorted according to their disease pattern or medical picture. As used in the appended examples, the subjects are sorted in 4 groups, i.e., severe brain trauma (SBT), aneurysmal subarachnoid haemorrhage (SAH), severe trauma without head trauma (PT) and post-surgical peritonitis with shock (P).

In certain aspects, the method determines the extracellular volume status of a subject, wherein the method comprises determining at least one marker and/or parameter selected from the group consisting of proADM or a fragment thereof, preferably MR-proADM, sex, hemoglobin, total serum protein, IGS II score, fluid intake and sodium intake. In the appended examples, it is demonstrated that a random forest analysis can be used to select the combination of markers and parameters yielding the lowest error. It is herein surprisingly shown that the best model for prediction of the extracellular volume status of a subject is achieved by using the level of MR-proADM in combination with body mass index, weight, age (age² and age³) and sex of the subject, hemoglobin and total serum protein. Hence, in most preferred aspects of the invention, the herein provided method comprises determining the level of proADM or a fragment thereof, preferably MR-proADM in the sample, the body mass index, the weight, the age, the sex of the subject, the level of hemoglobin in the sample and the level of the total serum protein in the sample. As it is demonstrated in the appended Example 4, the markers such as MR-proADM, total serum protein and hemoglobin have a good prediction power, for example an AUC of 0.94 for the fluid balance; see e.g. Table 13 and 14. The addition of the parameters such as body mass index, weight, age and sex of the subject to the markers improves the AUC, for example, to 0.95 for the fluid balance. The parameters alone such as body mass index, weight, age and sex of the subject have, for example, an AUC of 0.88 for the fluid balance. Hence, in certain aspects of the invention, the herein provided method comprises determining the body mass index, weight, age, sex of the subject. In preferred aspects of the invention, the herein provided method comprises determining the level of proADM or a fragment thereof, preferably MR-proADM in the sample, the level of hemoglobin in the sample and the level of the total serum protein in the sample. In most preferred aspects of the invention, the herein provided method comprises determining the level of proADM or a fragment thereof, preferably MR-proADM in the sample, body mass index, weight, age, sex of the subject, the level of hemoglobin in the sample and the level of the total serum protein in the sample.

As it is documented in the appended examples, in particular, in Examples 3 and 4, different combinations of markers and parameters might be used to determine the extracellular volume status of a subject. In certain aspects of the invention, the method provided herein determines the fluid balance of a subject, wherein the method comprises determining the level of proADM or a fragment thereof, preferably MR-proADM in the sample, body mass index, weight, age, sex of the subject, the level of hemoglobin in the sample, the level of the total serum protein in the sample, the IGS II score and the fluid intake of the subject. In certain aspects of the invention, the method provided herein determines the salt balance of a subject, wherein the method comprises determining the level of proADM or a fragment thereof, preferably MR-proADM in the sample, body mass index, weight, age, sex of the subject, the level of hemoglobin in the sample, the level of the total serum protein in the sample, the level of sodium intake in the sample, the IGS II score and the fluid intake of the subject. As it is shown in the appended Example 3, the absence of the parameters IGS II and liquid intake has a minor effect on the statistical analysis with only a loss of 2 to 3% of r² and no effect on the AUC; see, e.g., Table 6. Thus, in certain other aspects of the invention, the herein provided method comprises determining the level of proADM or a fragment thereof, preferably MR-proADM in the sample, body mass index, weight, age, sex of the subject, level of hemoglobin in the sample and level of total serum protein in the sample.

In certain aspects of the present invention, the method comprises determining at least one further marker and/or parameter of the subject selected from the group consisting of the level of proANP in the sample, the level of total blood volume, the level of haematocrit in the sample, the level of red blood cells volume, the level of plasmatic volume, the level of total urine volume, the level of angiotensin II in the sample, the patient group of the subject, the level of cortisol in the sample, number of endothelial stem cells in the blood, the level of catecholamines in the sample, full blood ionogram of the subject, urinary ionogram of the subject, blood osmolarity of the subject, urine osmolarity of the subject, blood sugar of the subject, the level of pro-endothelin-1 (pro-ET-1) in the sample, the level of CT-proAVP in the sample, the level of aldosterone in the sample, the level of lactate in the sample, Acute Physiology and Chronic Health Evaluation II (APACHE II) of the subject, World Federation of Neurosurgical Societies (WFNS) grading of the subject, and Glasgow Coma Scale (GCS) of the subject.

It is documented in the appended examples that there is a significant statistical relationship between the sequential organ failure score of the subject (SOFA score) and the fluid balance and/or salt balance; see Examples 1 and 3 and FIG. 4. MR-proADM correlates with the fluid balance and/or salt balance. Hence, in certain aspects, the sequential organ failure score (SOFA score) is determined based on the level of proADM or a fragment thereof, preferably MR-proADM. In other words, proADM or a fragment thereof, preferably MR-proADM is used as a surrogate marker for the SOFA score.

In certain other aspects of the present invention, the sequential organ failure assessment score (SOFA score) is determined based on the fluid balance and/or salt balance. It is shown in the appended examples, that the inclusion of further parameters such as age, BMI and sex improve the predictive power to determine the SOFA score; see FIG. 5. Thus, in certain aspects, the herein provided method determines the SOFA score based on the fluid balance and/or salt balance, wherein the method further comprises determining at least one parameter consisting of age, body mass index and sex.

Furthermore, the invention relates to a method, wherein said method comprises:
(a) determining a level of proADM or a fragment thereof, preferably MR-proADM, in a sample of a subject, and
(b1) comparing said level of proADM or said fragment thereof, preferably MR-proADM, to reference data corresponding to said level of proADM or said fragment thereof, preferably MR-proADM, of at least one reference subject; or
(b2) comparing said level of proADM or said fragment thereof, preferably MR-proADM, to data corresponding to said level of proADM or said fragment thereof, preferably MR-proADM, of the same subject obtained from prior analysis;
(c) identifying the fluid balance and/or salt balance of said subject based on the comparison step (b); and
(c) identifying the globular volume status and/or the extracellular volume status based on step (c).

In other words, the invention relates to the herein provided method, wherein said method comprises:
(a) determining a level of proADM or a fragment thereof, preferably MR-proADM, in a sample of a subject, and
(b1) comparing said level of proADM or said fragment thereof, preferably MR-proADM, to a reference level of proADM or said fragment thereof, preferably MR-proADM, of at least one reference subject or a population of reference subjects; or
(b2) comparing said level of proADM or said fragment thereof, preferably MR-proADM, to a reference level of proADM or said fragment thereof, preferably MR-proADM, of the same subject obtained from prior analysis;
(c) identifying the fluid balance and/or salt balance of said subject based on the comparison step (b); and
(c) identifying the globular volume status and/or the extracellular volume status

Furthermore, the invention relates to an in vitro method, wherein said method comprises:
(a) determining a level of proADM or a fragment thereof, preferably MR-proADM, in a sample and (a) level(s) of at least one further marker and/or at least one further parameter of a subject, and
(b1) comparing said level of proADM or said fragment thereof, preferably MR-proADM, and level(s) of at least one further marker and/or parameter to reference data corresponding to said level of proADM or said fragment thereof, preferably MR-proADM, and said level(s) of at least one further marker and/or parameter of at least one, reference subject; or
(b2) comparing said level of proADM or said fragment thereof, preferably MR-proADM, and level(s) of at least one further marker and/or parameter to data corresponding to said level of proADM or said fragment thereof, preferably MR-proADM, and said level(s) of at least one further marker and/or parameter of the same subject obtained from prior analysis; and
(c) identifying the fluid balance and/or the salt balance of said subject based on the comparison step (b1) or (b2); and
(d) identifying the globular volume status and/or the extracellular volume status based on step (c).

The invention also relates to an in vitro method, wherein said method comprises:
(a) determining the level of proADM or a fragment thereof, preferably MR-proADM, in a sample of a subject, the body mass index of the subject, the weight of the subject, the age of the subject, the sex of the subject, the level of hemoglobin in the sample and the level of the total serum protein in the sample; and
(b1) comparing said level of proADM or said fragment thereof, preferably MR-proADM, the body mass index, the weight, the age, the sex, the level of hemoglobin and the level of the total serum protein to reference data corresponding to said levels of said markers and to said parameters of at least one reference subject; or
(b2) comparing said level of proADM or said fragment thereof, preferably MR-proADM, the body mass index, the weight, the level of hemoglobin and the level of the total serum protein to data corresponding to said levels of said markers and to said parameters of the same subject obtained from prior analysis;
(c) identifying the fluid balance and/or the salt balance of said subject based on the comparison step (b1) or (b2); and
(d) identifying the globular volume status and/or the extracellular volume status based on step (c).

In certain aspects of the invention, the term “comparing said level of proADM or a fragment thereof to reference data” or “comparing said level of proADM or a fragment thereof to reference data corresponding to said level of proADM or said fragment thereof of at least one reference subject” means that the level of proADM or said fragment thereof is determined as described herein and the level of proADM or said fragment thereof is compared to the level(s) of proADM or said fragment thereof determined in at least one reference subject. Accordingly, the term “comparing said level of MR-proADM to reference data” or “comparing said level of MR-proADM to reference data corresponding to said level of MR-proADM of at least one reference subject” means that the level of MR-proADM is determined as described herein and the level of MR-proADM is compared to the level(s) of MR-proADM determined in at least one reference subject. In these aspects, the reference data correspond to the levels of proADM or a fragment thereof, preferably MR-proADM determined in these reference subjects. In other words, said level of proADM or a fragment thereof, preferably MR-proADM is compared to a reference level of proADM or a fragment thereof, preferably MR-proADM of at least one reference subject or a population of reference subjects. The reference level is commonly referred to herein as reference data. The reference data can contain more levels/values corresponding to, for example, further marker and/or parameter. In preferred aspects of the invention, the term “comparing said level of proADM or said fragment thereof, preferably MR-proADM, and level(s) of at least one further marker and/or parameter to reference data corresponding to said level of proADM or said fragment thereof, preferably MR-proADM, and said level(s) of at least one further marker and/or parameter of at least one reference subject” means that the level of proADM or said fragment thereof, preferably MR-proADM, is determined and at least one level of at least one further marker and/or at least one further parameter is determined and that the level of proADM or said fragment thereof, preferably MR-proADM, is compared to a corresponding level of proADM or said fragment thereof, preferably MR-proADM, of at least one reference subject and that the level(s) of the at least one further marker and/or at least one further parameter is compared to the corresponding level(s) of the at least one further marker and/or at least one further parameter of the at least one reference subject. In certain aspects, the reference data corresponds to the levels of the proADM or said fragment thereof, preferably MR-proADM, and the level(s) of at least one further marker and/or parameter determined in the reference subject(s). The level of proADM or said fragment thereof, preferably MR-proADM, and the level(s) of at least one further marker and/or parameter of the subject to be tested are compared to the reference data of such reference subjects.

In another aspect of the invention, the reference data correspond to the levels of proADM or a fragment thereof, preferably MR-proADM, the body mass index, the weight, the age, the sex, the level of hemoglobin and the level of the total serum protein determined in the reference subjects. The level of proADM or a fragment thereof, preferably MR-proADM, the body mass index, the weight, the age, the sex, the level of hemoglobin and the level of the total serum protein of the subject to be tested are compared to the reference data of such reference subjects.

In certain aspects of the invention, a reference subject may be a healthy subject, e.g., a subject having a normal extracellular volume status. In a further aspect of the invention, a reference subject may be a subject suffering from a disease or disorder. The population of healthy or diseased/disordered reference subjects consists essentially of healthy subjects or subjects suffering from a disease or disorder, respectively. A population of reference subjects is a population of subjects comprising 1 to 200 or more reference subjects.

In particular, the healthy subject(s) do(es) not suffer edema, brain damage, post-aneurysm rupture, head injury, neurological impairment, multiple traumatic injuries, post-operative, organ failure, disregulated lymphatic flow activity, kidney dysfunction, cardiac dysfunction, and/or disease associated with disordered fluid balance. In particular, the healthy subject(s) does not suffer from aneurysm, multiple trauma, brain injury and/or head injury and is/are not (a) post-operative patient(s).

In particular, the reference subject or the population of reference subjects suffering from a disease or disorder, suffer from a disease or disorder, which is known to be associated with a critical extracellular volume status and/or a critical globular volume status, such as edema, brain damage, post-aneurysm rupture, head injury, neurological impairment, multiple traumatic injuries, post-operative, organ failure, disregulated lymphatic flow activity, kidney dysfunction, cardiac dysfunction, and/or disease associated with disordered fluid balance. In particular, the reference subject or the population of reference subjects suffering from a disease or disorder suffer from aneurysm, multiple trauma, brain injury, and/or head injury, or wherein the reference subjects are post-operative subjects suffering from, for example, peritonitis with shock.

If the marker and/or parameter profile from the reference subject contains characteristic features of the marker and/or parameter profile from the at least one reference subject, then the subject to be tested can be diagnosed as respectively being healthy, e.g., having a balanced fluid and or salt balance, or being at risk of developing or having a positive fluid balance and/or salt balance, and/or being at risk or having a critical extracellular volume status and/or a critical globular volume status.

In certain aspects of the invention, the method relates to determining the fluid balance, the salt balance and/or the globular volume status of a subject, wherein the method comprises:
determining a level of proADM or a fragment thereof, preferably MR-proADM, in a sample of said subject, and
comparing said level of proADM or said fragment thereof, preferably MR-proADM, to a reference level of proADM or said fragment thereof, preferably MR-proADM, of at least one reference subject, wherein each reference subject is healthy;
identifying the extracellular volume status, the globular volume status, the fluid balance and/or the salt balance of said subject based on the comparison step; wherein
an increased level of proADM or said fragment thereof, preferably MR-proADM, of the subject as compared to said reference level indicates that said subject has a positive fluid balance, a positive salt balance, a critical globular volume status and/or a critical extracellular volume status;
an identical or similar level of proADM or said fragment thereof, preferably MR-proADM, of the subject as compared to said reference level indicates that said subject has an identical or similar fluid balance, and/or an identical or similar salt balance, wherein said identical fluid balance and/or salt balance indicates that the subject has a normal extracellular volume status and/or a normal globular volume status; and/or
a decreased level of proADM or said fragment thereof, preferably MR-proADM, of the subject as compared to said reference level indicates that said subject has a negative fluid balance and/or a negative salt balance.

In these aspects of the invention, the reference subject is a healthy subject (see above), e.g., a subject having a normal extracellular volume status. The healthy subject has a normal fluid balance and/or salt balance. Healthy subjects normally have a MR-proADM level of about 0.4 to 1 nmol/L (Angeletti S et al., Procalcitonin and mid-regional pro-adrenomedullin test combination in sepsis diagnosis. Clin Chem Lab Med. 2013 May; 51(5):1059-67; Christ-Crain M et al., Mid-regional pro-adrenomedullin as a prognostic marker in sepsis: an observational study. Crit Care. 2005; 9(6):R816-24; or Suzuki Y et al., Development and clinical application of an enzyme immunoassay for the determination of midregional proadrenomedullin. J Pept Sci. 2013 Jan; 19(1):59-63). In one embodiment, the at least one healthy reference subject has a a level of proADM or a fragment thereof, preferably a level of MR-proADM of about 0.5 nmol/L. In another embodiment, the at least one healthy reference subject has a level of proADM or a fragment thereof, preferably a level of MR-proADM of about 0.75 nmol/L. In a further embodiment, the at least one healthy reference subject has a level of proADM or a fragment thereof, preferably MR-proADM of about 1.0 nmol/L. As demonstrated in the appended examples, the subjects suffering, for example, from an aneurysm, multiple trauma or post-surgical disorders showed levels of 1.0 nmol/L or more. In other words, the subjects suffering from a disease or disorder showed high proADM or a fragment thereof, preferably MR-proADM levels. This threshold was also revealed by statistical analysis such as ROC; see FIG. 4 and below. In certain aspects of the invention, when the level of proADM or a fragment thereof, preferably MR-proADM is increased compared to said reference level of healthy subjects, the subject to be tested is considered to have a positive fluid balance, a positive salt balance, a critical globular volume status and/or a critical extracellular volume status. As used herein, an “increased level of proADM or a fragment thereof, preferably MR-proADM of the subject as compared to said reference level” or a “higher” level means that the level of the subject is at least 15%, preferably at least 20%, more preferably at least 25%, or even more preferably at least 30%, higher than the levels of proADM or a fragment thereof, preferably MR-proADM of said healthy reference subjects or of said population of said healthy reference subjects. In certain aspects, the “increased” or “higher” level means that the level of proADM or a fragment thereof, preferably MR-proADM is at least 0.5 nmol/L, for example, at least 0.5 nmol/L, at least 0.75 nmol/L, or at least 1.0 nmol/L.

In certain aspects, the level of proADM or a fragment thereof, preferably MR-proADM is compared to said reference level of healthy subjects, wherein the extracellular volume status, the globular volume status, the fluid balance and/or the salt balance is identified by comparing the level of proADM or a fragment thereof, preferably MR-proADM of the subject to said reference level, wherein an increased level, for example of at least 1 nmol/L, indicates that the subject has positive fluid balance and/or a positive salt balance, and/or wherein said positive fluid balance and/or a positive salt balance indicates that the subjects has a critical globular volume status and/or a critical extracellular volume status.

In certain aspects of the invention, the herein provided method comprises comparing said level of MR-proADM to said reference level of healthy subjects, and wherein an identical or similar level of MR-proADM of the subject as compared to the reference data of healthy subjects indicates that said subject has an identical or similar fluid balance and/or an identical or similar salt balance, wherein said identical or similar fluid balance and/or salt balance indicates that the subject has a normal extracellular volume status and/or a normal globular volume status. As used herein, the “similar level of proADM or said fragment thereof, preferably MR-proADM, of the subject as compared to said reference level” means that the level of proADM or said fragment thereof, preferably MR-proADM, of the subject is +/−10%, preferably, +/−5%, more preferably +/−2% or most preferably the same or identical compared to the levels of proADM or said fragment thereof, preferably MR-proADM, of healthy reference subjects. In an exemplary embodiment, said reference level of proADM or said fragment thereof, preferably MR-proADM, is approximately 0.5 nmol/L to 1.0 nmol/L, wherein the subject has an identical or similar level of proADM or said fragment thereof, preferably MR-proADM, if said level is about 0.5 nmol/L to about 1.0 nmol/L. A normal extracellular volume is, for example, about 15 L of a subject that contains about 40 L of fluid (Guyton Arthur C., (1991), p. 275). As defined herein, a subject with a normal extracellular volume status can have an identical or similar fluid and/or salt balance, thus, the input and output of fluid and/or salt of the subject is in balance, i.e., identical or similar. A normal globular volume status can for example be a globular volume status above 20 ml/kg.

As used herein, the “decreased level of proADM or said fragment thereof, preferably MR-proADM, of the subject as compared to the reference level” means that the level of the subject is 15%, preferably 20%, more preferably 25%, or even more preferably 30%, lower than the reference levels of proADM or said fragment thereof, preferably MR-proADM, of the healthy reference subjects. In preferred aspects, the “decreased” or the “lower” level means that the level of proADM or said fragment thereof, preferably MR-proADM, is below 1.0 nmol/L, for example, below 0.75 nmol/L, or below 0.5 nmol/L. In other words, the subject has a decreased level of proADM or said fragment thereof, preferably MR-proADM, if said level is below 1.0 nmol/L, for example below 0.75 nmol/L, or below about 0.5 nmol/L. In these aspects, a decreased level of proADM or said fragment thereof, preferably MR-proADM, of the subject as compared to the reference data indicates that said subject has a negative fluid balance and/or a negative salt balance.

The sensitivity and specificity of such a method depends on more than just the analytical quality of the test, it also depend on the definition of what constitutes an abnormal or normal result. The distribution of levels of proADM or a fragment thereof, preferably levels of MR-proADM, for subjects with and without a disease/condition might overlap. Under such conditions, a test does not absolutely distinguish normal from disease with 100% accuracy. The skilled person is aware of the fact that the condition per se of a subject or at least one further maker and/or parameter of the subject can assist in the interpretation of the data and that this further information allows a more reliable prognosis in the areas of overlap. Accordingly, the level(s) of at least one further marker and/or parameter is compared to reference data of at least one healthy subject, wherein similar or identical values/levels of said at least one further marker and/or parameter compared to the corresponding levels of said at least one further marker and/or parameter of said reference data indicate that the risk of the subject to have a positive fluid and/or salt balance is decreased, and/or wherein higher or lower levels/values of said at least one further marker and/or parameter compared to the corresponding levels of said at least one further marker and/or parameter of said reference data indicate that the risk to have a positive fluid and/or salt balance is increased and wherein the positive fluid and/or salt balance indicates a critical extracellular volume. In case the reference subject is at least one healthy subject, said similar or identical values/levels of said at least one further marker and/or parameter are normal values/levels, i.e., the values or levels of said markers and parameters are in a normal range. Normal values/levels of makers and parameters are in general known to the skilled person. Normal values/levels of certain markers and parameters are described herein above. In most preferred aspects of the invention, the reference data correspond to or contain the levels of proADM or a fragment thereof, preferably MR-proADM, the body mass index, the weight, the age, the sex, the level of hemoglobin and the level of the total serum protein determined in the reference subjects. The level of proADM or a fragment thereof, preferably MR-proADM, the body mass index, the weight, the age, the sex, the level of hemoglobin and the level of the total serum protein of the subject to be tested are compared to the reference data of such reference subjects.

As used herein, “similar or identical” level/value means that the level/value is +/−10%, preferably, +/−5%, more preferably +/−2% or most preferably the same or identical compared to the corresponding level/value. As used herein, “lower” or “decreased” or “higher” or “increased” level/value means that the level/value is 15%, preferably 20%, more preferably 25%, or most preferably 30%, higher or lower, respectively, compared to the corresponding level/value.

In an exemplary embodiment of the invention, the method relates to determining the fluid balance, the salt balance and/or the globular volume status of a subject, wherein the method comprises:
determining the level of proADM or a fragment thereof, preferably MR-proADM, in a sample of said subject, and
comparing said level of proADM or said fragment thereof, preferably MR-proADM, to a reference level of proADM or said fragment thereof, preferably MR-proADM, of at least one reference subject or a population of reference subjects, wherein the reference subjects are subjects suffering from a disease or disorder which is known to be associated with a critical extracellular volume status and/or a critical globular volume status or wherein the reference subjects are post-operative subjects suffering from peritonitis with shock; and
identifying the extracellular volume status, the globular volume status, the fluid balance and/or the salt balance of said subject based on the comparison step; wherein
a similar level, identical level or increased level of proADM or said fragment thereof, preferably MR-proADM, of the subject as compared to said reference level indicates that said subject has a positive fluid balance, a positive salt balance, a critical globular volume status and/or a critical extracellular volume status; and/or
a decreased level of proADM or said fragment thereof, preferably MR-proADM, of the subject as compared to said reference data indicates that said subject has a normal fluid balance, normal salt balance, normal extracellular volume status and/or normal globular volume status.

In certain preferred aspects of the invention, the reference subjects are subjects suffering from a disease or disorder which is known to be associated with a critical extracellular volume status and/or a critical globular volume status. Such disease or disorders include for instance aneurysm, multiple trauma, brain injury, and/or head injury, or cases wherein the reference subjects are post-operative subjects suffering from peritonitis with shock. Therefore, it is envisaged that the disease or disorder involves conditions, wherein the fluid balance, the salt balance, the body fluid, the extracellular volume and/or the intracellular volume is/are critical. Therefore, in an exemplary embodiment, the reference subject suffers from aneurysm, multiple trauma, brain injury and/or head injury, and/or is a post-operative subject suffering from a disease or disorder, such as peritonitis with shock. In another embodiment, the reference subject is a subject suffering from a disease or disorder selected from aneurysm (ANE), traumatic brain injury (TC), multiple trauma (POLY), digestive surgery (CD), severe brain trauma (SBT), aneurysmal subarachnoid haemorrhage (SAH), severe trauma without head trauma (PT) and post-surgical peritonitis with shock (P). The level of MR-proADM of the subject to be tested is compared to the reference data of such reference subjects. It is shown in the appended examples that the threshold for proADM or a fragment thereof, preferably MR-proADM predicting critical extracellular volume states was identified by plotting proADM or a fragment thereof, preferably MR-proADM ROC curves of subjects having a disease or disorder for predicting the fluid balance and salt balance; see Example 1 and FIG. 4. It is shown therein that a high level of MR-proADM, e.g., at least about 1.0 to at least about 1.5 nmol/L, indicates a gain of water/fluid and/or sodium/salt. Thus, in preferred aspects, said level of proADM or a fragment thereof, preferably MR-proADM determined is compared to the reference level of reference subjects suffering from a disease or disorder, which is known to be associated with a critical extracellular volume status, such as aneurysm, multiple trauma, brain injury, and/or head injury, or wherein the reference subjects are post-operative subjects suffering from peritonitis with shock, wherein a high or increased level, for example of at least 1 nmol/1 determines that the subject has a positive fluid balance and/or salt balance. In other words, a similar or identical level or an even increased level of proADM or a fragment thereof, preferably MR-proADM determined compared to reference level of reference subjects suffering from said disease or disorder indicates that said subject has a positive fluid balance, a positive salt balance, a critical globular volume status and/or a critical extracellular volume status. As used herein, the “similar level or identical level of proADM or said fragment thereof, preferably MR-proADM, of the subject as compared to said reference level” means that the level of proADM or said fragment thereof, preferably MR-proADM of the subject is +/−10%, preferably +/−5%, more preferably +/−2% or even more preferably the same or identical compared to the level of proADM or said fragment thereof, preferably MR-proADM, of at least one a reference subject suffering from said disease and/or disorder. As used herein, the “increased level of proADM or said fragment thereof, preferably MR-proADM, of the subject as compared to said reference level” means that the level of the subject is at least 15%, preferably at least 20%, more preferably at least 25%, or even more preferably at least 30%, higher than the levels of proADM or said fragment thereof, preferably MR-proADM, of said reference subjects suffering from said disease or disorder. In other words, the “similar level or identical level or increased level of proADM or said fragment thereof, preferably MR-proADM, of the subject as compared to said reference level” means that the level of proADM or said fragment thereof, preferably MR-proADM, of the subject is +/−10%, preferably +/−5%, more preferably +/−2% or even more preferably the same or identical compared to the level of proADM or said fragment thereof, preferably MR-proADM, of at least a reference subject suffering from said disease and/or disorder; or is at least 15%, preferably at least 20%, more preferably at least 25%, or even more preferably at least 30%, higher than the levels of proADM or said fragment thereof, preferably MR-proADM, of said reference subjects suffering from said disease or disorder. In one aspect, when the reference subjects are subjects suffering from said disease or disorder, the “similar level or identical level or increased level of proADM or said fragment thereof, preferably MR-proADM, of the subject” means that the level of proADM or said fragment thereof, preferably MR-proADM, is about 0.5 nmol/L or at least 0.5 nmol/L. In another aspect, the “similar or identical level or increased level of proADM or said fragment thereof, preferably MR-proADM, of the subject” means that the level of proADM or said fragment thereof, preferably MR-proADM, is about 0.75 nmol/L or at least 0.75 nmol/L. In a further aspect, the “similar or identical level or increased level of proADM or said fragment thereof, preferably MR-proADM, of the subject” means that the level of proADM or said fragment thereof, preferably MR-proADM, is about 1 nmol/L or at least 1 nmol/L or even about 1.5 nmol/L or at least 1.5 nmol/L. In other words, said reference level of proADM or said fragment thereof, preferably MR-proADM, is in the range of 0.5 nmol/L to 1.5 nmol/L, for example 0.5 nmol/L, 0.75 nmol/L, or 1.0 nmol/L, wherein the subject has a similar or identical level or increased level of proADM or said fragment thereof, preferably MR-proADM, if said level is about or at least 0.5 nmol/L to about or at least 1.5 nmol/L.

When the level of proADM or a fragment thereof, preferably MR-proADM determined in the subject is decreased or lower compared to subjects having said disease and/or a disorder, the subject to be tested does not have a positive fluid and/or salt balance, but rather have a normal fluid and/or salt balance, i.e., an identical or similar fluid balance and/or salt balance. Therefore, the fluid and/or salt balance is in balance. Therefore, this subject has a normal fluid balance, normal salt balance, normal extracellular volume status and/or normal globular volume status. This decreased or lower level may also indicate that the subject has a negative salt balance and/or fluid a balance. As used herein, the term “decreased level of proADM or said fragment thereof, preferably MR-proADM, of the subject” means that the level of proADM or said fragment thereof, preferably MR-proADM, determined in the subject to be tested has at least 15%, preferably at least 20%, more preferably at least 25%, or even more preferably at least 30%, lower level of proADM or said fragment thereof, preferably MR-proADM, compared to the levels of the reference subjects suffering from said disease or disorder. In preferred aspects, the “decreased level of proADM or said fragment thereof, preferably MR-proADM, of the subject” or “lower” level means that the level of proADM or said fragment thereof, preferably MR-proADM, is below 1.0 nmol/L, for example, below 1.0 nmol/L, below 0.75 nmol/L, or below 0.5 nmol/L. In other words, said reference level of proADM or said fragment thereof, preferably MR-proADM, is in the range of 0.5 nmol/L to 1.0 nmol/L, wherein the subject has a decreased level of proADM or said fragment thereof, preferably MR-proADM, if said level is below 1.0 nmol/L, for example, below 1.0 nmol/L, below 0.75 nmol/L, or below 0.5 nmol/L.

In the area of overlap, the determination of further conditions of the subject can assist in the prognosis. Accordingly, the level(s) of at least one further marker and/or parameter is compared to reference data of at least one subject suffering from a disease or disorder, wherein similar or identical of said at least one further marker and/or parameter values/levels increase the risk to have a positive fluid and/or salt balance, and wherein higher or lower levels/values of said at least one further marker and/or parameter decrease the risk of the subject to have a positive fluid and/or salt balance, and wherein the positive fluid and/or salt balance indicate a critical extracellular volume.

As shown in the appended examples, the combination of markers and parameters are selected to yield the lowest error. This selection or importance analysis is done with standard statistical analysis, e.g., random forest analysis. As was shown in the appended examples, the markers and parameters proADM or a fragment thereof, preferably MR-proADM, body mass index, weight, age, sex, hemoglobin and total serum protein of the subject yield a very reliable prediction of critical patients that are suffering from a positive fluid and/or salt balance. Therefore, in preferred aspects of the invention, the prediction of patient outcome, i.e., the fluid balance and/or salt balance of the subject, is performed with standard statistical analysis, such as random forest. In these aspects, the markers and parameters are implemented in a formula, which can be integrated in a software program. Therefore, in certain embodiments, the invention relates to software suitable for determining the fluid balance, the salt balance, the extracellular volume and/or the globular volume of the subject employing the method provided herein. Accordingly, the level of proADM or a fragment thereof, preferably MR-proADM is determined in the sample of the subject and entered in the software. In other embodiments, the level of proADM or a fragment thereof, preferably MR-proADM, the body mass index, the weight, the age, the sex, the level of hemoglobin and the level of the total serum protein of the subject is determined and entered in the software. In further embodiments, the software automatically calculates/determines the p-critical based on the levels of proADM or a fragment thereof, preferably MR-proADM and/or further parameters and markers and determines whether a subject has a critical fluid balance, critical salt balance, critical extracellular volume and/or critical globular volume. In other words, the software gives a prognosis whether the subject is a critical subject or not. Such software can be employed by a graphical user interface. The formula behind the interface is generated automatically using standard statistical methods, e.g., Random Forest, implemented in the open scientific software R and based on patient data. The statistical analysis thus compares the levels of the markers and parameters to the reference data and predicts the fluid balance and/or the salt balance and thus the extracellular volume status of the subject. In the ICU, the physician may use the interface to enter the markers and/or parameters to obtain an estimate of the fluid balance and salt balance, which might be used to identify subjects with a positive fluid balance and/or salt balance. In case the fluid balance or salt balance is more than 41 for the fluid balance and more than 36 g for salt balance, the patient is in critical phase (p-critical >60%). The results of the prediction can be illustrated in the graphical user interface, e.g., by a traffic light system. For example, values of fluid balance and salt balance that are more than 4 L or 36 g; respectively, are highlighted in red as they indicate a critical patient. If the patient has a fluid balance or salt balance below 4 L or 36 g, respectively, the patient or the values are highlighted in green (patients have a p-critical below 30%). In these aspects, the method herein provided can be employed: for treatment guiding, for example, if p-critical is more than 60%, fluid management is reconsidered; for diagnosis of positive fluid and salt balance, to inform the clinician that this patient has a fluid overload, even for patients not receiving intravenous fluid resuscitation; or prognosis patient in case the patient has a p-critical >90%, the patient has a high SOFA score, low RBCV and thus an even poorer prognosis (FIG. 6). Between 30% and 40%, the patient has an intermediate p-critical that is highlighted in yellow.

In certain aspects of the invention, the method relates to determining the fluid balance, the salt balance and/or the globular volume status of a subject, wherein the method comprises:
determining in a sample obtained from said subject the level of the marker proADM or a fragment thereof, preferably the level of MR-proADM;
comparing said level of proADM or said fragment thereof, preferably the level of MR-proADM, to (a) level(s) of proADM or said fragment thereof, preferably the level(s) MR-proADM, of the same subject obtained from prior analysis; and
identifying the extracellular volume status, the globular volume status, the fluid balance and/or the salt balance of said subject based on the comparison step, wherein a level of at least 1 nmol/L indicates that the subject has a positive fluid balance, a positive salt balance, a critical globular volume status and/or critical extracellular volume status.

In certain aspects of the invention, the term “comparing said level of proADM or said fragment thereof, preferably the level of MR-proADM, to (a) level(s) of proADM or said fragment thereof, preferably the level(s) MR-proADM, of the same subject obtained from prior analysis” means that the level of proADM or said fragment thereof, preferably the level of MR-proADM, is determined as described herein and that this level of proADM or said fragment thereof, preferably MR-proADM, is compared to the level of proADM or said fragment thereof, preferably MR-proADM, or the levels of proADM or said fragment thereof, preferably MR-proADM, that is/are obtained from the same subject at a prior analysis. Preferably, the level of proADM or said fragment thereof, preferably MR-proADM, is determined at several time points, i.e., more than one level of proADM or said fragment thereof, preferably MR-proADM, is available obtained from prior analysis. A series can be calculated with these levels determined at different time points. This series shows a trend, which can be employed to determine e.g. the extracellular volume status and/or the globular volume status of the subject. In other words, the trend of the level of MR-proADM predicts the extracellular volume state. For example, in case a series of measurements of levels/values, e.g., of MR-proADM, has been determined at several prior time points, the skilled person can calculate a trend which can be used interpret the development of proADM or a fragment thereof, preferably MR-proADM and/or the further markers and/or parameters. For example, a positive trend, i.e., the values increase or are higher than the levels measured before, can predict that the subject has a positive fluid balance and/or salt balance. In certain aspects, if the levels of proADM or a fragment thereof, preferably MR-proADM, obtained from prior analysis of the same subject show a positive trend and at least one level of proADM or a fragment thereof, preferably MR-proADM, obtained from prior analysis of the same subject is in the range of at least 0.5 nmol/L to at least 1.5 nmol/L, for example, at least 0.5 nmol/L, at least 0.75 nmol/L, at least 1 nmol/L or at least 1.5 nmol/L, the subject is indicated to have a positive fluid balance and/or positive salt balance, wherein a positive fluid balance and/or salt balance indicate that the subject has critical extracellular volume status and/or a critical globular volume status, wherein the health status of the subject deteriorates. Similarly, in certain aspects, if the levels of proADM or a fragment thereof, preferably MR-proADM, levels obtained from prior analysis of the same subject show a negative trend and at least one level of proADM, or a fragment thereof, preferably MR-proADM, obtained from prior analysis of the same subject is, for example, at least 0.5 nmol/L, at least 0.75 nmol/L, or at least 1 nmol/L, the subject is indicated to have a positive fluid balance and/or a positive salt balance, wherein a positive fluid balance and/or salt balance indicate that the subject has or had a critical extracellular volume status or a critical globular volume status, wherein the health status alleviates. In the appended examples, the level of proADM or a fragment thereof, preferably MR-proADM, decreases with the time of treatment. Without being bound by theory, the decrease of the proADM or a fragment thereof, preferably MR-proADM, concentration might be due to the alleviated endothelial damage.

In certain aspects, certain fixed thresholds are employed to determine the extracellular volume state of the subject. In one embodiment, when the level of proADM or a fragment thereof, preferably MR-proADM, is higher than 0.5 nmol/1, the patient is determined to have a positive fluid balance and/or salt balance, wherein the positive fluid balance and/or salt balance indicates that the subject has a critical extracellular volume. In another embodiment, when the level of proADM or a fragment thereof, preferably MR-proADM, is higher than 0.75 nmol/1, the patient is determined to have a positive fluid balance and/or salt balance, wherein the positive fluid balance and/or salt balance indicates that the subject has a critical extracellular volume. In another embodiment, when the level of proADM or a fragment thereof, preferably MR-proADM, is higher than 1.0 nmol/1, the patient is determined to have a positive fluid balance and/or salt balance, wherein the positive fluid balance and/or salt balance indicates that the subject has a critical extracellular volume. In another embodiment, when the level of proADM or a fragment thereof, preferably MR-proADM, is higher than 1.5 nmol/1, the patient is determined to have a positive fluid balance and/or salt balance, wherein the positive fluid balance and/or salt balance indicates that the subject has a critical extracellular volume.

In certain aspects of the invention, the term “comparing said level of proADM or said fragment thereof, preferably MR-proADM, and said level(s) of at least one further marker and/or parameter to data corresponding to said level of proADM or said fragment thereof, preferably MR-proADM, and said level(s) of at least one further marker and/or parameter of the same subject obtained from prior analysis” means that the level of proADM or said fragment thereof, preferably MR-proADM, is determined and at least one further level of at least one further marker and/or at least one further parameter is determined and that the level of proADM or said fragment thereof, preferably MR-proADM, is compared to a corresponding level of proADM or said fragment thereof, preferably MR-proADM, of the same subject that is determined at an earlier analysis and that the level(s) of the at least one further marker and/or at least one further parameter is compared to the corresponding level(s) of the at least one further marker and/or at least one further parameter of the same subject that is determined at an earlier analysis. The levels of the at least one further marker and/or parameter obtained from prior analysis can be compared to itself in order to predict a trend based on the multivariates. Alternatively, the further markers and/or parameters can be compared to normal data, e.g., data of healthy reference subjects. In case the further markers and/or parameters are higher or lower than the normal levels, the risk of a positive fluid balance and/or salt balance is increased, i.e., the subject is more susceptible to a critical extracellular volume status.

In certain aspects of the invention, the level of proADM or a fragment thereof, preferably MR-proADM, the body mass index, the weight, the level of hemoglobin and the level of the total serum protein are compared to a corresponding level of the same subject that are determined at an earlier analysis. In other words, the levels of the markers and parameters are determined at different time points and the trends of the levels predict the extracellular volume state.

As used herein, “prior analysis” means that the level of the marker is determined at several time points during the hospitalization, e.g., day 0, day 1, day 2, day 3, day 4, day 5, day 6, day 7, etc. The determination of the markers and/or parameters can also be performed hourly, e.g., a first measurement can be performed at admission of the patient and then the measurement can be repeated, for example, every hour, every two hours or every five hours. The level of the maker, e.g., proADM or a fragment thereof, preferably MR-proADM, and optionally the level of the parameter determined are compared to either one level/value of the marker or parameter determined at an earlier time point or an average of levels/values calculated from two or more earlier time points. The change of the level of the marker indicates the extracellular volume status of the subject.

As shown in the appended Example 1 and FIG. 4, thresholds of MR-proADM were identified for predicting critical patients, e.g., at least 1 nmol/1, by plotting MR-proADM ROC curves for predicting the fluid balance and salt balance of intensive care patients; e.g., patients suffering from aneurysmal subarachnoid haemorrhage (SAH), severe trauma without head trauma (PT), severe brain trauma (SBT) or post-surgical peritonitis with shock patients (P). It is demonstrated herein that a high or increased level of proADM or a fragment thereof, preferably MR-proADM, e.g., above than 1 nmol/1, indicates that the subject has a fluid overload, i.e., a positive fluid balance. Thus, in preferred aspects of the present invention, the herein provided method comprises determining the level of proADM or a fragment thereof, preferably MR-proADM in a subject, wherein an increased level of proADM or a fragment thereof, preferably MR-proADM indicates that said subject has a positive fluid balance and/or a positive salt balance.

It is shown in the appended examples that a high level of proADM or a fragment thereof, preferably MR-proADM, e.g., at least about 1.0 to at least about 1.5 nmol/L, indicates a gain of water/fluid and/or sodium, i.e., a positive fluid balance and/or positive salt balance of the subject, for example, of at least about 3 L to 4 L or about 27 g to 36 g sodium/salt, respectively. Therefore, in embodiments of the invention, an increased or high level of proADM or a fragment thereof, preferably MR-proADM of the subject is at least 0.5 nmol/L, or at least 0.6 nmol/L, or at least 0.7 nmol/L, or at least 0.75 nmol/L, or at least 0.8 nmol/L, or at least 0.9 nmol/L, or at least 1.1 nmol/L, or at least 1.2 nmol/L, or at least 1.3 nmol/L, or at least 1.4 nmol/L, or at least 1.5 nmol/L, or at least 1.0 nmol/L.

In the appended examples, different patient groups, i.e., patients suffering from aneurysmal subarachnoid haemorrhage (SAH), severe trauma without head trauma (PT), severe brain trauma (SBT) or post-surgical peritonitis with shock patients (P) showed all increased or high MR-proADM levels. In particular, patients suffering from post-surgical peritonitis with shock patients (P) demonstrated especially high values of MR-proADM at day 0, day 2 and day 7; see FIG. 3. Therefore, it is envisaged herein that the level of proADM or a fragment thereof, preferably MR-proADM can vary dependent on the patient group and certain disorders such as post-surgical can result in even higher levels of proADM or a fragment thereof, preferably MR-proADM that are suitable to identify a critical volume status, positive fluid and/or salt balance. Thus, it is envisaged herein that plotting of proADM or a fragment thereof, preferably MR-proADM levels in ROC (see below) for predicting the fluid balance and salt balance of patients suffering from a specific disease can result in higher or lower thresholds than 1 nmol/L. For example, a level of proADM or a fragment thereof, preferably MR-proADM of at least 1.5 nmol/L in a post-operative subject indicates a positive fluid balance and/or positive salt balance. In general, an increased value of at least 1.0 nmol/L indicates a subject with a positive fluid and/or salt balance. In preferred aspects of the invention, a level of proADM or a fragment thereof, preferably MR-proADM determined in a subject is considered as increased, if the concentration of proADM or a fragment thereof, preferably MR-proADM is at least 1 nmol/L (concentration [MR-proADM] >1.0 nmol/L). In other words, a concentration of more than 1 nmol/L of proADM or a fragment thereof, preferably MR-proADM in a subject indicates a positive fluid balance (for example, of at least 4 L) or a gain of water. Alternatively, a concentration of more than 1.0 nmol/L of proADM or a fragment thereof, preferably MR-proADM in a subject indicates a positive salt balance (for example, of at least 36 g) or a gain of salt or a critical extracellular volume status.

In preferred aspects of the invention, the method provided herein determines the extracellular volume of a subject, wherein the method comprises determining in a sample obtained from said subject the level of the marker proADM or a fragment thereof, preferably MR-proADM, wherein based on the level of proADM or a fragment thereof, preferably MR-proADM the fluid balance is determined and wherein said fluid balance determines the extracellular volume status, wherein an increased level of proADM or a fragment thereof, preferably MR-proADM of the subject indicates that said subject has a positive fluid balance, wherein the increased level of MR-proADM is at least 1 nmol/L, wherein said level indicates that said positive fluid balance is at least about 4 L, and wherein said positive fluid balance indicates that said subject has an extracellular volume state that is considered as critical.

In preferred aspects, the method provided herein determines the extracellular volume of a subject, wherein the method comprises determining in a sample obtained from said subject the level of the marker proADM or a fragment thereof, preferably MR-proADM, wherein based on the level of proADM or a fragment thereof, preferably MR-proADM the salt balance is determined and wherein said salt balance determines the extracellular volume status, wherein an increased level of proADM or a fragment thereof, preferably MR-proADM of the subject indicates that said subject has a positive salt balance, wherein the increased level of proADM or a fragment thereof, preferably MR-proADM is at least 1 nmol/L, wherein said level indicates that said positive salt balance is at least about 36 g, and wherein said positive salt balance indicates that said subject has an extracellular volume state that is considered as critical.

As used herein, a “sample” in the meaning of the invention can be any fluid of the subject such as plasma, lymph, urine, cerebral fluid, blood, saliva, serum, or faeces and any biological tissue of the subject.

Preferably, the sample is a blood sample, more preferably a serum sample or, most preferably a plasma sample in the context of the present invention.

In preferred aspects of the present invention, the level of proADM or a fragment thereof, preferably MR-proADM is determined in the sample, wherein said sample is a blood or plasma sample. In most preferred aspects, the maker is determined in a plasma sample.

It is envisaged herein that the sample may be a tissue, e.g., pulmonary tissue, ascites, skin, heart, kidney, digestive tract, or lower lim oedema, epithelium tissue, connective tissue such as bone or blood, muscle tissue such as visceral or smooth muscle and skeletal muscle, nervous tissue, bone marrow, cartilage, skin, mucosa or hair. The sample is collected/obtained from the patient or subjected to the diagnosis according to the invention. Where appropriate, as for instance in the case of solid samples, the sample may need to be solubilized, homogenized, or extracted with a solvent prior to use in the present invention in order to obtain a liquid sample. In preferred aspects, the sample is a liquid sample, e.g., a solution or suspension. Liquid samples may be subjected to one or more pre-treatments prior to use in the present invention. Such pre-treatments include, but are not limited to dilution, filtration, centrifugation, concentration, sedimentation, precipitation, or dialysis. Pre-treatments may also include the addition of chemical or biochemical substances to the solution, such as acids, bases, buffers, salts, solvents, reactive dyes, detergents, emulsifiers, or chelators. In preferred aspects, said sample is blood, blood plasma, blood serum or urine. In most preferred aspects, the sample is blood plasma.

“Plasma” in the context of the present invention is the virtually cell-free supernatant of blood containing anticoagulant obtained after centrifugation. Exemplary anticoagulants include calcium ion binding compounds such as EDTA or citrate and thrombin inhibitors such as heparinates or hirudin. Cell-free plasma can be obtained by centrifugation of the anticoagulated blood (e.g. citrated, EDTA or heparinized blood), for example for at least 15 minutes at 2000 to 3000 g. “Serum” in the context of the present invention is the liquid fraction of whole blood that is collected after the blood is allowed to clot. When coagulated blood (clotted blood) is centrifuged serum can be obtained as supernatant.

The level of proADM or a fragment thereof, preferably MR-proADM and/or the level of further markers can be determined by an immunoassay. As used herein, an “assay” or a diagnostic assay can be of any type applied in the field of diagnostics. Preferred detection methods comprise immunoassays in various formats such as for instance radioimmunoassays, chemiluminescence-and fluorescence-immunoassays, Enzyme-linked immunoassays (ELISA), Luminex-based bead arrays, protein microarray assays, assays suitable for point-of-care testing and rapid test formats such as for instance immune-chromatographic strip tests. Such an assay may be based on the binding of an analyte to be detected to one or more capture probes with a certain affinity. As used herein, an immunoassay is a biochemical test that measures the presence or concentration of a macromolecule/polypeptide in a solution through the use of an antibody or immunoglobulin. According to the invention, the antibodies may be monoclonal as well as polyclonal antibodies.

Thus, at least one antibody is a monoclonal or polyclonal antibody. The method according to the present invention is particularly preferred, wherein the midregional partial peptide spanning amino acids 42-95 of pre-proADM or amino acids as given in SEQ ID NO: 2 is employed for the determination of MR-proADM or partial peptides thereof in a sample. In certain aspects, the level of the marker is determined by high performance liquid chromatography (HPLC). In certain aspects, the HPLC can be coupled to an immunoassay.

In certain aspects of the present invention, proADM or a fragment thereof, preferably MR-proADM or a fragment thereof and/or further markers or fragments thereof are determined with a sandwich immunoassay. In this sandwich immunoassay, two antibodies are applied for, e.g., one marker such as proADM or a fragment thereof, preferably MR-proADM, in a sample. In particular, this is preferred, if proADM or a fragment thereof, preferably MR-proADM or a fragment thereof are determined by the use of two antibodies, which specifically bind to different partial sequences of proADM or a fragment thereof, preferably MR-proADM or a fragment thereof.

In a preferred aspect of the in vitro method according the invention, one of the antibodies is labeled and the second one is bound to or may be bound selectively to a solid phase. In a particularly preferred aspect of the assay, one of the antibodies is labeled while the other is either bound to a solid phase or can be bound selectively to a solid phase. In a preferred embodiment the method is executed as heterogeneous sandwich immunoassay, wherein one of the antibodies is immobilized on an arbitrarily chosen solid phase, for example, the walls of coated test tubes (e.g. polystyrol test tubes; coated tubes; CT) or microtiter plates, for example composed of polystyrol, or to particles, such as for instance magnetic particles, whereby the other antibody has a group resembling a detectable label or enabling for selective attachment to a label, and which serves the detection of the formed sandwich structures. A temporarily delayed or subsequent immobilization using suitable solid phases is also possible.

The method according to the present invention can furthermore be embodied as a homogeneous method, wherein the sandwich complexes formed by the antibody/antibodies and the marker, e.g., proADM or a fragment thereof, preferably MR-proADM or a fragment thereof, which is to be detected remains suspended in the liquid phase. In this case it is preferred, that when two antibodies are used, both antibodies are labeled with parts of a detection system, which leads to generation of a signal or triggering of a signal if both antibodies are integrated into a single sandwich. Such techniques are to be embodied in particular as fluorescence enhancing or fluorescence quenching detection methods. A particularly preferred aspect relates to the use of detection reagents which are to be used pair-wise, such as for example the ones which are described in U.S. Pat. No. 4,882,733A, EP-B1 0 180 492 or EP-B1 0 539 477 and the prior art cited therein. In this way, measurements in which only reaction products comprising both labeling components in a single immune-complex directly in the reaction mixture are detected, become possible. For example, such technologies are offered under the brand names TRACE® (Time Resolved Amplified Cryptate Emission) or KRYPTOR®, implementing the teachings of the above-cited applications. Therefore, in particular preferred aspects, a diagnostic device is used to carry out the herein provided method. For example, the level of proADM or a fragment thereof, preferably MR-proADM and/or the level of any further marker of the herein provided method is determined. In particular preferred aspects, the diagnostic device is KRYPTOR®.

The invention further relates to the use of a kit for determining the extracellular volume status in a sample obtained from a test subject comprising detection reagents for determining at least one marker selected from the group consisting of proADM or a fragment thereof, preferably MR-proADM, hemoglobin, total serum protein, renin, pro-atrial natriuretic peptide (proANP), C-terminal pro-arginine-vasopressin (CT-proAVP), protein, erythropoietin, angiotensin II, aldosterone, cortisol, adrenaline, epinephrine, catecholamines and pro-endothelin-1 (pro-ET-1) or a fragment thereof, and comprising ancillary substances for carrying out the herein provided method. In certain aspects, the invention relates to the use of a kit for determining the extracellular volume status in a sample obtained from a test subject comprising detection reagents for determining the level of proADM or a fragment thereof, preferably MR-proADM or the fragment thereof, and comprising ancillary substances for carrying out the herein provided method. In preferred aspects, the invention relates to the use of a kit for determining the extracellular volume status in a sample obtained from a test subject comprising detection reagents for determining the markers proADM or a fragment thereof, preferably MR-proADM, hemoglobin and total serum protein and comprising ancillary substances for carrying out the herein provided method.

In certain aspects, said detection reagents for determining the level of proADM or a fragment thereof, preferably MR-proADM or the fragment thereof comprise antibodies, wherein one of the antibodies is labelled and the other antibody is bound to a solid phase or can be bound selectively to a solid phase.

In certain aspects, said detection reagents for determining the level of at least one marker comprise antibodies, wherein one of the antibodies is labelled and the other antibody is bound to a solid phase or can be bound selectively to a solid phase.

In principle, all labeling techniques which can be applied in assays of said type can be used, such as labeling with radioisotopes, enzymes, fluorescence-, chemoluminescence- or bioluminescence labels and directly optically detectable color labels, such as gold atoms and dye particles, which are used in particular in Point-of-Care (POC) or rapid tests. In the case of heterogeneous sandwich immunoassays, both antibodies may exhibit parts of the detection system according to the type described herein in the context of homogenous assays.

In a preferred aspect, both the first and the second antibody are dispersed in the liquid reaction medium, whereby a first labeling component which is part of a labeling system based on fluorescence- or chemoluminescence quenching or enhancement is bound to the first antibody, and whereby the second labeling component of this labeling system is bound to the second antibody, such that after binding of both antibodies to the marker, e.g., proADM or a fragment thereof, preferably MR-proADM or the fragment thereof or the further marker or the fragment thereof, which is to be detected, a detectable signal is generated which enables for a detection of the sandwich complexes formed in the measuring solution. One aspect of this alternative comprises the labeling system such as rare earth kryptates or chelates in combination with a fluorescence- or cheminoluminescence-dye. In a particular preferred aspect, the labeling system comprises a rare earth kryptate in combination with a fluorescence or chemiluminescence dye, in particular, of the cyanine type. In a further preferred aspect, the detection is carried out with a competitive immunoassay. In a preferred aspect, a radioimmunoassay is used. It also envisaged herein that the level of the marker can be, for example, determined by mass spectrometric methods or by a high performance liquid chromatography (HPLC) method, which can be coupled to an immunoassay, or a mass-spectrometric based approach. The skilled person understands that any available assay can be used as long as the level of the marker can be reliably determined.

An object of the invention is to provide an in vitro method for diagnosis, prognosis, risk assessment, risk stratification, therapy control and/or operative control of a disorder or medical condition in a subject and/or a patient, which provides reliable information especially to the medical practitioner in the Emergency Department (ED) or Intensive Care Unit (ICU).

Thus, the invention relates to the method for in vitro diagnosis, prognosis, risk assessment, risk stratification, therapy control and/or operative control of a disorder or medical condition in a subject, wherein the extracellular volume status, the globular volume status, the fluid balance and/or the salt balance of said subject is determined by the herein provided method.

In one embodiment, the invention relates to the method for in vitro diagnosis, prognosis, risk assessment, risk stratification, therapy control and/or operative control of a disorder or medical condition in a subject, wherein based on the level of proADM or a fragment thereof, preferably MR-proADM the fluid balance, the salt balance and/or the globular volume status of the subject is determined.

In other embodiments, the invention relates to the method for in vitro diagnosis, prognosis, risk assessment, risk stratification, therapy control and/or operative control of a disorder or medical condition in a subject, wherein based on the level of proADM or a fragment thereof, preferably MR-proADM the fluid balance and/or the salt balance is determined and wherein said fluid balance and/or the salt balance determines the extracellular volume status.

In further embodiments, the invention relates to the method for in vitro diagnosis, prognosis, risk assessment, risk stratification, therapy control and/or operative control of a disorder or medical condition in a subject, wherein based on the level of proADM or a fragment thereof, preferably MR-proADM, the level of hemoglobin, the level of the total serum protein, the weight of the subject, the age of the subject and the sex of the subject the fluid balance, the salt balance and/or the globular volume status is determined.

As used herein, “diagnosis” in the context of the present invention relates to the recognition and (early) detection of a disease or clinical condition in a subject and may also comprise differential diagnosis. Also the assessment of the severity of a disease or clinical condition may in certain embodiments be encompassed by the term “diagnosis”.

As used herein, “prognosis” relates to the prediction of an outcome or a specific risk for a subject suffering from a particular disease or clinical condition. This may include an estimation of the chance of recovery or the chance of an adverse outcome for said subject.

The term “therapy control” in the context of the present invention refers to the monitoring and/or adjustment of a therapeutic treatment of said patient. “Monitoring” relates to keeping track of an already diagnosed disease, disorder, complication or risk, e.g. to analyze the progression of the disease or the influence of a particular treatment on the progression of disease or disorder.

In the present invention, the terms “risk assessment” and “risk stratification” relate to the grouping of subjects into different risk groups according to their further prognosis. Risk assessment also relates to stratification for applying preventive and/or therapeutic measures. As used herein, the term “operative control” relates to the pre-operative control intra-operative control and/or to the post-operative control of a subject. In particular, it means herein that the fluid balance, the salt balance, the globular volume status and/or the extracellular volume status is controlled. Therefore, the fluid and/or the salt is monitored and controlled in such subjects.

In certain aspects, the disorder or medical condition can be water overload, edema, brain damage, post-aneurysm rupture, severe head injury, neurological impairment, severe multiple traumatic injuries, post-operative, cardiac risk, kidney injury, organ failure, disregulated lymphatic flow activity, kidney dysfunction, cardiac dysfunction, disease associated with disordered fluid balance.

As shown in the appended examples, a significant statistical relationship between MR-proADM and the fluid and/or salt balance of a subject was found. The fluid and/or salt balance is indicative for the extracellular volume of subject and/or patient. As was demonstrated in the appended examples, this strong relationship was found in several clinical situations of the patients, such as patients with severe brain trauma (SBT), aneurismal subarachnoid haemorrhage (SAH), severe trauma without head trauma (PT) and post-surgical peritonitis with shock (P) (e.g., Example 1). Therefore, in certain aspects, the invention relates to a method for in vitro diagnosis, prognosis, risk assessment, risk stratification, therapy control and/or operative control of a disorder or medical condition in a subject, wherein said subject has a brain or head injury, multiple traumatic injuries, or an aneurysm or is post-operative. In further aspects, the invention relates to a method for in vitro diagnosis, prognosis, risk assessment, risk stratification, therapy control and/or operative control of a disorder or medical condition in a subject, wherein said subject has severe brain trauma (SBT), aneurismal subarachnoid haemorrhage (SAH), severe trauma without head trauma (PT) and post-surgical peritonitis with shock (P). In further aspects, the invention relates to a method for in vitro diagnosis, prognosis, risk assessment, risk stratification, therapy control and/or operative control of a disorder or medical condition in a subject, wherein said subject has a post-aneurysm rupture or severe head injury. In certain aspects, said subject has no neurological impairment. In certain aspects, said subject has severe multiple traumatic injuries or is post-operative.

The herein provided method can be employed in the fluid management of the subject or a patient. As used herein, the term “fluid management” means the monitoring and controlling of the fluid status of a subject or a patient and the administration of fluid, e.g., by intravenous fluid administration. Thus, in certain aspects, the invention relates to a method for use in the fluid management of a subject, wherein said extracellular volume status of said subject is determined by the herein provided method. In certain aspects, the invention relates to a method for use in the fluid management of a subject, wherein based on the level of proADM or a fragment thereof, preferably MR-proADM the extracellular volume status of the subject is determined by the herein provided method.

In certain aspects, the invention relates to the herein provided method for use in the fluid management of a subject, wherein based on the level of proADM or a fragment thereof, preferably MR-proADM, and/or wherein based on the fluid and/or the salt balance of the subject the therapy of the disorder or medical condition of a subject is controlled.

In certain aspects, the invention relates to the herein provided method to predict the mortality risk and patient outcome of a subject, wherein the extracellular volume status of said subject is determined by the herein provided method. In certain aspects, the invention relates to a method used as a warning system for physician and clinicians to take appropriate therapy actions immediately, wherein said extracellular volume status of said subject is determined by the herein provided method.

In certain aspects, the invention relates to the herein provided method to predict organ failure, disregulated lymphatic flow activity, kidney dysfunction, decreased function or risk for cardiac dysfunction of a subject, wherein said extracellular volume status of said subject is determined by the herein provided method.

In certain aspects, the invention relates to the herein provided method for the use in treatment of subject suffering from a disorder or a medical condition that is selected from the group comprising water overload, edema, brain damage, post-aneurysm rupture, severe head injury, neurological impairment, severe multiple traumatic injuries, post-operative, cardiac risk, kidney injury, organ failure, disregulated lymphatic flow activity, kidney dysfunction, cardiac dysfunction, disease associated with disordered fluid balance. The terms “treatment”, “therapy” and the like are used herein to generally mean obtaining a desired pharmacological and/or physiological effect. The effect may be prophylactic in terms of completely or partially preventing a disease/medical condition/disorder or symptom thereof and/or may be therapeutic in terms of partially or completely curing a disease/medical condition/disorder and/or adverse effect attributed to the disease/medical condition/disorder. The term “treatment” as used herein covers any treatment of a disease/medical condition/disorder in a subject and includes: (a) preventing and/or ameliorating the disease/medical condition/disorder in a subject which may be predisposed to the disease/medical condition/disorder; (b) inhibiting the disease/medical condition/disorder, i.e. arresting its development; or (c) relieving the disease/medical condition/disorder, i.e. causing regression of the disease/medical condition/disorder. For example, the herein provided method can be used to control the therapy/treatment of a resuscitation patient. Thus, for example, the herein provided method can be employed to control the fluid management of a subject. The herein provided method can also be used to control the intravenous fluid administration in order to balance the fluid balance and/or salt balance in a subject to avoid a positive fluid balance, which is associated with an increased mortality rate (Acheampong et al., 2015). In certain aspects, the herein provided method can also be used to assess and control the fluid management of a subject to avoid a fluid shifting toward the interstitial space at a pathological amount and/or an overload in volume expansion. An overload in volume expansion that is considered as critical is, for example, more than 4 L within one day, within two days, within three days, within four days, within five days and or, preferably, within seven days 7.

In certain aspects of the invention, the herein provided method comprises:
(a1) comparing said level of proADM or a fragment thereof, preferably MR-proADM, to reference data corresponding to said level of proADM or said fragment thereof, preferably MR-proADM, of at least one reference subject; or
(a2) comparing said level of proADM or a fragment thereof, preferably MR-proADM, to data corresponding to said level of proADM or said fragment thereof, preferably MR-proADM, of the same subject obtained from prior analysis;
(b) identifying the fluid balance, salt balance and or globular volume status of said subject based on the comparison step (a), wherein the fluid balance, salt balance and/or the globular volume status of said subject is used to predict the mortality risk and patient outcome of a subject and/or is used for the assessment and control of the fluid management of the subject.

In the context of in vitro diagnosis, prognosis, risk assessment, risk stratification, therapy control and/or operative control of a disorder or medical condition in a subject and/or a patient, if the level of proADM or a fragment thereof, preferably MR-proADM is at least 0.5 nmol/L, for example, at least 0.5 nmol/L, at least 0.75 nmol/L, or at least 1 nmol/L and the subject has an increase in the fluid balance of at least 4.0 L (gain of 4 L of water per hospitalization), the subject has a positive fluid balance (increased fluid balance, i.e., a gain of the water content) that is considered as critical. In other words, in a critical subject, the positive fluid balance is at least 4 L, i.e., the gain of water is at least 4 L in a subject with a critical health status.

In preferred aspects, the method comprises determining in a sample the level of proADM or a fragment thereof, preferably MR-proADM, wherein the level of proADM or a fragment thereof, preferably MR-proADM of the subject is at least 0.5 nmol/L, for example, at least 0.5 nmol/L, at least 0.75 nmol/L, or at least 1 nmol/L, wherein said level of proADM or a fragment thereof, preferably MR-proADM indicates that said subject has a positive fluid balance, wherein said positive fluid balance is at least 4 L and wherein said positive fluid balance indicates that the subject has a critical health condition. In other words, said positive fluid balance indicates that said subject has a critical extracellular volume status.

In certain aspect, if the level of proADM or a fragment thereof, preferably MR-proADM is at least 0.5 nmol/L, for example at least 0.5 nmol/L, at least 0.75 nmol/L, or at least 1 nmol/L and the subject has an increase in the salt balance of at least 36 g (gain of 36 g of sodium or salt), the subject has a positive salt balance that is considered as critical. In most preferred aspects, the increase of sodium is at least 36.0 g and wherein said change indicates that the subject has a positive fluid balance and/or salt balance that is considered as critical. In other words, said positive salt balance and/or fluid balance indicates that said subject has an extracellular volume status that is considered as critical. In other words, in a critical subject, the positive salt balance (increased salt balance, i.e., a gain of the salt amount) is at least 36 g, i.e., the gain of salt is at least 36 g in a critical subject.

In other aspects, the method comprises determining in a sample the level of proADM or a fragment thereof, preferably MR-proADM, wherein the level of proADM or a fragment thereof, preferably MR-proADM of the subject is at least 1 nmol/L, wherein said level of proADM or a fragment thereof, preferably MR-proADM indicates that said subject has a positive salt balance, wherein said positive salt balance is at least 36 g and wherein said positive salt balance indicates that the subject has a critical health condition.

It is herein understood that further markers and/or parameters, i.e., in addition to proADM or a fragment thereof, preferably MR-proADM, improve the prediction of the fluid and/or salt balance. Therefore, in certain aspects, the method comprises determining the level of proADM or a fragment thereof, preferably MR-proADM in the sample, the body mass index of the subject, the weight of the subject, the age of the subject, the sex of the subject, the level of hemoglobin in the sample and the level of the total serum protein in the sample, wherein based on said markers and said parameters the fluid balance and/or the salt balance is determined, wherein a salt balance of at least 36 g and/or a fluid balance of 4 L indicate that the subject has a critical health condition. In preferred aspects, a salt balance of at least 36 g and a fluid balance of 4 L indicate that the subject has a critical health condition.

As used herein, a “critical state”, “critical health status”, “critical ill patient” or “critical subject” means that the subject or patient is in a life threatening situation as the extracellular volume status is considered as critical. As described above, subjects with a positive fluid balance and/or salt balance have an increased mortality rate. For example, a subject can be considered to have a critical health status, if it has an overload of fluid or salt, e.g., induced by excessive intravenous infusion. Therefore, a critical subject has a critical positive fluid balance (e.g. at least 4L), a critical positive salt balance (e.g., at least 36 g) and/or critical globular volume status (below 20 ml/kg). In certain aspects, a subject can be considered to have a critical health status, if it has a low globular volume status, e.g., lower than about 20 ml/kg or preferably lower than about 15 ml/kg. In certain aspects, a critical globular volume status is a globular volume below about 15 ml/kg.

The levels of the markers and/or parameters determined herein are a warning sign for the physician to take appropriate actions immediately. As used herein, a “critical extracellular volume status” refers to an increased or high extracellular volume. In preferred aspects of the invention, the increased extracellular volume is at least 3 L, preferably at least 4 L, wherein said increased extracellular volume identifies a subject having a critical health status. It is envisaged herein that the gain of fluid or the gain of salt, which increases the mortality rate of a subject, is also dependent on the subject characteristics, e.g., weight age or sex etc. For example, a positive fluid balance of 4 L determined in a heavy male subject has a different influence compared to a positive fluid balance in small kid. Therefore, it is envisaged herein that the gain of fluid and/or the gain of salt, i.e., 4 L or 36 g, respectively, that indicates a critical subject is dependent on the subject characteristics and can be higher or lower than 4 L or 36 g, respectively, dependent on the subject characteristic.

It is documented in the appended examples that there is a strong statistical relationship between the joint predictor, i.e., combining the fluid balance and salt balance, and the determination of critical ill patients. In certain aspects of the invention, the herein provided method determines critical ill patients, wherein the increase of water is at least 4 L and the increase of salt is at least 36 g Therefore, in preferred aspects, the method comprises determining in a sample the level of proADM or a fragment thereof, preferably MR-proADM, wherein the level of proADM or a fragment thereof, preferably MR-proADM of the subject is at least 1 nmol/L, wherein said level of proADM or a fragment thereof, preferably MR-proADM indicates that said subject has a positive salt balance and a positive fluid balance, wherein said positive salt balance is at least 36 g and said positive fluid balance is at least 4 L and wherein said positive salt balance and said positive fluid balance indicate that the subject has a critical extracellular volume status.

The appended examples demonstrate that there is a strong relationship between the sequential organ failure score (SOFA score) and the salt balance and/or fluid balance; see Examples 1 and 3. Therefore, in preferred aspects, the sequential organ failure assessment score (SOFA score) is determined based on the fluid balance and/or salt balance. In further preferred aspects, the sequential organ failure assessment score (SOFA score) is determined based on the fluid balance and/or salt balance, wherein the fluid balance and/or salt balance is determined based on the level of proADM or a fragment thereof, preferably MR-proADM. Thus, the method herein provided determines the level of proADM or a fragment thereof, preferably MR-proADM in the sample, wherein based on the level of proADM or a fragment thereof, preferably MR-proADM the SOFA score is determined.

In certain aspects, a SOFA score above 14 indicates a very severe health status indicating a critical health status of the subject. A SOFA score between 0 and 6 indicates a less severe health status and a SOFA score of 7 to 14 indicates a severe health status. In certain aspects, an increased level of proADM or a fragment thereof, preferably MR-proADM indicates the SOFA score of the subject, wherein the SOFA score above 14 indicates that the subject has a critical health status.

It is shown in the appended examples, that the inclusion of further parameters such as age, BMI and sex improve the predictive power to determine the SOFA score; see FIG. 5. Thus, in certain aspects, the herein provided method further comprises determining at least one parameter consisting of age, body mass index and sex.

It is envisaged herein that the sequential organ failure assessment score (SOFA score) is at least 15 and wherein said score indicates that the subject has a positive fluid balance and/or salt balance that is considered as critical.

As used herein, the “sequential organ failure assessment score” or “SOFA score” is one score used to track a patient's status during the stay in an intensive care unit (ICU). The SOFA score is a scoring system to determine the extent of a person's organ function or rate of failure. The score is based on six different scores, one each for the respiratory, cardiovascular, hepatic, coagulation, renal and neurological systems. Both the mean and highest SOFA scores being predictors of outcome. An increase in SOFA score during the first 24 to 48 hours in the ICU predicts a mortality rate of at least 50% up to 95%. Scores less than 9 give predictive mortality at 33% while above 14 can be close to or above 95%. The score tables below only describe points-giving conditions (Vincent JL et al. The SOFA (Sepsis-related Organ Failure Assessment) score to describe organ dysfunction/failure. Intensive Care Med. 1996; 22:707-710). In cases where the physiological parameters do not match any row, zero points are given. In cases where the physiological parameters match more than one row, the row with most points is picked. It assists doctors, nurses, and other members of the patient's health care team in estimating the risk of morbidity and mortality due to sepsis.

Tables 1 and 2: SOFA score table-scoring scheme

| Score SOFA | 0 | 1 | 2 | 3 | 4 |
|---|---|---|---|---|---|
| Respiratory | >400 | ≤400 | ≤300 | ≤200 with | ≤100 with |
| PaO2/FIO2 | | | | Artificial | Artificial |
| | | | | Ventilation. | Ventilation. |
| Coagulation | >150 | ≤150 | ≤100 | ≤50 | ≤20 |
| platelets | 10³/mm³ | 10³/mm³ | 10³/mm³ | 10³/mm³ | 10³/mm³ |
| Liver | <20 | 20-32 | 33-101 | 102-204 | >204 |
| Bilirubin | μmol/L | μmol/L | μmol/L | μmol/L | μmol/L |
| Cardiovascular | absence | MAP <70 | Dopa ≤5, or | Dopamin >5, Or | Dopamin >15, Or |
| Hypotension | | mmHg | Dobutrex | Epinephrin ≤0.1, Or | Epinephrin >0.1, Or |
| | | | | Norepinephrin ≤0.1 | Norepinephrin >0.1 |
| Central Nervous | 15 | 13-14 | 10-12 | 6-9 | <6 |
| System. GCS | | | | | |
| Kidney | <110 | 110-170 | 171-299 | 300-440 or <500 | >440 or <200 |
| creatinine or | μmol/L | | | ml/day | ml/day |
| diuresis | | | | | |
| Total = . . . | | | | | |
| Organ | 0 | 1 | 2 | 3 | 4 |
| Respiratory | 20% | 27% | 32% | 46% | 64% |
| Cardiovascular | 22% | 32% | 55% | 55% | 55% |
| Coagulation | 35% | 35% | 35% | 64% | 64% |
| Central nervous system | 26% | 35% | 46% | 56% | 70% |
| liver | 32% | 34% | 50% | 53% | 56% |
| kidney | 25% | 40% | 46% | 56% | 64% |

It is shown in the appended Examples 1 and 4 that the combination of the fluid balance and salt balance can predict efficiently (AUC>0.92) whether a subject will face a critical condition, e.g., has a positive fluid balance and/or a positive salt balance. In certain aspects of the invention, the herein provided method identifies a subject that has a critical health status based on the fluid balance and sodium balance of the subject.

The appended examples also demonstrate that the combination of the fluid balance and salt balance can predict efficiently the edema risk of a subject, i.e., the combined detection of fluid and salt balance can identify a critical ill patient with a risk for developing edema. In certain aspects of the invention, the herein provided method identifies a subject that has a critical edema risk based on the fluid balance and sodium balance. Thus, in certain aspects of the invention, the herein provided method is used to control the therapy of a subject that has a critical edema risk, wherein the edema risk is determined based on the fluid balance and sodium balance. In certain aspects of the invention, the herein provided method is used to control the therapy of a subject that has a critical edema risk, wherein the edema risk is identified based on the fluid balance and sodium balance, wherein the fluid balance and/or the salt balance is determined based on the level of proADM or a fragment thereof, preferably MR-proADM.

As mentioned herein in the context of proteins or peptides, the term “fragment” refers to smaller proteins or peptides derivable from larger proteins or peptides, which hence comprise a partial sequence of the larger protein or peptide. Said fragments are derivable from the larger proteins or peptides by deletion of one or more of amino acids from the larger protein or peptide.

As used herein, terms such as “marker”, “surrogate”, “prognostic marker”, “factor” or “biomarker” or “biological marker” are used interchangeably and relate to measurable and quantifiable biological markers (e.g., specific enzyme concentration or a fragment thereof, specific hormone concentration or a fragment thereof, or presence of biological substances or a fragment thereof) which serve as indices for health- and physiology-related assessments, such as a disease/disorder/clinical condition risk. Furthermore, a biomarker is defined as a characteristic that is objectively measured and evaluated as an indicator of normal biological processes, pathogenic processes, or pharmacologic responses to a therapeutic intervention. A biomarker may be measured on a biosample (as a blood, plasma, urine, or tissue test).

As used herein, a parameter is a characteristic, feature, or measurable factor that can help in defining a particular system. A parameter is an important element for health- and physiology-related assessments, such as a disease/disorder/clinical condition risk. Furthermore, a parameter is defined as a characteristic that is objectively measured and evaluated as an indicator of normal biological processes, pathogenic processes, or pharmacologic responses to a therapeutic intervention. An exemplary parameter can be selected from the group consisting of body mass index, weight, age, sex, IGS II, liquid intake, Acute Physiology and Chronic Health Evaluation II (APACHE II), World Federation of Neurosurgical Societies (WFNS) grading, Glasgow Coma

Scale (GCS) and sequential organ failure assessment score (SOFA score).

For the purposes of the present invention the “subject” (or “patient”) may be a vertebrate. In the context of the present invention, the term “subject” includes both humans and animals, particularly mammals, and other organisms. Thus, the herein provided methods are applicable to both human and animal subjects. Accordingly, said subject may be an animal such as a mouse, rat, hamster, rabbit, guinea pig, ferret, cat, dog, chicken, sheep, bovine species, horse, camel, or primate. Preferably, the subject is a mammal. Most preferably the subject is human. In the meaning of the invention, any sample collected from cells, tissues, organs, organisms or the like can be a sample of a patient to be diagnosed. As it is shown in the appended examples, the extracellular volume status of subjects suffering from various disorders or diseases can be predicted. Therefore, the method provided herein can be used on any subject that is a healthy subject or a subject that suffers from any disease or disorder. In preferred aspects, the subject suffers from a disease or disorder, wherein the disease or disorder is selected from the group consisting of edema, brain damage, post-aneurysm rupture, head injury, neurological impairment, multiple traumatic injuries, post-operative, organ failure, disregulated lymphatic flow activity, kidney dysfunction, cardiac dysfunction, disease associated with disordered fluid balance. In more preferred aspects of the invention, the subject suffers from a brain injury, an aneurysm, a head injury and/or multiple traumatic injuries and/or wherein said subject is post-operative. In most preferred aspects, the subject suffers from a severe brain trauma (SBT), an aneurysmal subarachnoid haemorrhage (SAH), severe trauma without head trauma (PT), post-surgical peritonitis with shock (P) and/or post digestive peritonitis surgery.

As used herein, the term “multiple traumatic injuries”, “multiple trauma”, “polytrauma” or “multitrauma” in the context of the invention encompasses a condition with two or more severe injuries in at least two areas of the body or a condition with a multiple injury, i.e. two or more severe injuries in one body area. Polytrauma may be accompanied with traumatic shock and/or hemorrhagic hypotensis and a serious endangering of one or more vital functions. At least one out of two or more injuries or the sum total of all injuries endangers the life of the injured subject with polytrauma. A trauma is an injury or damage to a biological organism caused by physical harm from an external source. Major trauma is an injury that can potentially lead to serious long-term outcomes like chronic pain.

As used herein brain injury is an injury of the brain, e.g., a traumatic brain injury. Brain injury occurs when an external force traumatically injures the brain. Head injury usually refers to brain injury, but is a broader category because it can involve damage to structures other than the brain, such as the scalp and skull. An aneurysm or aneurism is a localized, blood-filled balloon-like bulge in the wall of a blood vessel. Aneurysms can occur in any blood vessel, with examples including aneurysms of the circle of Willis in the brain, aortic aneurysms affecting the thoracic aorta, and abdominal aortic aneurysms. Aneurysms can also occur within the heart itself.

As used herein, a post-operative subject is subject that had a surgery. More preferably, the post-operative subject is a subject that had a major surgery. A major surgery can be any operation within or upon the contents of the abdominal, pelvic, cranial or thoracic cavities; or which, given the locality, condition of patient, level of difficulty or length of time to perform, constitutes a hazard to life or function of an organ or tissue. Major surgery usually requires general anesthesia, a period of hospitalization of varying length (often a week) and may be performed by a general -board-certified-surgeon in a secondary care hospital, or by a surgical subspecialist in a tertiary care hospital. More preferably, a post-operative subject is subject following a digestive surgery. More preferably, the post-operative subject is a subject that had major surgery and which suffers of a life threatening disease or disorder. This disease or disorder may be caused by the surgery itself. Most preferably, the post-operative subject is subject suffering from peritonitis with shock.

As used herein, a statistical relationship between the level of a marker(s), e.g., proADM or a fragment thereof, preferably MR-proADM, and/or parameter(s) with the extracellular volume status, e.g., the extracellular volume, blood volumes, or disorder(s)/disease(s)/clinical condition(s), of a subject was assessed employing statistical methods as shown in the herein appended examples. As demonstrated in the appended examples, random forest analysis (Breiman, 2001 and 2002; and Boulesteix et al. (2012); importance analysis; forward selection; linear regressions; leave-one-out; “R²” or “r²” (coefficient of determination); AUC (area under the curve); and survival analysis was employed. Any corresponding and suitable algorithm and software package available in the prior art can be used to calculate and analyze a statistical relationship between the parameters/values.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of and “consisting essentially of”.

Thus, the terms “comprising”/“including”/“having” mean that any further component (or likewise features, integers, steps and the like) can/may be present.

The term “consisting of” means that no further component (or likewise features, integers, steps and the like) is present.

The term “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

Thus, the term “consisting essentially of” means those specific further components (or likewise features, integers, steps and the like) can be present, namely those not materially affecting the essential characteristics of the composition, device or method. In other words, the term “consisting essentially of (which can be interchangeably used herein with the term “comprising substantially”), allows the presence of other components in the composition, device or method in addition to the mandatory components (or likewise features, integers, steps and the like), provided that the essential characteristics of the device or method are not materially affected by the presence of other components.

The term “method” refers to manners, means, techniques and procedures for accomplishing a given task including, but not limited to, those manners, means, techniques and procedures either known to, or readily developed from known manners, means, techniques and procedures by practitioners of the chemical, biological and biophysical arts.

The term “about” preferably refers to ±10% of the indicated numerical value, more preferably to ±5% of the indicated numerical value, and in particular to the exact numerical value indicated.

As used herein, the term “about” refers to ±10% of the indicated numerical value, and in particular to ±5% of the indicated numerical value. Whenever the term “about” is used, a specific reference to the exact numerical value indicated is also included. If the term “about” is used in connection with a parameter that is quantified in integers, such as the number of nucleotides in a given nucleic acid, the numbers corresponding to ±10% or ±5% of the indicated numerical value are to be rounded to the nearest integer. For example, the expression “about 25 amino acids” refers to the range of 23 to 28 amino acids, in particular the range of 24 to 26 amino acids, and preferably refers to the specific value of 25 amino acids.

Unless otherwise indicated, established methods of recombinant gene technology were used as described, for example, in Sambrook, Russell “Molecular Cloning, A Laboratory Manual”, Cold Spring Harbor Laboratory, N.Y. (2001)) which is incorporated herein by reference in its entirety.

The sensitivity and specificity of a diagnostic and/or prognostic test depends on more than just the analytical “quality” of the test, they also depend on the definition of what constitutes an abnormal result. In practice, Receiver Operating Characteristic curves (ROC curves), are typically calculated by plotting the value of a variable versus its relative frequency in “normal” (i.e. apparently healthy individuals not having a prenatal disorder or condition) and “disease” populations. For any particular marker (like MR-proADM), a distribution of marker levels for subjects with and without a disease/condition will likely overlap. Under such conditions, a test does not absolutely distinguish normal from disease with 100% accuracy, and the area of overlap might indicat where the test cannot distinguish normal from disease. A threshold is selected, below which the test is considered to be abnormal and above which the test is considered to be normal or below or above which the test indicates a specific condition. The area under the ROC curve is a measure of the probability that the perceived measurement will allow correct identification of a condition. ROC curves can be used even when test results do not necessarily give an accurate number. As long as one can rank results, one can create a ROC curve. For example, results of a test on “disease” samples might be ranked according to degree (e.g. 1=low, 2=normal, and 3=high). This ranking can be correlated to results in the “normal” population, and a ROC curve created. These methods are well known in the art; see, e.g., Hanley et al. 1982. Radiology 143: 29-36. Preferably, a threshold is selected to provide a ROC curve area of greater than about 0.5, more preferably greater than about 0.7, still more preferably greater than about 0.8, even more preferably greater than about 0.85, and most preferably greater than about 0.9. The term “about” in this context refers to +/−5% of a given measurement. The horizontal axis of the ROC curve represents (1-specificity), which increases with the rate of false positives. The vertical axis of the curve represents sensitivity, which increases with the rate of true positives. Thus, for a particular cut-off selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

In other embodiments, a positive likelihood ratio, negative likelihood ratio, odds ratio, or hazard ratio is used as a measure of a test's ability to predict risk or diagnose a disorder or condition (“diseased group”). In the case of a positive likelihood ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In the case of a negative likelihood ratio, a value of 1 indicates that a negative result is equally likely among subjects in both the “diseased” and “control” groups; a value greater than 1 indicates that a negative result is more likely in the test group; and a value less than 1 indicates that a negative result is more likely in the control group.

In the case of an odds ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group.

In the case of a hazard ratio, a value of 1 indicates that the relative risk of an endpoint (e.g., death) is equal in both the “diseased” and “control” groups; a value greater than 1 indicates that the risk is greater in the diseased group; and a value less than 1 indicates that the risk is greater in the control group.

The skilled artisan will understand that associating a diagnostic or prognostic indicator, with a diagnosis or with a prognostic risk of a future clinical outcome is a statistical analysis. For example, a marker level of lower than X may signal that a patient is more likely to suffer from an adverse outcome than patients with a level more than or equal to X, as determined by a level of statistical significance. Additionally, a change in marker concentration from baseline levels may be reflective of patient prognosis, and the degree of change in marker level may be related to the severity of adverse events. Statistical significance is often determined by comparing two or more populations, and determining a confidence interval and/or a p value; see, e.g., Dowdy and Wearden, Statistics for Research, John Wiley & Sons, New York, 1983. Preferred confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001.

The present invention is further described by reference to the following non-limiting figures and examples.

### DESCRIPTION OF FIGURES

FIG. 1. Distribution of body water, wherein the body water can be divided in the extracellular volume (part in ellipse) and the intracellular volume (corresponding to about 57%). The extracellular volume by itself can be further divided in blood volume, i.e., the globular volume (corresponding to about 6%) and the plasma (corresponding to about 6%), and the interstitial volume (corresponding to about 27%).

FIG. 2. Deming regression of MR-proADM (logarithmic scale) and fluid balance (delta H2O) (A) and salt balance (delta Na) (B).

FIG. 3. Box-and-Whisker blot of MR-proADM concentration in nmol/L of intensive care unit patients suffering aneurysmal subarachnoid haemorrhage (SAH), severe trauma without head trauma (PT), severe brain trauma (SBT) or post-surgical peritonitis with shock patients (P) on day 2 (A), day 5 (B) and day 7 (C). Mean concentration of MR-proADM in nmol/L is shown for day 2, day 5 and day 7 (D).

FIG. 4. ROC plot for MR-proADM for the prediction of the fluid balance (delta H2O) (A) and the salt balance (delta Na) (B) in intensive care unit patients suffering aneurysmal subarachnoid haemorrhage, severe trauma without head trauma, severe brain trauma or post-surgical peritonitis with shock

FIG. 5. Predicted SOFA (leave-one-out). Patients are sorted by increasing SOFA. The solid black line gives the true SOFA values. Patient id on x-axis (patient begin sorted by increasing SOFA value), predicted SOFA on y-axis. The solid black line gives the (increasing) true SOFA value for all patients. The blue circles give the predicted SOFA.

FIG. 6. Predicted delta.H2O, delta.Na and P-critical for 201 patients. The 126 “regular” patients (patients without edema) are represented by empty circles, and the 75 “critical” patients (patient with edema) are represented by full circles.

FIG. 7. Random forest variable importance for globular volume reference model.

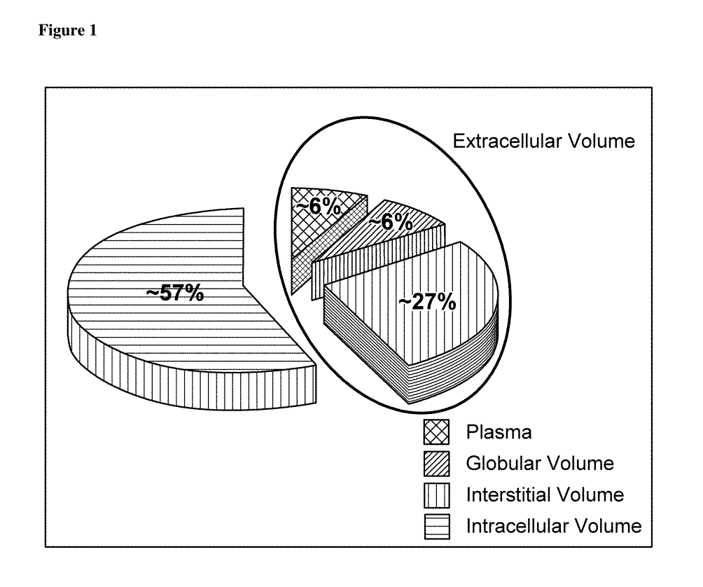

```
Sequences
SEQ ID NO: 1: amino acid sequence of pre-pro-ADM:
    1 MKLVSVALMY LGSLAFLGAD TARLDVASEF RKKWNKWALS RGKRELRMSS
  51 SYPTGLADVK AGPAQTLIRP QDMKGASRSP EDSSPDAARI RVKRYRQSMN
101 NFQGLRSFGC RFGTCTVQKL AHQIYQFTDK DKDNVAPRSK ISPQGYGRRR
151 RRSLPEAGPG RTLVSSKPQA HGAPAPPSGS APHFL
SEQ ID NO: 2: amino acid sequence of MR-pro-ADM
(AS 45-92 of pre-pro-ADM):
ELRMSSSYPT GLADVKAGPA QTLIRPQDMK GASRSPEDSS PDAARIRV
```

The Examples have been performed by detecting MR-proADM. However, as outlined herein above, the invention can also be performed by detecting proADM or another peptide fragment thereof.

The following non-binding Examples illustrate the invention.

### EXAMPLES 1

### Positive Fluid Balance, Blood Volumes and MR pro-ADM in Critically Ill Patients

Methods

Patients and Procedures

This prospective 7-day observational study was conducted from March 2012 to September 2014 in a 30-bed Department of Anaesthesiology and Intensive Care at Bicetre University Hospital in France. The Institutional Review Board of Bicetre hospital approved the study on December 2011 and all patients or their relatives signed an inform consent. Four types of patients were studied: patients with severe brain trauma (SBT), aneurysmal subarachnoid haemorrhage (SAH), severe trauma without head trauma (PT) and post-surgical peritonitis with shock (P). Patients were included if they needed to be mechanically ventilated in D2 (D2) permanently. SBT was defined as a brain trauma with a Glasgow Coma scores of less than 9 (GCS<9). SAH were included when the score in WFNS scale was 4 or 5 (Brisman et al., 2006)]. PT were included when ISS score was 25 or more. P were included after abdominal surgery with signs of shock with hemodynamic complications (hypotension, low cardiac output) or lactate >4 mmol/L and the prescription of catecholamine at admission in intensive care.

The exclusion criteria were age <18 years, pregnancy and chronic cardiac insufficiency (NYHA II or IV).

General and demographic data were collected: age, sex, weight and height for BMI, IGS II, ISS, admission date, Glasgow outcome score (GOS) before leaving the intensive care. Sequential Organ Failure Assessment (SOFA) scores were measured at the arrival (D0), D2, D5 and D7 (Vincent et al., 1996).

Every day, the mean arterial pressure (MAP) and the dose of norepinephrine, if used, were noted for each patient. Biological parameters were measured: haemoglobin concentration [Hb] and plasmatic proteins, plasmatic and urinary concentrations of Na⁺, K⁺, Cl⁻ urea, creatinine and osmolarity. Biological urinary results obtained in the morning from the total 24 hour diuresis were used to calculate the sum of the urinary loss of the previous day of Na+, K+, urea and clearance of creatinine. The weight and temperature were measured at day 2 (D2), day 5 (D5) and day 7 (D7).

A combination of echography signs, signs of fluid responsiveness in ventilated patients and repeated measurements of cardiac filling guided daily fluid administration (Feissel et al., 2004; Feissel et al., 2001; Monnet et al., 2013; and Gore et al., 2005). Moreover, the amount of intravenous fluid given was also determined by a number of variables including heart rate, arterial pressure, blood lactate level and cardiac output.

Evaluation of Extracellular Volume

Every day, the salt and fluid balance was calculated to estimate the change in extracellular space. Every morning, a complete input-output assessment of the previous day was done for salt and water. The exact salt and water contributions were noted. All losses were measured: diuresis, ileostomy and ventricular drainage if required. The loss of sodium (Na³⁰ ) was measured from liquids and deducted from the salt contribution. The difference of input water (enteral nutrition and sum of the day of crystalloids or colloids infusion) and loss of water was also calculated. Insensitive losses were estimated as a function of the body temperature. The gain or loss of Na⁺(ΔNa⁺) and water (ΔH₂O) were calculated each day and summed to the result of the day before as cumulative fluid balance. The clearance of creatinine was also calculated each day. All calculations for each patient were made by a doctor and verified by a second doctor (BV, PEL and HF).

Blood Volume Measurements

The total blood volume with red blood cells marked with chrome 51 (Cr₅₁) was measured at D2 and D7. For practical reasons, D2 is not always exactly the second day after patient's admission but it is some time from day 1 to day 3. Day 7 is from day 6 to day 10.

In the laboratory, 10 mL of patient's own blood was radio-labeled with chrome 51 (Cr₅₁) and radioactivity marked red blood cells were carefully selected by careful removing of all plasmatic radioactivity. Then, a known quantity of radioactive red blood cells was re-injected in the total blood circulation and two samples in arterial line at 10 and 30 minutes were performed. The measure of the radioactivity of the two samples allowed to deduce the total blood volume (TBV) in mL or mL/Kg if using the patient's weight (Gore et al., 2005). Then, haematocrit number and the measured total blood volume defined the red blood cells volume (RBCV), (mL or mL/Kg) and plasmatic volume (PV), in mL or mL/Kg. The normal values (±20%) are 72±14 mL/Kg for TBV, 32±6 mL/Kg for RBCV and 40±8 mL/Kg for PV (Gore et al., 2005). At D7, the plasmatic volume (PVI₁₂₅) was directly measured with a known small amount of the radio-labelled albumin with iodine 125 (I₁₂₅) injected to the patient, and samples were collected at 10, 30 minutes and 2 hours (Fairbanks et al., 1996). The normal value of PV measured with I₁₂₅ is 45±10 mL/Kg (Gore et al., 2005). Usually, the plasmatic volume obtained with I₁₂₅-albumin is slightly larger than the plasmatic volume obtained from measurements with Cr51-red blood cells because of a greater volume of the distribution of albumin than that of the red blood cells (Gore et al., 2005).

When blood volume measurements were performed at D2 and D7, trans-thoracic echographic measurements were used to evaluate stressed volume with two indicators: variation of inferior vena cava (AIVC) and E/E′ (Vincent et al., 1996 and Feissel et al., 2004).

Biomarkers Analysis

For each patient, plasmatic biomarkers were studied at D2, D5 and D7. D2 and D7 were the days of blood volume measurements. D5 samples were always done exactly 2 days before D7. The Pro-adrenomedulin (MR pro-ADM), Pro-ANP, renin, angiotensin II, aldosterone, cortisol, adrenalin and epinephrine, CT-pro-arginine vasopressin (copeptin) and pro-endothelin, were measured as biomarkers, which potentially interfere with extracellular or plasmatic volumes and decrease (MR pro-ADM and Pro-ANP) or increase arterial pressure. The Erythropoietin (EPO) was measured for its capacity to change with RBCV. The standards and techniques employed are presented in Table 3.

TABLE 3
Methods, units and inferior and superior normal
values for all plasmatic biomarkers studied.

| | | | Sensi- | Lower | Higher |
|---|---|---|---|---|---|
| Parameters | Methods | Units | bility | level | level |
| Pro-ADM | Kryptor+ | nmol/l | 0.25 | | 0.39 |
| Pro-ANP | Kryptor+ | pmol/l | 4.5 | | 85.2 |
| Renin | immuno- | pg/ml | 1 | 3 | 16 |
| | luminescence | | | | |
| Angiotensin | Chroma- | pmol/l | 2 | 19 | 38 |
| II | tography + | | | | |
| | radio- | | | | |
| | immunology | | | | |
| Aldosteron | radio- | pg/ml | 10 | 42 | 201 |
| | immunology | | | | |
| Cortisol | immuno- | ng/dl | 1 | 9 | 22 |
| | luminescence | | | | |
| Epinephrin | HPLC | pg/ml | 20 | | 80 |
| Norepineprin | HPLC | pg/ml | 20 | | 450 |
| Copeptin | Kryptor+ | pmol/l | 0.5 | 1.1 | 16.4 |
| Pro- | Kryptor+ | pmol/l | 1 | | 44.3 ± 10.6 |
| endothelin | | | | | |
| EPO | ELISA | mUI/ml | 1.2 | 6.4 | 63.8 |

Statistical Analysis

Power analysis: The number of patients required was calculated using published range of values of Brain Natriuretic Peptide (BNP) and erythropoietin (EPO) concentration in similar patients (Dorhout Mees et al., 2011). An 80% power, with a 50% expected difference between groups were considered.

The normality of data distributions was checked using quantile-quantile (qq) plots and the Shapiro test. When data were log-normally distributed, statistical comparisons were performed on transformed data. The data is reported as mean ±standard deviation (SD) or median (25^{th} to 75^{th} percentiles) or count and frequency (percent).

Correlations were studied using linear or Spearman regression depending on the normality of data. In addition, since most data was measured with error, a Deming regression with equal variances was used to calculate the slope of the regression curve. Intra- and inter-group comparisons used ANOVA (factorial of for repeated measures) or non-parametric (Kruskal-Wallis or Friedman) tests followed by Tukey or Mann-Whitney/Wilcoxon tests, the latter corrected for multiple comparisons by the Bonferroni correction.

The receiver operating curves (ROC) were constructed to calculate the performance of the biomarkers for predicting the fluid and sodium balance. The best sensibility/specificity cut off was calculated using a non-weighted Youden index.

Finally, a concordance analysis using a four-quadrant plot with and without a “gray-zone” exclusion of 15% was used to compare the variations of markers and the fluid and sodium balance (Perrino et al., 1998).

Analysis was performed using R (The R Foundation for Statistical Computing, Vienna University of Technology, Vienna Austria at http://www.r-project.org/accessed 20 Jun. 2015). The statistical significance was set at P <0.05.

An independent link was found between the fluid balance and blood volumes, and the biomarkers. A mathematical tool of prediction was built using biomarkers and other significant parameters in order to determine clinically exploitable predictors of fluid state in critically ill patients using a selection of “reasonable” covariates. SOFA score, severity score for patient's condition, which is a multilevel ordered variable, are considered as an additional response variable.

For fluid balance (ΔNa⁺ and ΔH2O), a joint predictor was built to identify critically ill patients who have both ΔNa⁺>36 g and ΔH2O >4 L. For that propose, we first considered jointly the leave-one-out residuals for (ΔNa⁺ and ΔH2O) the distribution of which is found to be Gaussian (centred) with a covariance matrix. It is therefore possible to obtain for each couple of predicted values of (ΔNa⁺ and ΔH2O) a critical probability (Pcritical), easily computed using the mvtnorm package (Genz et al., 2009; and Genz et al., 2014).

Results

During the first 7 days after admission in ICU, 67 patients distributed between SBT (n=21), HSA (n=20), PT (n=20) and P (n=6) were studied. General demographic data and the number of patients studied in each clinical situation are shown in Table 4. SOFA scores at D0, D2, D5 and D7 are presented in Table 5

TABLE 4
General and demographic data. Data are presented
as mean ± standard deviation (M ± SD)
except for Glasgow Outcome scale (GOS) presented as median

| | Total | SBT | SAH | PT | P |
|---|---|---|---|---|---|
| | (n = 67) | (n = 21) | (n = 20) | (n = 20) | (n = 6) |
| Age (Years) | 46 ± 19 | 38 ± 16 | 53 ± 14 | 39 ± 18 | 69 ± 16 |
| Weight (kg) | 75 ± 18 | 73 ± 14 | 53 ± 14 | 84 ± 22 | 71 ± 9 |
| Height (cm) | 172 ± 10 | 178 ± 9 | 169 ± 9 | 173 ± 11 | 161 ± 8 |
| IGS II | 43 ± 13 | 49 ± 9 | 42 ± 12 | 37 ± 11 | 54 ± 10 |
| Sexe W/M | 24/43 | 18/3 | 11/9 | 6/14 | 4/2 |
| Length of | 27 ± 22 | 34 ± 3 | 27 ± 16 | 22 ± 13 | 21 ± 6 |
| stay (day) | | | | | |
| GOS | 4 (2) | 4 (1) | 4 (1) | 5 (1) | NP |

TABLE 5
SOFA score at D0, D2, D5 and D7 for each clinical group.

| | SBT | | SAH | | PT | | | |
|---|---|---|---|---|---|---|---|---|
| | (n = 21) | | (n = 20) | | (n = 20) | | P (n = 6) | |
| | MED | ET | MED | ET | MED | ET | MED | ET |
| SOFA D0 | 12 | 2 | 8 | 2 | 12 | 3 | 15 | 1 |
| SOFA D2 | 8 | 3 | 7 | 3 | 10 | 3 | 14 | 2 |
| SOFA D5 | 6 | 4 | 8 | 3 | 6 | 4 | 13 | 5 |
| SOFA D7 | 5 | 4 | 6 | 2 | 4 | 4 | 9 | 3 |

SBT: severe brain trauma,
SAH: sub arachnoid haemorrhage,
PT: polytrauma,
P: peritonitis

All patients were studied. The gains in extracellular volume are reported as changes in salt (ΔNa+) and changes in water (ΔH2O) at D2 and D7 for each clinical situation. Nearly all patients showed a positive fluid balance, i.e., an increase in extracellular volume at D2 (64/67 for ΔNa⁺ or 63/67 for ΔH₂O) and the majority at D7 (42/67 for ΔNa⁺ or 41/67 for ΔH₂O). A positive balance of hydro-containing soda at D2 and D7 is observed in all pathologies with a higher increase in salt for PT and P than for SHT and SAH. For example at D2, PT and P show a cumulative increase in salt of 70±32 g and 77±28 g as STB whereas SAH show 43±24 g and 28±24 g. ΔNa⁺ is related to ΔH₂O showing that retained water is closely bound to retained salt (r²=0.67; p<0.0001). As a known indicator of extracellular space, plasmatic concentration of proteins and variation in weight are related to ΔNa⁺ and ΔH₂0 but those relations are weak (r²=0.44 for plasmatic proteins and ΔNa⁺, r²=0.35 for plasmatic proteins and ΔH₂O, and r²=0.33 for weight and ΔH₂O). As plasmatic proteins, [Hb] have a weak relationship with ΔNa⁺ (r²=0.15) and ΔH₂0 (r²=0.24).

The measured volumes with Cr₅₁ (total blood volume (TBV), red blood cells volume (RBCV) and plasmatic volume (PV)) were studied in 62 patients at D2 and 63 patients at D7. In most patients a decrease in TBV was observed. Only 21 patients at D2 and 25 at D7 are in the normal 20% range. Hypovolemia, with TBV under 20% of normal values, existed for 46 patients (74%) at D2 and 42 (66%) at D7. In all patients, low RBCV were found except for 1 patient at D2 and 2 patients at D7. These patients with normal RBCV were all transfused. Even in non-haemorrhagic conditions (SBT or SAH), the decrease of RBCV could be significant with a lack of 50% or less of normal RBCV in 25 patients (40%) at D2 and 21 (33%) at D7. There is no statistical relationship between TBV or RBCV and fluid balance, i.e., ΔNa or ΔH2O.

The distribution of PV is in normal range. There is no relationship between PV and the fluid balance, i.e., ΔNa or ΔH2O, and we also found no relationship between changes in PV and changes in Δ H2O between D2 or D7. The change in PV is not related to changes in the plasmatic concentration of proteins. The Haemoglobin concentration is weakly related to RBCV (r²=0.33) and not related to VP (r²=0.026).

At D7, the VPs of 58 patients were measured by I125 (PVI₁₂₅). There is a statistical significant relationship between both PV (PV Cr51 and PVI₁₂₅). The Deming regression found a slope at 0.852 (CI 95% 0.610-1.08) and an intercept at 780 mL (CI 95% 103-1485 mL) (interval confidence is designated herein CI) in favor of PVI₁₂₅ with an r²=0.752.

No relationship between the blood volumes and the variations of the inferior vena cava were found (AIVC) or E/E′.

Biomarkers

The detailed kinetics of all biomarkers are given in Appendix I. Most of them are increased in D2 and, then, decreased statistically at D5 and D7 (Copeptin, Angio II, Renin); some decreased statistically only at D7 (MR pro-ADM, EPO). Other biomarkers are unchanged during the three considered days (Cortisol, Aldosterone, Pro-ANP, Pro-endothelin). The plasmatic norepinephrine is not pertinent to the study because of the bias due to the external infusion used as a treatment in patients.

Among all biomarkers tested, we found only two biomarkers, renin and MR pro-ADM, to have a statistical relationship with ΔNa⁺ and/or ΔH₂O (Table 6). The relationship between MR pro-ADM and ΔNa⁺ and/or ΔH₂O appears to be very strong and completely independent of the type of clinical situation.

TABLE 6
Probability of relation between plasmatic concentrations
of biomarkers and measure of fluid balance (p),
as paid by modelling for mixed purposes.

| Biomarkers | ΔH2O | ΔNa⁺ |
|---|---|---|
| Renin | 0.048 | 0.0001 |
| MR-proADM | <0.0001 | <0.0001 |

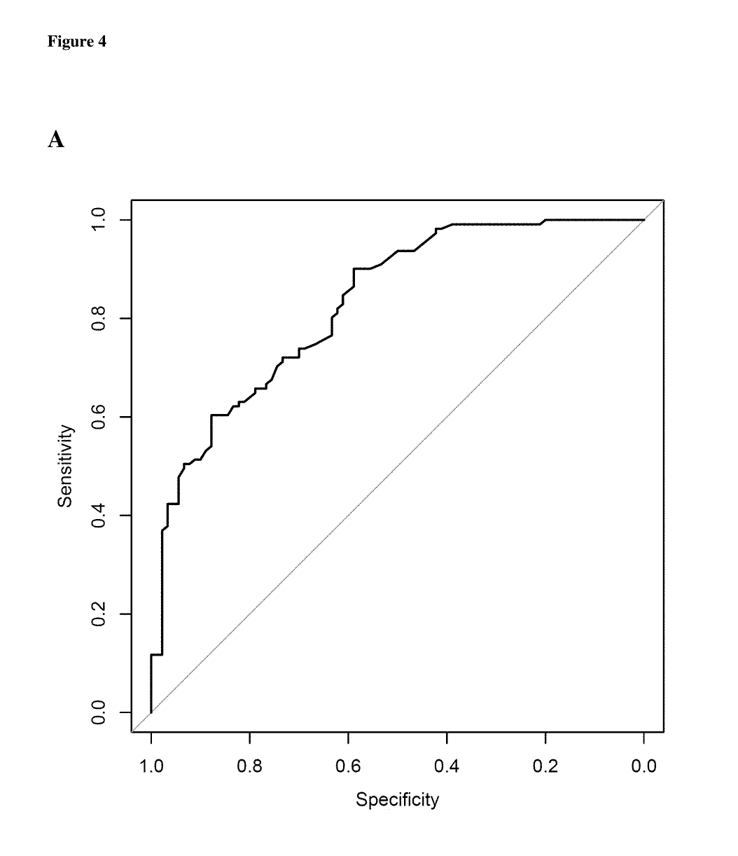

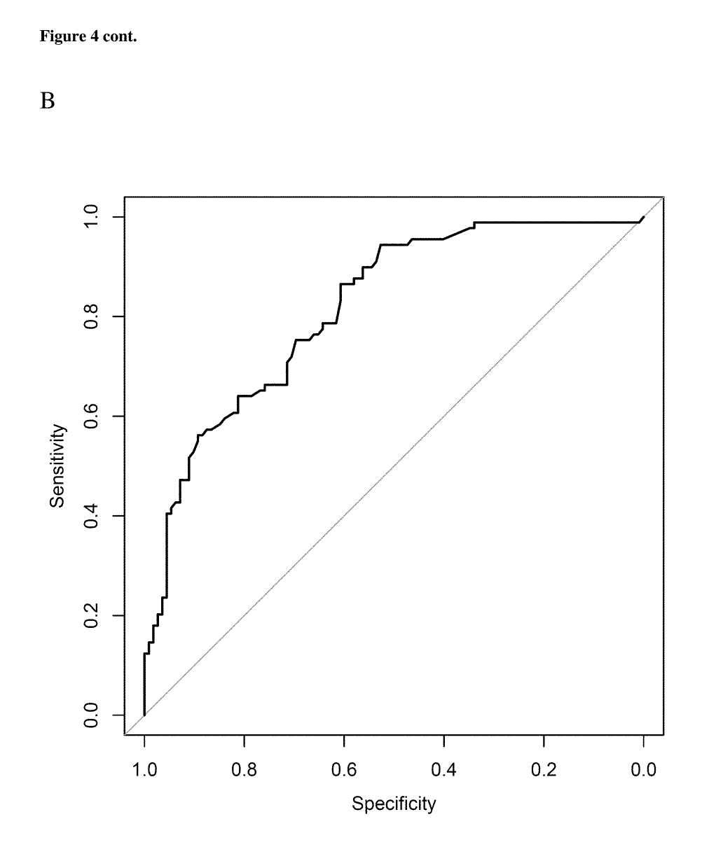

Then, 4 L for ΔH2O and/or 36 g for ΔNa⁺ (4 liters at 9%o give 36 g of salt) was considered as thresholds for positive fluid balance (Bjerregaard et al., 2005). An ROC curve allowed us to find the best threshold for MR-proADM suggesting the best compromise sensitivity/specificity with a not-balanced index of Youden (FIG. 4).

Because an acceptable probability was found that MR-proADM can predict salt balance and fluid balance (ΔNa⁺ and ΔH2O), a predictive score for Δ Na⁺ and/or ΔH₂O was built with MR-proADM and other simple covariates. For ΔNa⁺ and/or ΔH₂O, the best model for prediction needs, from the stronger parameter to the weakest, MR-proADM, BMI at D0, Weight at D0, age, sex, [Hb], IGS and Fluid intake at D0. Removing the MR-proADM of the model reduces the power of prediction. The two models can independently explain roughly 70% of the variance and have a good discriminative power with an AUC of 88% for ΔNa⁺ and 92% for ΔH₂O. Moreover, the absence of IGS and fluid intake at D0 have very limited consequences in this two models with only a loss of 2-3% of r² and no effect on the AUC.

If a joint predictor to determine critically ill patients who have both Δ Na⁺>36 g and ΔH₂O >4 L was built as described previously in the statistic paragraph, the performance was significantly improved. The discriminative power of this joint predictor is high with AUC=0.9987 (95% CI [0.9964-1]). For example, with a threshold of 0.4 on P-critical, a sensitivity of 0.988 and a specificity of 0.949 were obtained. Moreover, this fluid balance predictor is significantly bound to patients' SOFA scores.

Further, biomarkers were tested and blood volumes were measured. Interestingly, among all biomarkers, as for fluid balance, the same two biomarkers, renin and MR pro-ADM, have a statistical relationship with blood volumes. However, if the blood volumes were analyzed again by regression for mixed purposes on the logarithm of the biomarker values, predictive characters of the markers were weak with a low AUC of the ROC curve (for renin: AUC TBV=0.5798 and AUC PV=0.6159 and for MR pro-ADM: AUC TBV=0.5967 and AUC PV=0.6277). Moreover, and in spite of a high probability (p=0.0002), it was not possible to build an ROC curve and measure AUC for RBCV because the values were too uncharacteristic and low. In those situations, no thresholds and no predictive models were tested.

Discussion

This study has shown that a simple biomarker, MR-proADM, known as an indicator of endothelium permeability (Christ-Crain et al. 2005 and Koyama et al., 2013), is a good surrogate for the increase in salt and water in extracellular volume during the first week after admission of critically ill patients. In addition, no relationship was found between the increase in the salt balance or fluid balance and direct blood volumes measurements at D2 and D7.

It is now clear that excessive salt or fluid balance may be considered as a risk factor of morbidity and mortality in critically ill patients (Boyd et al., 2011; Kelm et al., 2015; Acheampong et al., 2015; and Malarian et al., 2014). This situation is frequent because the goal to have efficiency for cardiac output suggests performing fluid expansion to reach efficacy (Cecconi et al., 2011 and Vincent et al., 2011). Frequently in high inflammatory conditions in the first days after aggression (in sepsis, trauma or cerebral aneurysm), many patients are at high risk of accumulating salt with water in extracellular volume because excessive capillary permeability, increase in fluids, decrease in diuresis and difficulties to maintain salt in plasmatic volume. Patients respond differently to the fluid resuscitation. Accordingly, a clear and rapid surrogate marker is required to better stratify patients and to identify those with the positive salt balance in interstitium in order to personalize treatments: choosing more liquids to perfuse or adjustment with catecholamine or diuretics. This marker should also be independent of the kind of admission justification in intensive care.

If MR-proADM seems to be an especially good indicator of salt and fluid balance, it cannot be only an indicator of capillary permeability. The Na⁺ equilibrium is not only controlled by the kidney (Titze et al., 2014). The interstitial clearance of Na⁺ appears to be mediated by immune cells specially macrophages controlling Na⁺ expurgation via the interstitial lymph capillary system (Titze et al., 2014). In inflammation states, the loss of homeostatic immune cell control by macrophages involving failure in their capacity to clear Na⁺, may clarify the Na⁺ accumulation in interstitium (Jantsch et al., 2014). Interestingly, adrenomedullin peptide is known to play a role in lymph channels organization especially in organogenesis (Kahn et al., 2008).

The total amount of excess in interstitium is not only due to inflammation and decrease in Na⁺ clearance. It can be worsened by physiological causes such as elevated hydrostatic pressure provoked by high mean arterial pressure (MAP) or a high infusion rate (Bark et al., 2013) or an elevated blood stretch forced by an excessive cardiac output. A high MR-proADM (more than 1 nmol/1), indicates a soda overload, and a gain of for example more than 36 g of Na⁺ and 4 L of water, could be a warning sign for the physician to take appropriate actions immediately. In many intensive care units, nurses systematically measure fluid balance by daily weight or daily calculation of input and output of liquids. These methods are not precise. For example, a weak relationship was found between weight and fluid balance in our observation study (r²=0.33). Usual markers of extracellular volume, as plasmatic proteins or [Hb] have a weak relationship with salt and fluid balances (r²=0.35, and r²=0.24). In this study, the measures of salt and fluid balances needed many biological samples and was always done and controlled by two doctors. In every day practice, nurses cannot devote the time necessary to collect the required information. The MR-proADM seems to offer a more exact measure of salt balance and extracellular condition and can also be used as an emergency surrogate following the emergency of the situation. In some acute situations, in the first days after shock, timing can be crucial. In real life, the presence of overload is discovered too late often after organ damage (acute lung injury, abdominal compartment syndrome, renal insufficiency). The relationship between SOFA score and the salt and fluid balance prediction model reinforced the notion that MR-proADM can be an interesting bedside tool.

No markers were found to estimate the total blood volume (TBV), plasmatic volume (PV) or red blood cells volume (RBCV). Renin and MR-proADM were found significant but not enough to build a valuable model of prediction. Even, ΔNa⁺ or ΔH₂O are not predictors for blood volumes.

Another interesting result of this study is the demonstration that there is no relationship between PV and the fluid balance. This was particularly unexpected because the volume expansion is the main reason for fluids perfusion. Nevertheless, PV is the only volume where most of the patients are in normal ranges at D2 and D7. The absence of correlation between fluid balance and PV reinforced the hypothesis of Na and water trapped in interstitial volume. In daily practice, it will be useful to have a better control over this plasmatic volume but we detected no links with biomarkers nor proteins or [Hb] nor with signs related to stressed volume measured with echography. Other studies with a combination of biomarkers or others elements must be conducted to study this problem.

The correlation found between the two modes of measurement of PV (Cr₅₁ and I₁₂₅) at D7 reinforced the result identified herein. Albumin can have a larger distribution volume than red blood cell especially if capillary permeability is increased in pathological situations. At D7 the difference is rather strong (780 mL) suggesting that capillary permeability is not totally repaired. It may be interesting to discuss this comparison also at D2. Unfortunately, a measurement using I₁₂₅ at D2 could not be performed because of interactions with some methods for measuring biomarkers (Table 1).

Low RBCV, anaemia, is rather common and explains low results for TBV. The RBCV is difficult to assess and requires the CR₅₁ process and a radioactivity unit management. Its assessment is only done in rare cases such as the Waquez disease. In daily practice the [Hb] threshold test (<7-8 g/dl) is used to determine the need for blood transfusion. As [Hb] is the ratio between PV and RBCV, [Hb] is a poor surrogate for RBCV (Takanishi et al., 2008). Patients may have [Hb] at 11 g/dL and only 50% of normal RBCV. The lack of 50% of normal red blood cells may have an important impact in patient health. Some studies have suggested that the total amount of RBC can be a prognostic marker of on cognitive recuperation (Naidech et al., 2007). Nevertheless, RBC transfusion is not only the replacement of missing cells. In the present study, EPO is not dependant on the level of RBCV suggesting that EPO is not a good surrogate for RBCV and that EPO stimulation is not only explained by RBC quantity but also by RBC quality.

Clinical situations described in the study reflect the daily practice of our service. Severe trauma, brain injury and neurologic situations are frequent, septic shocks are less present. Nevertheless, the group as a covariate does not intervene in the prediction for fluid balance or blood volume suggesting that volume disorders are rather independent of the pathology. Other studies will be necessary to confirm the present results.

The overload following a shock is an important and under-appreciated factor of survival. Positive salt balance is actually documented as an important prognosis marker. Blood volumes are not automatically bound to volume expansion. MR-proADM is an interesting surrogate to evaluate salt and fluid balance in the first week after an acute inflammatory situation in critically ill patients A brake in the frequent excessive volume expansion is suggested.

### EXAMPLE 2

### Prediction of the Fluid Balance and/or Salt Balance by MR-proADM

1. Introduction

The objectives of this study are to answer the following questions:
Is it possible to predict the variations of Na and H₂O using biomarkers and/or other covariates?
Is it possible to predict volume responses using biomarkers and/or other covariates?

For that purpose, the predictive performance of the supplied biomarkers and covariates using datamining techniques and model selection were assessed.

2 Material and Methods

Please note that the following Material and Methods section describes the material and methods used in Examples 2 to 4 which serve only as exemplary embodiments.

2 Material and Methods

2.1 Data

Here follows a description of the variables and datasets used in this report. A 3.5% rate of missing data (54% for max.lactate) was observed, such a value is low. As a consequence, missing data was not really a major issue in this study. For this reason, we decided to impute all missing vales using the corresponding column mean.

Response Variables

A total of 2 response variables of interest were considered:
delta.H2O (D2, D5, D7): variation of water (H2O), expressed in liters (L). A variation ≥4.0 L is considered critical.
delta.Na (D2, D5, D7): variation of Sodium (Na), expressed in grams (g). A variation ≥36.0 g is considered critical.
VT (D2, D7): total volume, expressed in mL/kg. A total volume ≤60 mL/kg is considered critical (the classical threshold of 72 has been lowered to increase the number of controls).
VP (D2, D7): plasmatic volume, expressed in mL/kg. A plasmatic volume of ≤40 mL/kg is considered critical.
VG (D2, D7): globular volume, expressed in mL/kg. A globular volume of ≤15 mL/kg is considered critical (the classical threshold of 32 has been lowered to increase the number of controls).
SOFA (D2, D5, D7): Sequential Organ Failure Assessment score. A multilevel ordered response: the score grows with the severity of the patient's condition. Note that the 4 SOFA scores above 15 have been set to 15 in order to avoid SOFA scores with only one observation.

NB: for D7, the second volume measurements using iode instead of CR51 has been discarded.

Covariates
patient covariates (8): age, sex, weight.D0, bmi.D0 (body mass index), IGS.II (IGS II socre), GOS (Glasgow Outcome Scale), Fluid.D0 (liquid intake at D0), Na.D0 (sodium intake at D0)
daily covariates (5): max.temp (maximal temperature), max.lactate (lactate), min PAM (minimal of the mid arterial pressure), FC (heart rate), max.cathe (catecholamine)
biomarker covariates (11): Hb, Prot.D0 (total serum protein at day 0), Prot (total serum protein), Angio (angiotensin II), Renin, Aldo (aldosterone), Pro.ANP, Adre (adrenalin), Pro.Endo (pro-endothelin-1), CT.proAVP, MR.proADM, Cortisol, Nor (noradreanline), EPO (all in log scale, log 1p transform).

NB: bmi has been computed from height and weight using the formula: bmi.J0=weight.J0/(height.J0/100)2. Covariate height.D0 has been discarded to avoid redundancy with weight.D0 and bmi.D0.

2.2 Statistical Methods

All statistical computations have been done using the R software (Rmanual) version 3.0.2.

Random Forests

Random forests (Breiman, 2001; Breiman, 2002) were used to predict the response variables using covariates. The approach consists in building repetitively decision trees from bootstrapped data. A total of 50,000 trees are built for each run (500 for the leave-one-out procedure). This is a powerful datamining approach which is known to be able to capture even non linear effects. A good introduction to random forest in the biomedical context, see Boulesteix, 2012.

Importance

In order to rank covariates by decreasing importance, a sensitivity analysis was performed. For each covariates, random forest prediction was performed with or without the studied covariate and measure the consequences of its absence in term of prediction quality.

Forward Selection and Backward Selection

Due to the high correlation structure between the covariates, selecting the best model by simply using the k most important variables will not necessary lead to the most accurate prediction. In order to overcome this problem, a classical approach (Diaz 2006; Nguyen, 2013) is to perform a backward selection procedure using random forest.

In case of the forward selection, the idea is to start with the empty model, to perform a sensitivity analysis (one RF by variable), adding the variable providing the lowest improvement of the criterion (here R2 was used, see description below) of interest and start again with the augmented model. Because of the highly stochastic nature of the random forest, the results from a forward selection procedure can vary from a replication to another. Hence, a total of five replications was performed systematically with a high number of trees (tree=50,000) and select a consensus model that appear to be stable across the replications.

In case of the backward selection, the idea is to start with the full model, to perform a sensitivity analysis (one RF by variable), removing the variable providing the lowest improvement of the criterion (here R2 (also designated as r²) was used, see description below) of interest and start again with the reduced model. Because of the highly stochastic nature of the random forest, the results from a backward selection procedure can vary from a replication to another. Hence, a total of five replications was performed systematically with a high number of trees (tree=50,000) and select a consensus model that appear to be stable across the replications.

Linear Regressions

A classical linear regression is also is performed on selected models. Investigating the linear coefficients also provides a simple way to understand the individual effects of the covariates on the response variables.

Leave-One-Out

As the data should not be split into a training and a testing dataset, a classical cross-validation technique was used called “leave-one-out” in order to avoid over-fitting. Using this approach we repetitively leave out of the data one entry, train our model (linear regression or random forest) with the reduced dataset, and then used the resulting model to predict the value of the entry left aside.

R2

The correlation between the response variable and the predicted response is measured in terms of square correlation R2 (or r²). In the linear model context it corresponds exactly to the proportion of explained variance. R2 is always between 0 and 100%, the higher the better.

AUC

The classification performance is measured in term of Area Under the ROC curve (AUC). This classical criterion is often preferred to power since it consider simultaneously all possible thresholds and does not even require to control H0 error rates. AUC is always between 0 and 100%. An AUC around 50% correspond to pure noise, an AUC below 70% is considered weak, an AUC between 70% and 80% is considered correct, between 80% and 90% good, and above 90% excellent. AUC estimation are here performed using the pROC R package (robin2011proc).

Survival Analysis

A standard survival analysis was employed: Cox model (Andersen and Gill, 1982; Therneau, 2000), Kaplan-Meier non parametric hazard estimates (Kaplan and Meier, 1958), and log-rank difference test for significance between survival curves (Harrington and Fleming, 1982). For random forests, the package randomForestSRC was used (Ishwaran and Kogalur, 2007; Ishwaran et al., 2008; Ishwaran and Kogalur, 2015).

3 Results and Discussion

3.1 Fluid Balance

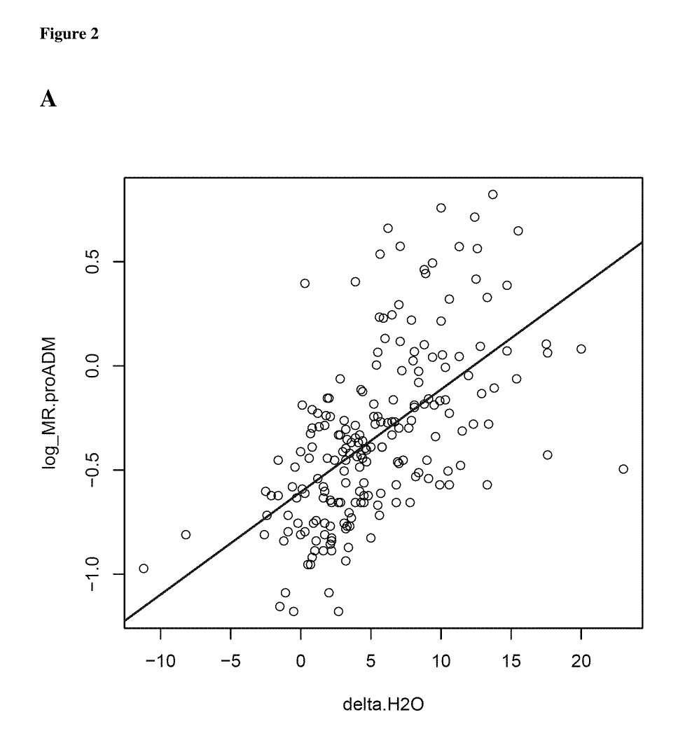

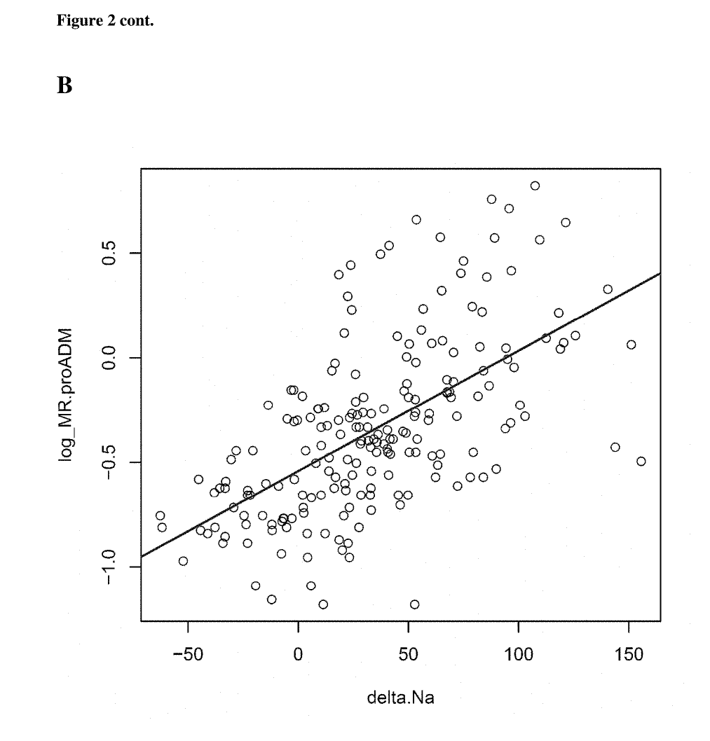

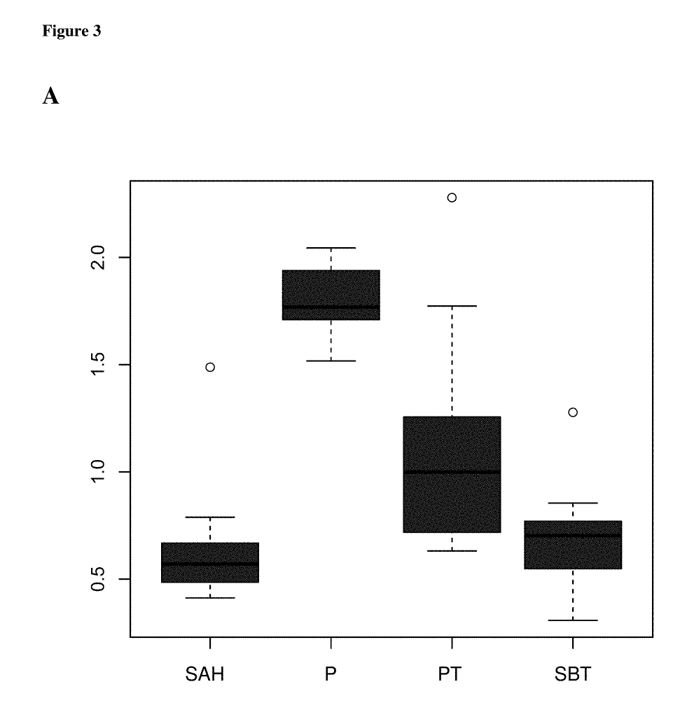

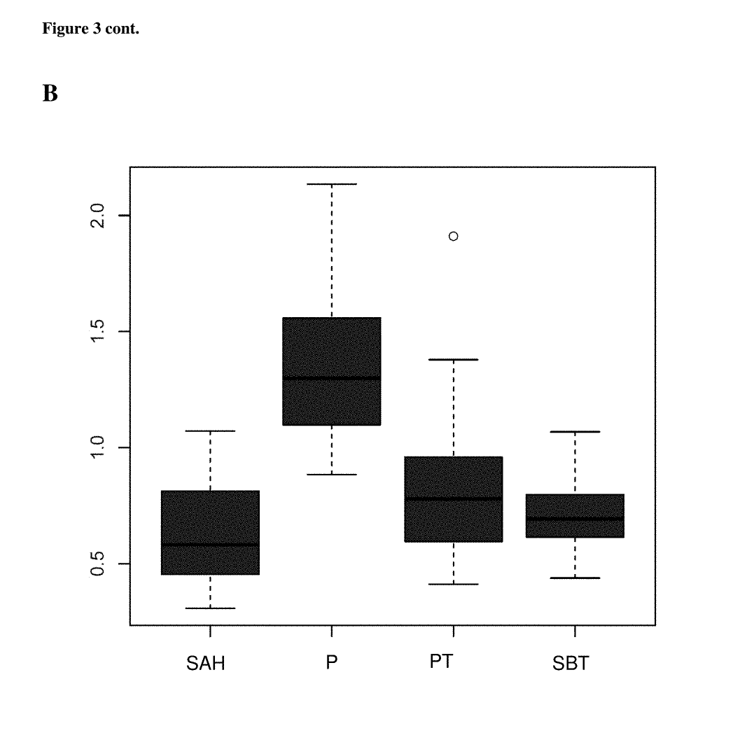

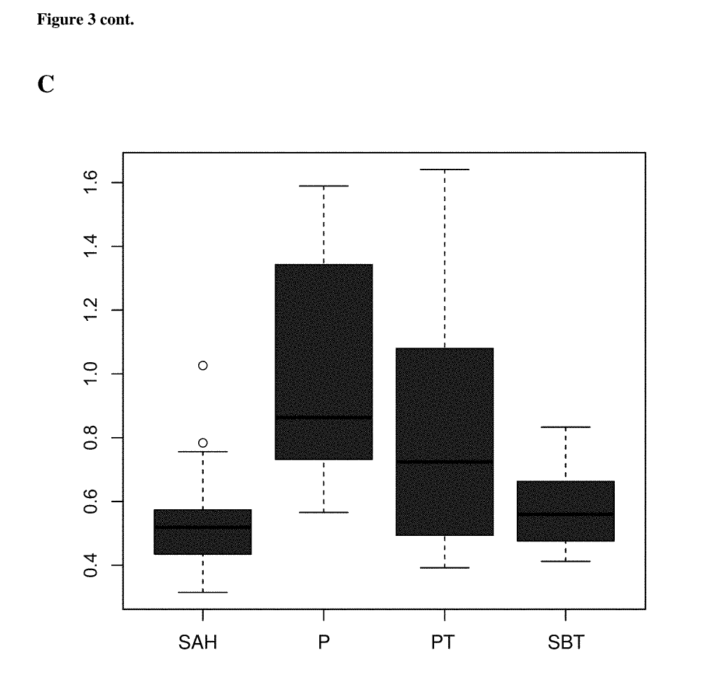

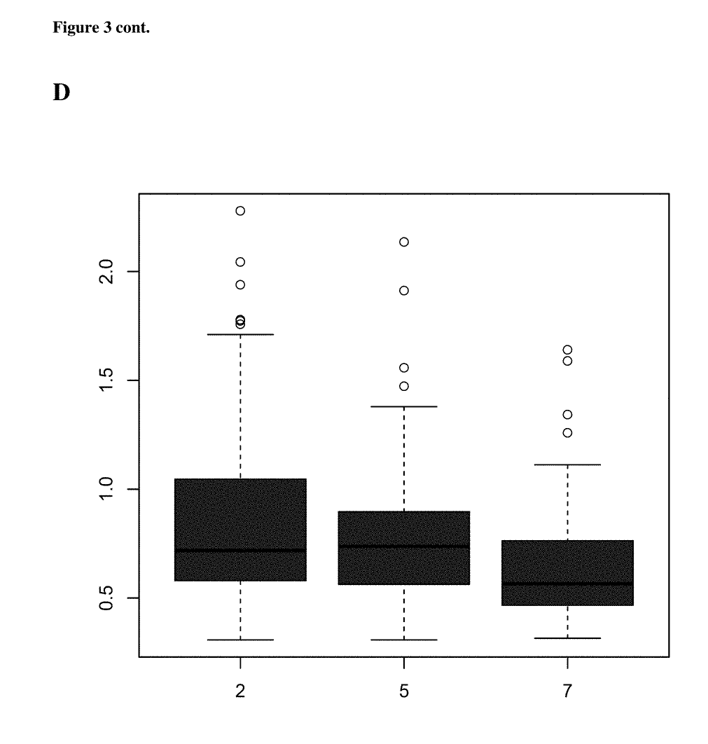

MR.proADM shows a good performance for predicting delta.H2O. MR.proADM by itself achieves a good classification power (AUC=≈0.82) (FIG. 2A) with a response variable of 35% of variance.

The selection procedure for delta.H2O was performed. The importance analysis pointed toward the importance of following patients and daily covariates: bmi.D0, weight.D0, age, sex, BMI, total protein, Hb liquid intake.D2 (fluid.D0), patient group (group). The biomarker, MR.proADM achieved the highest importance. Further biomarkers seem to play a role: Pro.Endo, CT.proAVP, EPO, total serum protein and Hb.

3.2 Salt Balance

As observed for delta.H₂O, MR.proADM has a good classification power (AUC=≈0.79), with r² of 0.42 (FIG. 2B).

The importance analysis performed pointed toward the same markers/parameters as observed for delta.H2O, with the difference that sodium intake at D0 (Na.D0) replaced liquid intake at D0 (Fluid.D0).

4. Conclusions

The key role of MR-proADM for predicting delta.H2O and delta.Na was clearly confirmed.

Nonetheless, a combination of MR-proADM with further makers and/or parameters might improve the prediction of the fluid and/or salt balance.

### EXAMPLE 3

### Improving the Prediction by Including Further Parameters

The objective of the present study is to build clinically exploitable predictors using a selection of covariates (further markers or parameters) (see Table 7).

TABLE 7
Markers and parameters used.

| | |
|---|---|
| Primary | MR.proADM; bmi.J0 (BMI at day 0); weight.J0 (weigth |
| | at D 0); Fluid.J0 (liquid intake at D 0); age; sex |
| Secondary | Pro.Endo (pro-endothelin-1); CT.proAVP; Na.J0 (sodium |
| | intake at day 0); Adre (adrenalin); IGS.II Pro.ANP |
| | FC (heart rate); max.temp (maximal temperature); |
| | min.PAM (minimal of the mid arterial pressure); |
| | max.lactate (lactate) max.cath (catecholamine); |
| | Prot.J0 (total serum protein at day 0); Prot (total serum |
| | protein); Hb; weight |

1.1 Fluid Balance and Sodium Balance

Reference model for predicting delta.H2O:

delta.H2O ˜MR.proADM+bmi.D0+weight.D0+age+sex+Hb+Prot+IGS.II+Fluid.D0

where IGS.II and Fluid.D0 both are optional due to the practical difficulty to obtain them in the clinical context.

TABLE 8
Summary of delta.H2O and delta.Na models.

| response | Model | R2 (lm) | R2 (rf) | sd | CV | AUC | [95% CI] |
|---|---|---|---|---|---|---|---|
| H2O | reference | 0.501 | 0.703 | 2.718 | 0.512 | 0.920 | [0.884-0.956] |
| | no Fluid.D0 | 0.497 | 0.699 | 2.753 | 0.518 | 0.924 | [0.890-0.958] |
| | no IGS.II | 0.504 | 0.699 | 2.742 | 0.516 | 0.921 | [0.886-0.957] |
| | no Fluid.D0/IGS.II | 0.501 | 0.680 | 2.815 | 0.530 | 0.924 | [0.890-0.958] |
| H2O (-D5) | reference | 0.433 | 0.509 | 3.451 | 0.674 | 0.891 | [0.835-0.946] |
| | weight Prot | 0.298 | 0.430 | 3.710 | 0.725 | 0.793 | [0.715-0.871] |
| | weight | 0.251 | 0.194 | 4.575 | 0.894 | 0.712 | [0.624-0.801] |
| | Prot | 0.076 | 0.178 | 4.710 | 0.920 | 0.629 | [0.534-0.724] |
| Na | reference | 0.566 | 0.713 | 23.738 | 0.691 | 0.886 | [0.841-0.931] |
| | no Fluid.D0 | 0.557 | 0.708 | 23.864 | 0.695 | 0.887 | [0.842-0.931] |
| | no IGS.II | 0.569 | 0.704 | 24.001 | 0.699 | 0.880 | [0.834-0.927] |
| | no Fluid.D0/IGS.II | 0.561 | 0.704 | 24.141 | 0.703 | 0.881 | [0.835-0.927] |
| Na (-D5) | reference | 0.553 | 0.541 | 29.571 | 0.876 | 0.851 | [0.786-0.917] |
| | weight Prot | 0.238 | 0.316 | 35.983 | 1.066 | 0.749 | [0.665-0.834] |
| | weight | 0.206 | 0.171 | 40.894 | 1.212 | 0.661 | [0.565-0.756] |
| | Prot | 0.053 | 0.194 | 40.979 | 1.214 | 0.634 | [0.537-0.731] |

As can be seen in Table 8 (response “H2O”) that this model explained roughly 70% of the variance and had a very good discriminative power with an AUC of 92% (lower bound of the 95% CI is roughly 89%). The absence of IGS.II and/or Fluid.D0 had very limited consequences with only a loss of 2-3% of r² and no effect at all on the AUC.

This model was also compared to a predictor built from patient weight and Prot. As weight was not available at D5, this time point was removed completely, thus resulting in a smaller dataset. The results were presented in Table 8 (response “H2O (−D5)”). A predictor built from weight and Prot only achieved an AUC of 80%. Please note that the drop was significant from our reference model (AUC of 90%). Moreover, the loss was even more dramatically when considering only weight (AUC=71%) or Prot (AUC=63%) further proving the limited interest of these two covariates. One should also note that the r² was very limited with these models, especially when using only linear regressions.

Sodium Balance

Reference model for predicting delta.Na:

delta.Na˜MR.proADM+bmi.D0+weight.D0+age+sex+Hb+Prot+Na.D0+IGS.II +Fluid.D0

as observed for delta.H2O, IGS.II and Fluid.D0 are both are optional.

It can be seen in Table 8 (response “Na”) that this model explained roughly 70% of the variance and had a good discriminative power with an AUC of 88% (lower bound of the 95% CI is roughly 84%). As observed for delta.H2O, the absence of IGS.II and/or Fluid.D0 had very limited consequences. Again, as observed for delta.H2O, weight and Prot clearly had a limited interest for predicting the response variables, with both a low R² (˜20%) and AUC (˜63-75%).

Building a Joint Predictor

In this section, a predictor of “critical” patients, i.e. patients who has both delta.H2O >4 L and delta.Na>36 g is developed. For that purpose, the leave-one-out residuals for (delta.H2O, delta.Na) were considered jointly, which a distribution found to be Gaussian (centered) with a covariance matrix:

$$Σ = ( \begin{matrix}7.35 & 33.5 \\ 33.5 & 560\end{matrix} ) .$$

It is therefore possible to compute for each couple (x,y) of predicted values (delta.H2O, delta.Na) the probability to be critical as:

P_{critical}(x, y)= [symbol] [symbol] ((x, y)^{T}, Σ)∈|4.0,=∞|×[36.0,=∞[)

this probability being easily computed through the mvtnorm R package (Genz and Bretz, 2009; Genz et al., 2014).

A graphical representation of the two dimensional Pcritical (p-critical) function would allow to discriminate between non critical and critical patients using only the delta.H2 O and delta.Na predicted values. Note that the discriminative power of this joint predictor is high with AUC=0.92 (see also Example 4).

2.2 Volume Responses

Total Volume and Plasmatic Volume

delta.H2O and delta.Na were surpringly found to be sub-optimal predictors for VT and VP (Table 9). Indeed, they explained at best 7% of the variance, and barely outperform the random classifier in term of discriminative power (best AUC=64%). This clearly demonstrated that the clinical idea that “filling patients” with salted water does not trigger the expected plasmatic response.

TABLE 9
Summary of volume responses models

| re- | | | | | | AUC |
|---|---|---|---|---|---|---|
| sponse | Model | lmR2 | R2 | sd | CV | [95% CI] |
| VT | H2O Na | 0.009 | 0.040 | 12.370 | 0.215 | 0.582 |
| | | | | | | [0.481-0.682] |
| | H2O | 0.007 | 0.037 | 12.896 | 0.224 | 0.579 |
| | | | | | | [0.475-0.684] |
| | Na | 0.017 | 0.069 | 12.533 | 0.218 | 0.636 |
| | | | | | | [0.540-0.733] |
| VP | H2O Na | 0.031 | 0.029 | 9.101 | 0.224 | 0.589 |
| | | | | | | [0.492-0.686] |
| | H2O | 0.095 | 0.021 | 9.674 | 0.239 | 0.571 |
| | | | | | | [0.474-0.669] |
| | Na | 0.017 | 0.037 | 9.458 | 0.233 | 0.573 |
| | | | | | | [0.475-0.671] |
| VG | Ref | 0.408 | 0.455 | 3.263 | 0.192 | 0.824 |
| | | | | | | [0.747-0.900] |
| | no | 0.407 | 0.484 | 3.300 | 0.194 | 0.839 |
| | MR.proADM | | | | | [0.764-0.914] |
| | | 0.304 | 0.230 | 4.052 | 0.239 | 0.698 |
| | only Hb | | | | | [0.593-0.802] |

Globular Volume

For the globular volume (VG), a correlation with delta.H2O and delta.Na was observed. It was possible to build a predicting (reference) model:

VG˜MR.proADM+Hb+bmi.D0+sex+age+Prot

As can be seen in Table 9, this model explained 45% of variance (which is low) but nevertheless provided an AUC of 82% (lower 95% CI bound is 75%). This was clearly an improvement over the simple model using exclusively Hb, which obtained a r² of only 30% (with the linear model rather than the random forests) and an AUC of 70% (lower 95% bound is 60%). This result was interesting as it emphasized the limits of Hb as a single biomarker for the globular volume response, which is the current clinical standard.

It can be seen in Table 9 that the “reference” model without MR-proADM achieved a similar performance with a r² of 48% and AUC of 84%. The reference model including MR-proADM identifies patients with low (<20 or 15) VG or RBCV.

When considering the leave-one-out residuals of the best VG models, we clearly observed that these residuals are distributed according to a Gaussian centered distribution with a standard deviation of 3.2-3.3. Despite the fact that a large proportion of the observed variance was not explained by this model, one can already use them to detect critical patients: patients with VG<20 mL/kg. Indeed, if x is the predicted VG, we have:

P_{critical}(x)= [symbol] [symbol] (x.3.3²) <20)=dnorm(20, mean=x, sd=3.3).

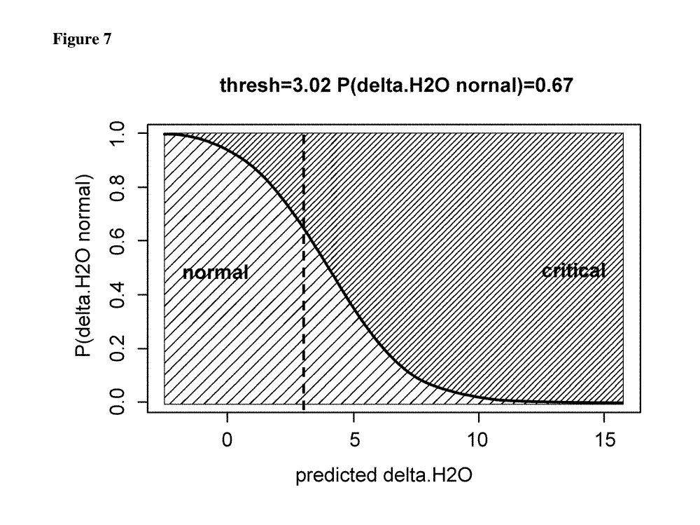

FIG. 7 illustrates the Pcritical function. This decision function might provide a valid alternative to Hb to detect critical patients in a reliable way.

2.3 SOFA

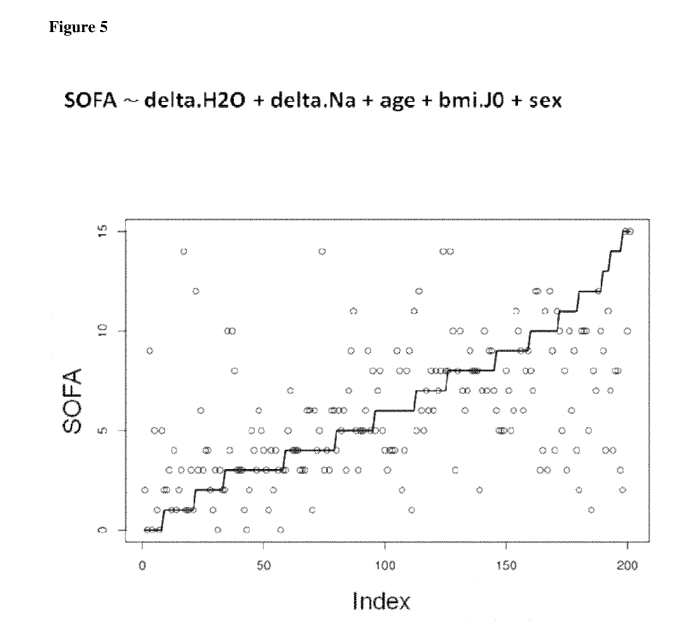

In FIG. 5, the predicted SOFA values using the model selected by the experts and a leave-one-out procedure are illustrated. The correlation was very high, even if the prediction accuracy was not huge. Indeed, in Table 10, exact predictions (difference=0) had only 19% accuracy, but this number dramatically increased in case more differences between predicted and exact SOFA were allowed. For a difference of 4, a SOFA of 82% was obtained.

SOFA˜delta.H2O+delta.Na+age+bmi.D0+sex

TABLE 10
SOFA prediction accuracy

| difference | 0 | 1 | 2 | 3 | 4 |
|---|---|---|---|---|---|
| accuracy | 0.19 | 0.47 | 0.69 | 0.77 | 0.82 |

Note that we also tried to regroup SOFA into three classes with no significant improvement (33% error rate). 2.4 Edema Duration

We can see in Table 11 the available data.

TABLE 11
Edema duration data. A total of 21 observed endpoints (no more edema) and 31
censored duration.

| | time | | | | | | | | | | | | | | | | | |
|---|---|---|---|---|---|---|---|---|---|---|---|---|---|---|---|---|---|---|
| | 8 | 5 | 7 | 7 | 4 | 7 | 7 | 7 | 8 | 6 | 8 | 8 | 4 | 8 | 5 | 10 | 8 | 9 |
| delta | 0 | 1 | 1 | 0 | 1 | 1 | 0 | 1 | 0 | 0 | 0 | 0 | 1 | 0 | 1 | 0 | 0 | 0 |
| | time | | | | | | | | | | | | | | | | | |
| | 3 | 6 | 8 | 6 | 6 | 2 | 6 | 8 | 7 | 6 | 6 | 9 | 4 | 7 | 4 | 3 | 7 | 8 |
| delta | 1 | 1 | 0 | 1 | 0 | 1 | 0 | 0 | 0 | 1 | 1 | 0 | 1 | 0 | 1 | 1 | 0 | 0 |

| | time | | | | | | | | | | | | | | | |
|---|---|---|---|---|---|---|---|---|---|---|---|---|---|---|---|---|
| | 7 | 7 | 6 | 6 | 7 | 6 | 6 | 6 | 8 | 8 | 9 | 5 | 10 | 9 | 7 | 7 |
| delta | 0 | 0 | 1 | 0 | 0 | 1 | 1 | 0 | 0 | 0 | 0 | 1 | 1 | 0 | 0 | 0 |

TABLE 12
Cox model for edema duration. Significant
covariates are marked with a star.

| | coef | exp(coef) | se(coef) | z | p |
|---|---|---|---|---|---|
| age | −0.05 | 0.95 | 0.02 | −2.54 | 0.01* |
| sexM | 0.94 | 2.55 | 0.93 | 1.01 | 0.31 |
| weight.D 0 | −0.07 | 0.93 | 0.05 | −1.62 | 0.11 |
| bmi.D 0 | 0.14 | 1.14 | 0.13 | 1.03 | 0.30 |
| IGS.II | 0.00 | 1.00 | 0.02 | 0.13 | 0.90 |
| SOFA | −0.21 | 0.81 | 0.10 | −2.13 | 0.03* |
| MR.proADM | 0.64 | 1.90 | 0.88 | 0.73 | 0.46 |

Cox Model

The analysis was started by fitting a proportional hazard model on these data (Table 12). Among the tested covariates, only age and SOFA were significant. In particular, MR.proADM did not appear to play a key role. We built adjusted edema duration by fitting a Cox model with only age and SOFA, with the reference being 41.5 for the age (median) and 9 for the SOFA (median).

Kaplan-Meier Estimates

Kaplan-Meier estimates of the survival curves with a stratification on low (MR.proADM<1.5) and high (MR.proADM>1.5) values at D2 were analyzed. The difference between the two curves was clearly not significant, which confirmed the results of Table 12. Note that when using the unadjusted edema duration, a significant difference (p=0.01) between the two curves were observed (data not shown), but this result vanished when adjusting on age and SOFA.

Random Forests

On the same (unadjusted) data random forests were performed. The procedure achieved a global error rate of 41% which is high (exact nature of this error rate was unclear). In terms of variable importance, the key role of age was confirmed, but SOFA and MR.proADM both appeared to have a weak influence on the result of this non linear framework. Using leave-one-out technique, the random forests were used to predict survival curves for each patient based on the covariates (age, SOFA and MR.proADM). The difference between the two data sets was mainly due to the fact that patients with high MR-proADM at D2 were mostly not healed at the end of the study. However, patients with lower MR-proADM at D2 had no clear trend towards healing, once the effects of age and SOFA were included.

3. Conclusions

For globular volume, MR.proADM had a reasonable relevance. Moreover, our study points out the limits of the current biomarker (Hb) and suggests a new model that might be useful for the clinicians in the future for monitoring patients with critical VG.

The more interesting achievement of this study clearly is the predictor of edema which combines delta.H2O and delta.Na prediction to detect very efficiently (AUC>0.99) patients with critical edema risk using only easy to obtain clinical covariates (bmi.D0, weight.D0, age, sex and optionally IGS.II and Fluid.D0) with three biomarkers: MR.proADM, Hb, and Prot.

### EXAMPLE 4

### Further Improving the Prediction

Models

In order simplify the models provided herein, IGS.II, Fluid.J0 and Na.J0 were removed from the original models in order to get a single simple model both for predicting delta.H2O and delta.Na (in the following designated as “model 2”). The inclusion of age2=age² and age3=age³ to the model further improved the predictive power. This was the only addition of transformed covariates which appeared to have significant effect. The models presented in Example 3 for predicting the fluid balance and salt balance, i.e. including the parameters IGS.II, Fluid.J0 and Na.J0, are designated as “model 1”.
“model 2”: bmi.J0+weight.J0+age+age2+age3+sex+MR.proADM+Hb+Prot
“without biomarkers”: bmi.J0+weight.J0+age+age2+age3+sex
“only biomarkers”: MR.proADM+Hb+Prot

NB: Please note that MR.proADM, Hb and Prot refer to log 1p transform of the original measurements.

For each of these models, we can either perform prediction using the leave-one-out approach or using the complete set of data. Unsurprisingly, performances will always be higher in the latter case than in the former. For robust and possible replicable results, one should prefer the leave-one-out estimations, for a more optimistic point of view, as well as for comparing with very crude methods (like using directly MR.proADM to discriminate between regular and critical patients), one should use the complete dataset estimations.

AUC Results

TABLE 13
AUC in the leave-one-out framework.

| AUC | | | |
|---|---|---|---|
| [95% CI] | delta.H2O | delta.Na | Pcritical |
| model 2 | 0.923 | 0.917 | 0.926 |
| | [0.887-0.959] | [0.881-0.953] | [0.892-0.960] |
| model 1 | 0.922 | 0.919 | 0.922 |
| | [0.886-0.959] | [0.883-0.955] | [0.888-0.957] |
| no | 0.823 | 0.815 | 0.825 |
| biomarkers | [0.763-0.882] | [0.751-0.878] | [0.764-0.886] |
| only | 0.884 | 0.881 | 0.886 |
| biomarkers | [0.840-0.929] | [0.835-0.927] | [0.841-0.930] |

TABLE 14
AUC using all the available data

| | delta.H2O | delta.Na | Pcritical |
|---|---|---|---|
| model 2 | 0.948 | 0.981 | 0.990 |
| | [0.922-0.974] | [0.967-0.996] | [0.981-0.999] |
| model 1 | 0.947 | 0.983 | 0.990 |
| | [0.921-0.974] | [0.968-0.997] | [0.981-0.999] |
| no | 0.879 | 0.902 | 0.911 |
| biomarkers | [0.832-0.926] | [0.857-0.946] | [0.870-0.952] |
| only | 0.942 | 0.976 | 0.977 |
| biomarkers | [0.913-0.970] | [0.959-0.993] | [0.961-0.993] |

The herein provided model 2 using only simple covariates and selected biomarkers achieved similar or even better performance than the model 1 presented in Example 3. In all situations, Pcritical appeared to combine efficiently delta.H2O and delta.Na prediction with a slight improvement over the best of the two methods. When considering the model without any biomarker, there was a significant drop of performance One should however note that this model nevertheless points out the high edema risk patients. When considering only biomarkers, the performance was similar compared to the best model, but it was still inferior.

For comparison purpose, the performance of MR.proADM alone to distinguish between regular and critical patients achieved AUC=0.845 [0.791-0.898] which must be compared to the AUC of Table 14 (0.990 for the best model) in order to be consistent. Therefore, the combination of further markers and/or parameters provided even a further improvement of the predicitive power.

Details on the Best Model

The ROC obtained with the three different methods was compared, i.e., 1) using only predicted delta.H2O; 2) using only predicted delta.Na; 3) combining both predictions into Pcritical. If delta.H2O is much less efficient than the other two (which is consistent with Table. 13 and Table 14), ROC for delta.Na and Pcritical are very similar. However, Pcritical is superior than delta.Na for high specificity (ex: Spe >0.95). This can be highlighted by considering (adjusted) partial AUC for Spe∈[1.00,0.951]. The value of 0.781 for delta.H2O, the value of 0.882 for delta.Na, and the value of 0.948 for Pcritical was obtained. These results suggested that Pcritical was even more reliable than delta.Na (and delta.H2O) when a high Specificity is required.

TABLE 15
False Positive (FP), False Negative (FN), Sensitivity
(Sen) and Specificity (Spe) for the best model (all
data) for various threshold levels on Pcritical.

| threshold | FP | FN | Sen | Spe |
|---|---|---|---|---|
| 0.10 | 40 | 0 | 1.00 | 0.68 |
| 0.20 | 28 | 0 | 1.00 | 0.78 |
| 0.30 | 23 | 1 | 0.99 | 0.82 |
| 0.40 | 15 | 3 | 0.96 | 0.88 |
| 0.50 | 10 | 4 | 0.95 | 0.92 |
| 0.60 | 4 | 6 | 0.92 | 0.97 |
| 0.70 | 1 | 9 | 0.88 | 0.99 |
| 0.80 | 1 | 10 | 0.87 | 0.99 |
| 0.90 | 0 | 20 | 0.73 | 1.00 |

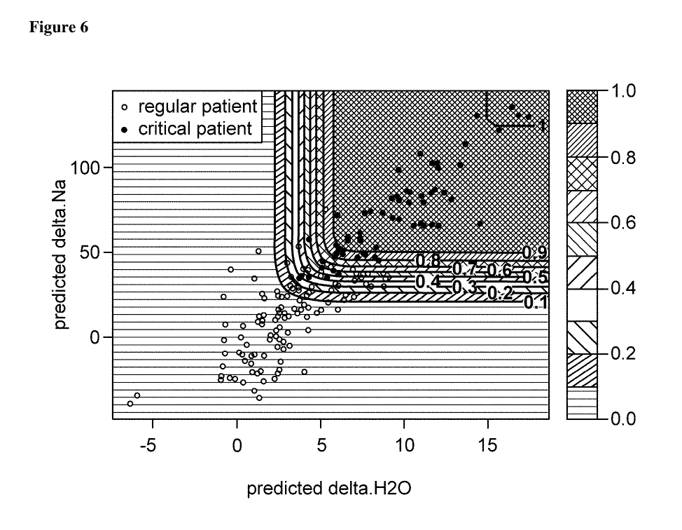

In Table 15, the performance of the prediction using Pcritical with various threshold was summarized. Depending on the cost of False Positive and False Negative, this should allow to choose the threshold achieving the best compromise between the two concurrent risks. The predicted delta.H2O to the predicted delta.Na for 201 patients included in the study was analyzed (FIG. 6). Both predictions are highly correlated (cor≈0.9), which is consistent with the observed delta.H2O and delta.Na (cor≈0.8). The representation of Pcritical demonstrated that high risk regions were represented almost exclusively by “critical” patients, and low risk regions almost exclusively by “regular” ones.

Correlation between Pcritical, SOFA and VG

In this section, we do compare the new Pcritical score to SOFA and VG (Globular Volume). We can see in Table 16 and Table 17 that there is a good correlation between SOFA and Pcritical.

From now on, we focus on two particular groups of interest: the “low risk” group gathering a total of 86 patients (out of 201) with Pcritical <0:1, and the “high risk” group gathering 55 patients with Pcritical >0:9.

TABLE 16
SOFA repartition by groups of Pcritical.

| SOFA | 0 | 1 | 2 | 3 | 4 | 5 | 6 | 7 | 8 | 9 | 10 | 11 | 12 | 13 | 14 | 15 | 17 | 20 | 21 |
|---|---|---|---|---|---|---|---|---|---|---|---|---|---|---|---|---|---|---|---|
| 0.00 < P_{critical} < 1.00 | 8 | 13 | 12 | 25 | 21 | 16 | 17 | 13 | 20 | 14 | 12 | 8 | 10 | 3 | 5 | 1 | 1 | 1 | 1 |
| 0.00 < P_{critical} < 0.25 | 6 | 11 | 11 | 15 | 14 | 10 | 7 | 2 | 6 | 8 | 6 | 3 | 2 | 1 | 0 | 0 | 0 | 0 | 0 |
| 0.25 < P_{critical} < 0.50 | 1 | 0 | 0 | 3 | 1 | 1 | 4 | 3 | 1 | 0 | 1 | 1 | 1 | 0 | 0 | 1 | 0 | 0 | 0 |
| 0.50 < P_{critical} < 0.75 | 1 | 0 | 0 | 3 | 2 | 2 | 0 | 1 | 0 | 1 | 2 | 1 | 1 | 0 | 0 | 0 | 0 | 0 | 0 |
| 0.75 < P_{critical} < 1.00 | 0 | 2 | 1 | 3 | 4 | 2 | 6 | 7 | 13 | 4 | 3 | 3 | 6 | 2 | 5 | 0 | 1 | 1 | 1 |

TABLE 17
SOFA distribution by groups of Pcritical

| selection | Min. | 1st Qu. | Median | Mean | 3rd Qu. | Max. |
|---|---|---|---|---|---|---|
| 0.00 < P_{critical} < 1.00 | 0.00 | 3.00 | 6.00 | 6.29 | 9.00 | 21.00 |
| 0.00 < P_{critical} < 0.25 | 0.00 | 2.00 | 4.00 | 4.80 | 7.75 | 13.00 |
| 0.25 < P_{critical} < 0.50 | 0.00 | 4.25 | 6.00 | 6.61 | 7.75 | 15.00 |
| 0.50 < P_{critical} < 0.75 | 0.00 | 3.25 | 5.00 | 6.14 | 9.75 | 12.00 |
| 0.75 < P_{critical} < 1.00 | 1.00 | 6.00 | 8.00 | 8.64 | 11.25 | 21.00 |

It is confirmed that SOFA was significantly higher in the “high risk” group than in the “low risk” group. In contrast, VG was significantly lower in the “high risk” group. Finally, Pcritical was used to discriminate between patients with VG<15 (91 cases) and patients with VG≥15 (43 controls). An AUROC of 0:76 (95% CI is [0.67-0.85]) was obtained. Note that the CI is quite large due to the fact that we have only 134 measurements of VG in the dataset.

Conclusions

The model provided herein using only biomarkers (MR.proADM, Hb, Prot) and simple covariates (bmi, weight, age, sex) achieved a maximum AUC of 0.926 in the leave-one-out framework, and an AUC of 0.990 when using all data. This performance is excellent. In terms of clinical application, charts as provided in FIG. 6 could provide useful information to the clinician.

All references cited herein are fully incorporated by reference. Having now fully described the invention, it will be understood by a person skilled in the art that the invention may be practiced within a wide and equivalent range of conditions, parameters and the like, without affecting the spirit or scope of the invention or any embodiment thereof.

Rivers E, Nguyen B, Haystad S, Ressler J, Muzzin A, Knoblich B, Peterson E, Tomlanovich M. Early Goal-Directed Therapy Collaborative Group: Early goal-directed therapy in the treatment of severe sepsis and septic shock. N Engl J Med 2001, 345:1368-1377.

Chappell D, Jacob M, Klaus Hofmann-Kiefer K, Conze P, Rehm M. A Rational Approach to Perioperative Fluid Management, Anesthesiology 2008; 109:723-40

Sakr Y, Vincent J L, Reinhart K, Groeneveld J, Michalopoulos A, Sprung C L, Artigas A, Ranieri V M; Sepsis Occurence in Acutely Ill Patients Investigators. High tidal volume and positive fluid balance are associated with worse outcome in acute lung injury. Chest. 2005; 128(5): 3098-108

Bagshaw S M, Brophy P D, Cruz D, Ronco C. Fluid balance as a biomarker: impact of fluid overload on outcome in critically ill patients with acute kidney injury. Crit Care. 2008; 12(4) :169.

Payen D, de Pont A C, Sakr Y, Spies C, Reinhart K, Vincent J L; Sepsis Occurrence in Acutely Ill Patients (SOAP) InvestigatorsA positive fluid balance is associated with a worse outcome in patients with acute renal failure. Crit Care. 2008; 12(3):R74.

Murphy C V, Schramm G E, Doherty J A, Reichley R M, Gajic O, Afessa B, Micek S T, Kollef M H. The importance of fluid management in acute lung injury secondary to septic shock. Chest. 2009; 136(1):102-9.

Boyd J H, Forbes J, Nakada T A, Walley K R, Russell J A. Fluid resuscitation in septic shock: a positive fluid balance and elevated central venous pressure are associated with increased mortality. Crit Care Med. 2011; 39(2):259-65.

Kelm D J, Perrin J T, Cartin-Ceba R, Gajic O, Schenck L, Kennedy C C. Fluid overload in patients with severe sepsis and septic shock treated with early goal-directed therapy is associated with increased acute need for fluid-related medical interventions and hospital death. Shock. 2015; 43(1):68-73.

Acheampong A, Vincent J L. A positive fluid balance is an independent prognostic factor in patients with sepsis. Crit Care. 2015; 19(1):251.

Cecconi M, Parsons A K, Rhodes A. What is a fluid challenge? Cur Opin Crit Care 2011 17:290-295.

Chappell D, Westphal M, Jacob M. The impact of the glycocalyx on microcirculatory oxygen distribution in critical illness. Curr Opin Anaesthesiol. 2009; 22(2):155-62. Review

Jacob M, Chappell D, Hollmann M W. Current aspects of perioperative fluid handling in vascular surgery. Curr Opin Anaesthesiol. 2009; 22(1):100-8. Review.

Ostrowski S R, Haase N, Muller R B, Moller M H, Pott F C, Perner A, Johansson P I. Association between biomarkers of endothelial injury and hypocoagulability in patients with severe sepsis: a prospective study. Crit Care. 2015 Apr 24; 19:191.

Sakr Y, Lobo S M, Moreno R P, Gerlach H, Ranieri V M, Michalopoulos A, Vincent J L; SOAP Investigators. Patterns and early evolution of organ failure in the intensive care unit and their relation to outcome. Crit Care. 2012; 16(6):R222.

Besen B A, Gobatto A L, Melro L M, Maciel A T, Park M. Fluid and electrolyte overload in critically ill patients: An overview. World J Crit Care Med. 2015; 4(2):116-29.

Lobo D N, Bostock K A, Neal K R, Perkins A C, Rowlands B J, Allison S P. Effect of salt and water balance on recovery of gastrointestinal function after elective colonic resection: a randomised controlled trial. Lancet. 2002; 359(9320):1812-8.

Brandstrup B, Tønnesen H, Beier-Holgersen R, Hjortsø E, er al. Danish Study Group on Perioperative Fluid Therapy. Effects of intravenous fluid restriction on postoperative complications: comparison of two peri-operative fluid regimens: a randomized assessor-blinded multicenter trial. Ann Surg 2003, 238:641-648.

Bjerregaard L S, Mø11er-Sørensen H, Hansen K L, Ravn J, Nilsson J C. Using clinical parameters to guide fluid therapy in high-risk thoracic surgery. A retrospective, observational study.Walsh SR, Walsh CJ. Intravenous fluid-associated morbidity in postoperative patients. Ann R Coll Surg Engl. 2005; 87(2):126-30.

de Kruif M D, Lemaire L C, Giebelen I A, Struck J, Morgenthaler N G, Papassotiriou J, Elliott P J, van der Poll T. The influence of corticosteroids on the release of novel biomarkers in human endotoxemia. Intensive Care Med. 2008; 34(3):518-22.

Schweda F. Salt feedback on the renin-angiotensin-aldosterone system. Pfiugers Arch. 2015; 467(3):565-76.

Farag E; Maheshwari K, Morgan J, Sakr E, Wael A, Doyle J. An Update of the Role of Renin Angiotensin in Cardiovascular Homeostasis. Anesth Analg. 2015; 120(2): 275-292

Struck J, Morgenthaler N G, Bergmann A. Copeptin, a stable peptide derived from the vasopressin precursor, is elevated in serum of sepsis patients, Peptides 26 (2005) 2500-2504 Morgenthaler N G, Muller B, Struck J, Bergmann A, Redl H Christ-Crain M. Copeptin, a stable peptide of the arginine vasopressin precursor is elevated in hemorrhagic and septic shock. 2007. 28: 219-226

Zweifel C, Katan M, Schuetz P et al. Copeptin is associated with mortality and outcome in patients with acure intracerebral hemorrhage. BMC Neurol. 2010; 10:34.

Urwyler S A, Schuetz P, Fluri F et al. Pronostic value of copeptin. One-year outcome patienty with acute stroke. Stroke 41:1564-1567, 2010.

Kohan D E, Rossi N F, Inscho E, Pollock D M. Regulation of blood pressure and salt homeostasis by endothelin. Physiol Rev 91: 1-77, 2011

Breymann C, Rohling R, Huch A, Huch R. Intraoperative endogenous erythropoietin levels and changes in intravascular blood volume in healthy humans. Ann Hematol 2000; 79:183-186

Christ-Crain M., Morgenthaler N G, Struck J, Harbarth S, Bergmann A, Muller B. Mid-regional pro-adrenomedullin as a prognostic marker in sepsis: an observational study. Crit Care 2005; 9:R816-R824.

Koyama T, Ochoa-Callejero L, Sakurai T, et al. Vascular endothelial adrenomedullin-RAMP2 system is essential for vascular integrity and organ homeostasis. Circulation. 2013 127(7):842-53.

Brisman J L, Song J K, Newell D W. Cerebral aneurysms. N Engl J Med. 2006; 355(9): 928-39.

Vincent J L, Moreno R, Takala J, Willatts S, De Mendonça A, Bruining H, Reinhart C K, Suter P M, Thijs L G: The SOFA Sepsis-related Organ Failure Assessment. score to describe organ dysfunction/failure. On behalf of the Working Group on Sepsis-Related Problems of the European Society of Intensive Care Medicine. Intensive Care Med 1996, 22: 707-710.

Feissel M, Michard F, Faller J P, Teboul J L. The respiratory variation in inferior vena cava diameter as a guide to fluid therapy. Intensive Care Med. 2004; 30(9):1834-7.

Feissel M, Michard F, Mangin I, Ruyer 0, Faller J P, Teboul J L. Respiratory changes in aortic blood velocity as an indicator of fluid responsiveness in ventilated patients with septic shock. Chest. 2001; 119(3):867-73.

Monnet X, Teboul L. Assessment of volume responsiveness during mechanical ventilation: recent advances. Crit Care. 2013; 17(2):217.

Gore C J, Hopkins W G, Burge C M. Errors of measurement for blood volume parameters: a meta-analysis. J Appl Physiol 2005; 99:1745-1758.

Fairbanks V F, Klee G G, Wiseman G A, Hoyer J D, Tefferi A, Petitt R M, Silverstein M N. Measurement of blood volume and red cell mass: re-examination of 51Cr and 1251 methods. Blood Cells Mol Dis. 1996; 22(2):169-86.

Dorhout Mees S M, Hoff R G, Rinkel G J E, Algra A, van den Bergh W M. Brain natuiretic peptide concentrations after aneuvrismal subarachnoid hemorrhage: relationship with hypovolemia and hyponatremia. Neurocrit care, 2011

Perrino A C Jr, Harris S N, Luther M A. Intraoperative determination of cardiac output using multiplane transesophageal echocardiography: a comparison to thermodilution. Anesthesiology 1998; 89:350-7

Genz A, Bretz F. Computation of Multivariate Normal and t Probabilities. Lecture Notes in Statistics. Springer-Verlag, Heidelberg, 2009. ISBN 978-3-642-01688-2.

Genz A, Bretz F, Miwa T, Mi X, et al. mvtnorm: Multivariate Normal and t Distributions, 2014. URL http://CRAN.R-projectorg/package=mvtnorm. R package. version 1.0-2.

Malbrain M L, Marik P E, Witters I, Cordemans C, Kirkpatrick A W, Roberts D J, Van Regenmortel N. Fluid overload, de-resuscitation, and outcomes in critically ill or injured patients: a systematic review with suggestions for clinical practice. Anaesthesiol Intensive Ther. 2014; 46(5):361-80.

Vincent J L, Rhodes A, Perel A, Martin G, Rocca G, Vallet B et al Update on hemodynamic monitoring: a consensus of 16. Critical Care 2011, 15:229 (18 August 2011)

Titze J. Sodium balance is not just a renal affair. Curr Opin Nephrol Hypertens. 2014; 23(2):101-5.

Jantsch J, Binger K J, Muller D N, Titze J. Macrophages in homeostatic immune function. Front Physiol. 2014 May 5; 5:146.

Kahn M L. Blood is thicker than lymph. J Clin Invest. 2008; 118(1):23-6.

Bark B P, Persson J, Grande P O. Importance of the infusion rate for the plasma expanding effect of 5% albumin, 6% HES 130/0.4, 4% gelatin, and 0.9% NaCl in the septic rat. Crit Care Med. 2013; 41(3):857-66.

Takanishi D M, Yu M, Lurie F, Biuk-Aghai E, Yamauchi H, Ho H C, Chapital A D. Peripheral blood hematocrit in critically ill surgical patients: an imprecise surrogate of true red blood cell volume. Anesth Analg. 2008; 106(6):1808-12

Naidech A M, Jovanovic B, Wartenberg K E, Parra A, Ostapkovich N, Connolly E S, Mayer S A, Commichau C. Higher hemoglobin is associated with improved outcome after subarachnoid hemorrhage. Crit Care Med. 2007; 35(10):2383-9

K. Kitamura et al.: Cloning And Characterization of cDNA Encoding a Precursor for Human Adrenomedullin, Biochem. Biophys. Res. Commun. 194: 720-725 (1993)

Per Kragh Andersen and Richard D Gill. Cox's regression model for counting processes: a large sample study. The annals of statistics, pages 1100-1120, 1982.

Anne-Laure Boulesteix, Silke Janitza, Jochen Kruppa, and Inke R Konig. Overview of random forest methodology and practical guidance with emphasis on computational biology and bioinformatics. Wiley Interdisciplinary Reviews: Data Mining and Knowledge Discovery, 2 (6):493-507, 2012.

Leo Breiman. Random forests. Machine learning, 45 (1): 5-32, 2001.

Leo Breiman. Manual on setting up, using, and understanding random forests v3. 1. Statistics Department University of California Berkeley, Calif., USA, 2002.

Ramón Díaz-Uriarte and Sara Alvarez De Andres. Gene selection and classification of microarray data using random forest. BMC bioinformatics, 7 (1): 3, 2006.

David P Harrington and Thomas R Fleming. A class of rank test procedures for censored survival data. Biometrika, 69 (3): 553-566, 1982.

H. Ishwaran and U.B. Kogalur. Random survival forests for r. R News, 7 (2): 25-31, October 2007. URL http://CRAN.R-project.org/doc/Rnews/.

H. Ishwaran and U. B. Kogalur. Random Forests for Survival, Regression and Classification(RF-SRC), 2015. URL http://cran.r-project.org/web/packages/randomForestSRC/. R package version 1.6.1.

H. Ishwaran, U. B. Kogalur, E. H. Blackstone, and M. S. Lauer. Random survival forests. Ann. Appl. Statist., 2 (3): 841-860, 2008. URL http://arXiv.org/abs/0811.1645v1.

Edward L Kaplan and Paul Meier. Nonparametric estimation from incomplete observations. Journal of the American statistical association, 53 (282): 457-481, 1958.

Cuong Nguyen, Yong Wang, and Ha Nam Nguyen. Random forest classifier combined with feature selection for breast cancer diagnosis and prognostic. Journal of Biomedical Science and Engineering, 6 (5): 31887, 2013.

R Core Team. R: A Language and Environment for Statistical Computing. R Foundation for Statistical Computing, Vienna, Austria, 2013. URL http://www.R-project.org/.

Xavier Robin, Natacha Turck, Alexandre Hainard, Natalia Tiberti, Frédérique Lisacek, Jean-Charles Sanchez, and Markus Müller. pROC: an open-source package for R and S+ to analyze and compare ROC curves. BMC bioinformatics, 12 (1): 77, 2011.

Terry M Therneau. Modeling survival data: extending the Cox model. Springer, 2000.

Jacob M, Annaheim S, Boutellier U, Hinske C, Rehm M, Breymann C, Krafft A. Haematocrit is invalid for estimating red cell volume: a prospective study in male volunteers. Blood Transfus. 2012 Oct;10(4):471-9. doi: 10.2450/2012.0111-11. Epub 2012 May 4.

SAFE Study Investigators, Finfer S, McEvoy 5, Bellomo R, McArthur C, Myburgh J, Norton R. Impact of albumin compared to saline on organ function and mortality of patients with severe sepsis. Intensive Care Med. 2011 Jan; 37(1):86-96. doi: 10.1007/s00134-010-2039-6. Epub 2010 Oct 6.

Jozwiak M, Silva S, Persichini R, Anguel N, Osman D, Richard C, Teboul J L, Monnet X. Extravascular lung water is an independent prognostic factor in patients with acute respiratory distress syndrome. Crit Care Med. 2013 Feb; 41(2):472-80. doi: 10.1097/CCM.0b013e31826ab377.

Elofson K A, Eiferman D A, Porter K, Murphy C V. Impact of late fluid balance on clinical outcomes in the critically ill surgical and trauma population. J Crit Care. 2015 Jul 16. pii: S0883-9441(15)00376-7. doi: 10.1016/j.jcrc.2015.07.009. [Epub ahead of print] PMID: 26341457

Kissoon N R, Mandrekar J N, Fugate J E, Lanzino G, Wijdicks E F, Rabinstein A A. Positive Fluid Balance Is Associated With Poor Outcomes in Subarachnoid Hemorrhage. J Stroke Cerebrovasc Dis. 2015 Oct; 24(10):2245-51. doi: 10.1016/j.jstrokecerebrovasdis.2015.05.027. Epub 2015 Aug. 13.

Vincent J L, Moreno R, Takala J, Willatts S, De Mendonca A, Bruining H, Reinhart C K, Suter P M, Thijs L G. The SOFA (Sepsis-related Organ Failure Assessment) score to describe organ dysfunction/failure. On behalf of the Working Group on Sepsis-Related Problems of the European Society of Intensive Care Medicine. Intensive Care Med. 1996 Jul; 22(7):707-10.

Klein H G, Flegel W A, Natanson C. Red Blood Cell Transfusion: Precision vs Imprecision Medicine. JAMA. 2015 Oct 20; 314(15):1557-8. doi: 10.1001/jama.2015.10890.

1. A method for determining the extracellular volume status, the fluid balance, the salt balance and/or the globular volume status of a subject, wherein the method comprises determining in a sample obtained from said subject the level of proadrenomedullin (proADM) or a fragment thereof, optionally the fragment is MR-proADM.

2. The method of claim 1, wherein the method comprises:
(a1) comparing said level of proADM or a fragment thereof, optionally MR-proADM, to a reference level of proADM or said fragment thereof, optionally MR-proADM, of at least one reference subject or a population of reference subjects; or
(a2) comparing said level of proADM or said fragment thereof, optionally MR-proADM, to the level of proADM or said fragment thereof, optionally MR-proADM, of the same subject obtained from prior analysis; and
(b) identifying the extracellular volume status, the globular volume status, the fluid balance and/or the salt balance of said subject based on the comparison in (a1) or (a2), respectively.

3. The method of claim 2, wherein the reference subjects are healthy subjects.

4. The method of claim 3, wherein
(i) an increased level of proADM or said fragment thereof, optionally MR-proADM, of the subject as compared to said reference level indicates that said subject has a positive fluid balance, a positive salt balance, a critical globular volume status and/or a critical extracellular volume status;
(ii) an identical or similar level of proADM or said fragment thereof, optionally MR-proADM, of the subject as compared to said reference level indicates that said subject has an identical or similar fluid balance, and/or an identical or similar salt balance;
wherein said identical fluid balance and/or salt balance indicates that the subject has a normal extracellular volume status and/or a normal globular volume status; and/or
(iii) a decreased level of proADM or said fragment thereof, optionally MR-proADM, of the subject as compared to the reference level indicates that said subject has a negative fluid balance and/or a negative salt balance.

5. The method of claim 2, wherein the reference subjects are subjects suffering from a disease or disorder which is known to be associated with a critical extracellular volume status, such as aneurysm, multiple trauma, brain injury, and/or head injury, or wherein the reference subjects are post-operative subjects suffering from peritonitis with shock.

6. The method of claim 5, wherein
(i) a similar level, identical level or increased level of proADM or said fragment thereof, optionally MR-proADM, of the subject as compared to said reference level indicates that said subject has a positive fluid balance, a positive salt balance, a critical globular volume status and/or a critical extracellular volume status; and/or
(ii) a decreased level of proADM or said fragment thereof, optionally MR-proADM, of the subject as compared to said reference level indicates that said subject has a normal fluid balance, a normal salt balance, a normal extracellular volume status and/or a normal globular volume status.

7. The method of claim 1, wherein a level of proADM or said fragment thereof, optionally MR-proADM, of 1 nmol/L or more in the sample is indicative for a critical extracellular volume status, a critical globular volume status, a positive salt balance and/or a positive salt balance.

8. The method of claim 1, wherein said method further comprises
(i) determining the level of hemoglobin and/or the level of the total serum protein;
(ii) determining at least one parameter of the subject selected from the group consisting of body mass index, weight, age and sex; and/or
(iii) determining at least one marker and/or parameter of the subject selected from the group consisting of the level of proANP in the sample, the level of total blood volume, the level of haematocrit in the sample, the level of red blood cells volume, the level of plasmatic volume, the level of total urine volume, the level of angiotensin II in the sample, the patient group of the subject, the level of cortisol in the sample, number of endothelial stem cells in the blood, the level of catecholamines in the sample, full blood ionogram of the subject, urinary ionogram of the subject, blood osmolarity of the subject, urine osmolarity of the subject, blood sugar, the level of pro-endothelin-1 (pro-ET-1) in the sample, the level of CT-proAVP in the sample, the level of aldosterone in the sample, the level of lactate in the sample, Acute Physiology and Chronic Health Evaluation II (APACHE II) of the subject, World Federation of Neurosurgical Societies (WFNS) grading of the subject, and Glasgow Coma Scale (GCS) of the subject is determined; or
(iv) determining the body mass index of the subject, the weight of the subject, the age of the subject, the sex of the subject, the level of hemoglobin in the sample and the level of the total serum protein in the sample.

9. The method of claim 1, wherein said subject suffers from a brain injury, an aneurysm, a head injury, multiple traumatic injuries and/or wherein said subject is post-operative.

10. The method of claim 1, wherein said sample is blood, blood plasma, blood serum or urine.

11. The method of claim 1, wherein said level of proADM or said fragment thereof, optionally MR-proADM, is determined by an immunoassay, wherein said assay is performed in homogeneous phase or in heterogeneous phase.

12. Method for in vitro diagnosis, prognosis, risk assessment, risk stratification, therapy control and/or operative control of a disorder or medical condition in a subject, wherein the extracellular volume status of said subject is determined by the method of claim 1,
optionally wherein the disorder or medical condition is selected from the group consisting of edema, brain damage, post-aneurysm rupture, head injury, neurological impairment, multiple traumatic injuries, post-operative, organ failure, disregulated lymphatic flow activity, kidney dysfunction, cardiac dysfunction, disease associated with disordered fluid balance.

13. A kit for determining the extracellular volume status, the fluid balance, the salt balance and/or the globular volume status of a subject, wherein the kit comprises one or more detection reagents for determining the level of proADM or said fragment thereof, optionally MR-proADM, in a sample of said subject, optionally wherein said detection reagents comprise antibodies, wherein one of the antibodies is labelled and the other antibody is bound to a solid phase or can be bound selectively to a solid phase.

14. The kit according to claim 13, wherein a first and a second antibody are present dispersed in a liquid reaction mixture, and wherein a first labelling component that is part of a labelling system based on fluorescence or chemiluminescence extinction or amplification is bound to the first antibody, and a second labelling component of said labelling system is bound to the second antibody so that, after binding of both antibodies to proADM or said fragment thereof, optionally MR-proADM, a measurable signal which permits detection of the resulting sandwich complexes in the measuring solution is generated, optionally wherein said labelling system comprises rare earth cryptates or chelates in combination with a fluorescent or chemiluminescent dye, optionally of the cyanine type.

15. The method according to claim 1, wherein the fragment of proADM is selected from the group consisting of MR-proADM, PAMP, adrenotensin and mature adrenomedullin, optionally the fragment is MR-proADM.
